SEQUENCE LISTING (68 sequences)

SEQ ID NO: 1 - protein, 573 aa, synthetic construct
  REGION 1..573: note = Description of Artificial Sequence: Synthetic polypeptide
MDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVSSRSIFKDKQSCDIKMEGMARNDLWYLSLEEVWKCRDQLDKYQENPERHLRQVAFTQKKVLTQERVSESGKYGGNCLLPAQLVLREYFHKRDSHTKSLKHDLVLNGHQDSCASNSNECGQTFCQNIHLIQFARTHTGDKSYKCPDNDNSLTHGSSLGISKGIHREKPYECKECGKFFSWRSNLTRHQLIHTGEKPYECKECGKSFSRSSHLIGHQKTHTGEEPYECKECGKSFSWFSHLVTHQRTHTGDKLYTCNQCGKSFVHSSRLIRHQRTHTGEKPYECPECGKSFRQSTHLILHQRTHVRVRPYECNECGKSYSQRSHLVVHHRIHTGLKPFECKDCGKCFSRSSHLYSHQRTHTGEKPYECHDCGKSFSQSSALIVHQRIHTGEKPYECCQCGKAFIRKNDLIKHQRIHVGEETYKCNQCGIIFSQNSPFIVHQIAHTGEQFLTCNQCGTALVNTSNLIGYQTNHIRENAY

SEQ ID NO: 2 - protein, 81 aa, synthetic construct
  REGION 1..81: note = Description of Artificial Sequence: Synthetic polypeptide
DAKSLTAWSRTLVTFKDVFVDFTREEWKLLDTAQQIVYRNVMLENYKNLVSLGYQLTKPDVILRLEKGEEPWLVEREIHQE

SEQ ID NO: 3 - protein, 45 aa, synthetic construct
  REGION 1..45: note = Description of Artificial Sequence: Synthetic polypeptide
RTLVTFKDVFVDFTREEWKLLDTAQQIVYRNVMLENYKNLVSLGY

SEQ ID NO: 4 - protein, 1083 aa, synthetic construct
  REGION 1..1083: note = Description of Artificial Sequence: Synthetic polypeptide
SSQSHPDGLSGRDQPVELLNPARVNHMPSTVDVATALPLQVAPSAVPMDLRLDHQFSLPVAEPALREQQLQQELLALKQKQQIQRQILIAEFQRQHEQLSRQHEAQLHEHIKQQQEMLAMKHQQELLEHQRKLERHRQEQELEKQHREQKLQQLKNKEKGKESAVASTEVKMKLQEFVLNKKKALAHRNLNHCISSDPRYWYGKTQHSSLDQSSPPQSGVSTSYNHPVLGMYDAKDDFPLRKTASEPNLKLRSRLKQKVAERRSSPLLRRKDGPVVTALKKRPLDVTDSACSSAPGSGPSSPNNSSGSVSAENGIAPAVPSIPAETSLAHRLVAREGSAAPLPLYTSPSLPNITLGLPATGPSAGTAGQQDTERLTLPALQQRLSLFPGTHLTPYLSTSPLERDGGAAHSPLLQHMVLLEQPPAQAPLVTGLGALPLHAQSLVGADRVSPSIHKLRQHRPLGRTQSAPLPQNAQALQHLVIQQQHQQFLEKHKQQFQQQQLQMNKIIPKPSEPARQPESHPEETEEELREHQALLDEPYLDRLPGQKEAHAQAGVQVKQEPIESDEEEAEPPREVEPGQRQPSEQELLFRQQALLLEQQRIHQLRNYQASMEAAGIPVSFGGHRPLSRAQSSPASATFPVSVQEPPTKPRFTTGLVYDTLMLKHQCTCGSSSSHPEHAGRIQSIWSRLQETGLRGKCECIRGRKATLEELQTVHSEAHTLLYGTNPLNRQKLDSKKLLGSLASVFVRLPCGGVGVDSDTIWNEVHSAGAARLAVGCVVELVFKVATGELKNGFAVVRPPGHHAEESTPMGFCYFNSVAVAAKLLQQRLSVSKILIVDWDVHHGNGTQQAFYSDPSVLYMSLHRYDDGNFFPGSGAPDEVGTGPGVGFNVNMAFTGGLDPPMGDAEYLAAFRTVVMPIASEFAPDVVLVSSGFDAVEGHPTPLGGYNLSARCFGYLTKQLMGLAGGRIVLALEGGHDLTAICDASEACVSALLGNELDPLPEKVLQQRPNANAVRSMEKVMEIHSKYWRCLQRTTSTAGRSLIEAQTCENEEAETVTAMASLSVGVKPAEKRPDEEPMEEEPPL

SEQ ID NO: 5 - protein, 176 aa, synthetic construct
  REGION 1..176: note = Description of Artificial Sequence: Synthetic polypeptide
SRPGERPFQCRICMRNFSRRHGLDRHTRTHTGEKPFQCRICMRNFSDHSSLKRHLRTHTGSQKPFQCRICMRNFSVRHNLTRHLRTHTGEKPFQCRICMRNFSDHSNLSRHLKTHTGSQKPFQCRICMRNFSQRSSLVRHLRTHTGEKPFQCRICMRNFSESGHLKRHLRTHLRGS

SEQ ID NO: 6 - protein, 60 aa, synthetic construct
  REGION 1..60: note = Description of Artificial Sequence: Synthetic polypeptide
FNVLMVHKRSHTGERPLQCEICGFTCRQKGNLLRHIKLHTGEKPFKCHLCNYACQRRDAL

SEQ ID NO: 7 - protein, 8 aa, synthetic construct
  REGION 1..8: note = Description of Artificial Sequence: Synthetic peptide
GSGSGSGS

SEQ ID NO: 8 - protein, 4 aa, synthetic construct
  REGION 1..4: note = Description of Artificial Sequence: Synthetic peptide
KEGS

SEQ ID NO: 9 - protein, 5 aa, synthetic construct
  REGION 1..5: note = Description of Artificial Sequence: Synthetic peptide
EAAAK

SEQ ID NO: 10 - protein, 7 aa, synthetic construct
  REGION 1..7: note = Description of Artificial Sequence: Synthetic peptide
AAPAKQE

SEQ ID NO: 11 - protein, 27 aa, synthetic construct
  REGION 1..27: note = Description of Artificial Sequence: Synthetic peptide
GSGSGSGSGGAEAAAKEAAAKEAAAKA

SEQ ID NO: 12 - protein, 44 aa, synthetic construct
  REGION 1..44: note = Description of Artificial Sequence: Synthetic polypeptide
AAPAKQEAAAPAKQEAAAPAKQEAAAPAPAAKAEAPAAAPAAKA

SEQ ID NO: 13 - protein, 17 aa, synthetic construct
  REGION 1..17: note = Description of Artificial Sequence: Synthetic peptide
AEAAAKEAAAKEAAAKA

SEQ ID NO: 14 - skipped

SEQ ID NO: 15 - DNA, 21 bp, synthetic construct
  misc_feature 1..21: note = Description of Artificial Sequence: Synthetic oligonucleotide
  source 1..21: mol_type = other DNA
atttatttatttatttattta

SEQ ID NO: 16 - DNA, 19 bp, synthetic construct
  misc_feature 1..19: note = Description of Artificial Sequence: Synthetic oligonucleotide
  source 1..19: mol_type = other DNA
ctgtttaatatttaaacag

SEQ ID NO: 17 - DNA, 51 bp, synthetic construct
  misc_feature 1..51: note = Description of Artificial Sequence: Synthetic oligonucleotide
  source 1..51: mol_type = other DNA
atttatttatttatttatttaacatcggttccctgtttaatatttaaacag

SEQ ID NO: 18 - DNA, 112 bp, synthetic construct
  misc_feature 1..112: note = Description of Artificial Sequence: Synthetic polynucleotide
  source 1..112: mol_type = other DNA
atttatttatttatttatttaacatcggttccctgtttaatatttaaacagtgcggtaagcatttatttatttatttatttaacatcggttccctgtttaatatttaaacag

SEQ ID NO: 19 - DNA, 31 bp, synthetic construct
  misc_feature 1..31: note = Description of Artificial Sequence: Synthetic oligonucleotide
  source 1..31: mol_type = other DNA
agagggtatataatggaagctcgacttccag

SEQ ID NO: 20 - DNA, 52 bp, synthetic construct
  misc_feature 1..52: note = Description of Artificial Sequence: Synthetic oligonucleotide
  source 1..52: mol_type = other DNA
gggaatttccggggactttccgggaatttccggggactttccgggaatttcc

SEQ ID NO: 21 - DNA, 85 bp, synthetic construct
  misc_feature 1..85: note = Description of Artificial Sequence: Synthetic oligonucleotide
  source 1..85: mol_type = other DNA
caccagacagtgacgtcagctgccagatcccatggccgtcatactgtgacgtctttcagacaccccattgacgtcaatgggagaa

SEQ ID NO: 22 - DNA, 90 bp, synthetic construct
  misc_feature 1..90: note = Description of Artificial Sequence: Synthetic oligonucleotide
  source 1..90: mol_type = other DNA
ggaggaaaaactgtttcatacagaaggcgtggaggaaaaactgtttcatacagaaggcgtggaggaaaaactgtttcatacagaaggcgt

SEQ ID NO: 23 - DNA, 115 bp, synthetic construct
  misc_feature 1..115: note = Description of Artificial Sequence: Synthetic polynucleotide
  source 1..115: mol_type = other DNA
aggatgtccatattaggacatctaggatgtccatattaggacatctaggatgtccatattaggacatctaggatgtccatattaggacatctaggatgtccatattaggacatct

SEQ ID NO: 24 - DNA, 115 bp, synthetic construct
  misc_feature 1..115: note = Description of Artificial Sequence: Synthetic polynucleotide
  source 1..115: mol_type = other DNA
agtatgtccatattaggacatctaccatgtccatattaggacatctactatgtccatattaggacatcttgtatgtccatattaggacatctaaaatgtccatattaggacatct

SEQ ID NO: 25 - DNA, 48 bp, synthetic construct
  misc_feature 1..48: note = Description of Artificial Sequence: Synthetic oligonucleotide
  source 1..48: mol_type = other DNA
tgagtcagtgactcagtgagtcagtgactcagtgagtcagtgactcag

SEQ ID NO: 26 - DNA, 120 bp, synthetic construct
  misc_feature 1..120: note = Description of Artificial Sequence: Synthetic polynucleotide
  source 1..120: mol_type = other DNA
agatcaaagggtttaagatcaaagggcttaagatcaaagggtataagatcaaagggcctaagatcaaagggactaagatcaaagggtttaagatcaaagggcttaagatcaaagggccta

SEQ ID NO: 27 - DNA, 32 bp, synthetic construct
  misc_feature 1..32: note = Description of Artificial Sequence: Synthetic oligonucleotide
  source 1..32: mol_type = other DNA
gtctagacgtctagacgtctagacgtctagac

SEQ ID NO: 28 - DNA, 24 bp, synthetic construct
  misc_feature 1..24: note = Description of Artificial Sequence: Synthetic oligonucleotide
  source 1..24: mol_type = other DNA
cagacacagacacagacacagaca

SEQ ID NO: 29 - DNA, 65 bp, synthetic construct
  misc_feature 1..65: note = Description of Artificial Sequence: Synthetic oligonucleotide
  source 1..65: mol_type = other DNA
ggatccggtactcgagatctgcgatctaagtaagcttggcattccggtactgttggtaaagccac

SEQ ID NO: 30 - DNA, 588 bp, synthetic construct
  misc_feature 1..588: note = Description of Artificial Sequence: Synthetic polynucleotide
  source 1..588: mol_type = other DNA
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctc

SEQ ID NO: 31 - DNA, 1179 bp, synthetic construct
  misc_feature 1..1179: note = Description of Artificial Sequence: Synthetic polynucleotide
  source 1..1179: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgccgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcgaccaccgagaatcggacgggggtagtctcaagctggccggcctgctctggtgcctgtcctcgcgccgccgtgtatcgccccgccccgggcggcaaggctggcccggtcggcaccagttgcgtgagcggaaagatggccgcttcccggtcctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 32 - DNA, 544 bp, synthetic construct
  misc_feature 1..544: note = Description of Artificial Sequence: Synthetic polynucleotide
  source 1..544: mol_type = other DNA
ggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctac

SEQ ID NO: 33 - DNA, 399 bp, synthetic construct
  misc_feature 1..399: note = Description of Artificial Sequence: Synthetic polynucleotide
  source 1..399: mol_type = other DNA
tttatttagtctccagaaaaaggggggaatgaaagaccccacctgtaggtttggcaagctaggatcaaggttaggaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagccca

SEQ ID NO: 34 - DNA, 511 bp, synthetic construct
  misc_feature 1..511: note = Description of Artificial Sequence: Synthetic polynucleotide
  source 1..511: mol_type = other DNA
ggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcggggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccag

SEQ ID NO: 35 - DNA, 408 bp, synthetic construct
  misc_feature 1..408: note = Description of Artificial Sequence: Synthetic polynucleotide
  source 1..408: mol_type = other DNA
gtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccggg

SEQ ID NO: 36 - DNA, 344 bp, synthetic construct
  misc_feature 1..344: note = Description of Artificial Sequence: Synthetic polynucleotide
  source 1..344: mol_type = other DNA
ctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagct

SEQ ID NO: 37 - DNA, 1229 bp, synthetic construct
  misc_feature 1..1229: note = Description of Artificial Sequence: Synthetic polynucleotide
  source 1..1229: mol_type = other DNA
gcgccgggttttggcgcctcccgcgggcgcccccctcctcacggcgagcgctgccacgtcagacgaagggcgcaggagcgttcctgatccttccgcccggacgctcaggacagcggcccgctgctcataagactcggccttagaaccccagtatcagcagaaggacattttaggacgggacttgggtgactctagggcactggttttctttccagagagcggaacaggcgaggaaaagtagtcccttctcggcgattctgcggagggatctccgtggggcggtgaacgccgatgattatataaggacgcgccgggtgtggcacagctagttccgtcgcagccgggatttgggtcgcggttcttgtttgtggatcgctgtgatcgtcacttggtgagttgcgggctgctgggctggccggggctttcgtggccgccgggccgctcggtgggacggaagcgtgtggagagaccgccaagggctgtagtctgggtccgcgagcaaggttgccctgaactgggggttggggggagcgcacaaaatggcggctgttcccgagtcttgaatggaagacgcttgtaaggcgggctgtgaggtcgttgaaacaaggtggggggcatggtgggcggcaagaacccaaggtcttgaggccttcgctaatgcgggaaagctcttattcgggtgagatgggctggggcaccatctggggaccctgacgtgaagtttgtcactgactggagaactcgggtttgtcgtctggttgcgggggcggcagttatgcggtgccgttgggcagtgcacccgtacctttgggagcgcgcgcctcgtcgtgtcgtgacgtcacccgttctgttggcttataatgcagggtggggccacctgccggtaggtgtgcggtaggcttttctccgtcgcaggacgcagggttcgggcctagggtaggctctcctgaatcgacaggcgccggacctctggtgaggggagggataagtgaggcgtcagtttctttggtcggttttatgtacctatcttcttaagtagctgaagctccggttttgaactatgcgctcggggttggcgagtgtgttttgtgaagttttttaggcaccttttgaaatgtaatcatttgggtcaatatgtaattttcagtgttagactagtaaagcttctgcaggtcgactctagaaaattgtccgctaaattctggccgtttttggcttttttgttagac

SEQ ID NO: 38 - DNA, 1179 bp, synthetic construct
  misc_feature 1..1179: note = Description of Artificial Sequence: Synthetic polynucleotide
  source 1..1179: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 39 - DNA, 1718 bp, synthetic construct
  misc_feature 1..1718: note = Description of Artificial Sequence: Synthetic polynucleotide
  source 1..1718: mol_type = other DNA
actagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcggggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattc

SEQ ID NO: 40 - DNA, 212 bp, synthetic construct
  misc_feature 1..212: note = Description of Artificial Sequence: Synthetic polynucleotide
  source 1..212: mol_type = other DNA
gggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 41 - DNA, 2379 bp, synthetic construct
  misc_feature 1..2379: note = Description of Artificial Sequence: Synthetic polynucleotide
  source 1..2379: mol_type = other DNA
ccactagttccatgtccttatatggactcatctttgcctattgcgacacacactcaatgaacacctactacgcgctgcaaagagccccgcaggcctgaggtgcccccacctcaccactcttcctatttttgtgtaaaaatccagcttcttgtcaccacctccaaggagggggaggaggaggaaggcaggttcctctaggctgagccgaatgcccctctgtggtcccacgccactgatcgctgcatgcccaccacctgggtacacacagtctgtgattcccggagcagaacggaccctgcccacccggtcttgtgtgctactcagtggacagacccaaggcaagaaagggtgacaaggacagggtcttcccaggctggctttgagttcctagcaccgccccgcccccaatcctctgtggcacatggagtcttggtccccagagtcccccagcggcctccagatggtctgggagggcagttcagctgtggctgcgcatagcagacatacaacggacggtgggcccagacccaggctgtgtagacccagcccccccgccccgcagtgcctaggtcacccactaacgccccaggcctggtcttggctgggcgtgactgttaccctcaaaagcaggcagctccagggtaaaaggtgccctgccctgtagagcccaccttccttcccagggctgcggctgggtaggtttgtagccttcatcacgggccacctccagccactggaccgctggcccctgccctgtcctggggagtgtggtcctgcgacttctaagtggccgcaagccacctgactcccccaacaccacactctacctctcaagcccaggtctctccctagtgacccacccagcacatttagctagctgagccccacagccagaggtcctcaggccctgctttcagggcagttgctctgaagtcggcaagggggagtgactgcctggccactccatgccctccaagagctccttctgcaggagcgtacagaacccagggccctggcacccgtgcagaccctggcccaccccacctgggcgctcagtgcccaagagatgtccacacctaggatgtcccgcggtgggtggggggcccgagagacgggcaggccgggggcaggcctggccatgcggggccgaaccgggcactgcccagcgtggggcgcgggggccacggcgcgcgcccccagcccccgggcccagcaccccaaggcggccaacgccaaaactctccctcctcctcttcctcaatctcgctctcgctctttttttttttcgcaaaaggaggggagagggggtaaaaaaatgctgcactgtgcggcgaagccggtgagtgagcggcgcggggccaatcagcgtgcgccgttccgaaagttgccttttatggctcgagcggccgcggcggcgccctataaaacccagcggcgcgacgcgccaccaccgccgagaccgcgtccgccccgcgagcacagagcctcgcctttgccgatccgccgcccgtccacacccgccgccaggtaagcccggccagccgaccggggcaggcggctcacggcccggccgcaggcggccgcggccccttcgcccgtgcagagccgccgtctgggccgcagcggggggcgcatggggggggaaccggaccgccgtggggggcgcgggagaagcccctgggcctccggagatgggggacaccccacgccagttcggaggcgcgaggccgcgctcgggaggcgcgctccgggggtgccgctctcggggcgggggcaaccggcggggtctttgtctgagccgggctcttgccaatggggatcgcagggtgggcgcggcggagcccccgccaggcccggtgggggctggggcgccattgcgcgtgcgcgctggtcctttgggcgctaactgcgtgcgcgctgggaattggcgctaattgcgcgtgcgcgctgggactcaaggcgctaactgcgcgtgcgttctggggcccggggtgccgcggcctgggctggggcgaaggcgggctcggccggaaggggtggggtcgccgcggctcccgggcgcttgcgcgcacttcctgcccgagccgctggccgcccgagggtgtggccgctgcgtgcgcgcgcgccgacccggcgctgtttgaaccgggcggaggcggggctggcgcccggttgggagggggttggggcctggcttcctgccgcgcgccgcggggacgcctccgaccagtgtttgccttttatggtaataacgcggccggcccggcttcctttgtccccaatctgggcgcgcgccggcgccccctggcggcctaaggactcggcgcgccggaagtggccagggcgggggcgacctcggctcacagcgcgcccggctat

SEQ ID NO: 42 - DNA, 529 bp, synthetic construct
  misc_feature 1..529: note = Description of Artificial Sequence: Synthetic polynucleotide
  source 1..529: mol_type = other DNA
gttgatttccttcatccctggcacacgtccaggcagtgtcgaatccatctctgctacaggggaaaacaaataacatttgagtccagtggagaccgggagcagaagtaaagggaagtgataacccccagagcccggaagcctctggaggctgagacctcgccccccttgcgtgatagggcctacggagccacatgaccaaggcactgtcgcctccgcacgtgtgagagtgcagggccccaagatggctgccaggcctcgaggcctgactcttctatgtcacttccgtaccggcgagaaaggcgggccctccagccaatgaggctgcggggcgggccttcaccttgataggcactcgagttatccaatggtgcctgcgggccggagcgactaggaactaacgtcatgccgagttgctgagcgccggcaggcggggccggggcggccaaaccaatgcgatggccggggcggagtcgggcgctctataagttgtcgataggcgggcactccgccctagtttctaaggaccatg

SEQ ID NO: 43 - DNA, 794 bp, synthetic construct
  misc_feature 1..794: note = Description of Artificial Sequence: Synthetic polynucleotide
  source 1..794: mol_type = other DNA
agttccccaactttcccgcctctcagcctttgaaagaaagaaaggggagggggcaggccgcgtgcagtcgcgagcggtgctgggctccggctccaattccccatctcagtcgctcccaaagtccttctgtttcatccaagcgtgtaagggtccccgtccttgactccctagtgtcctgctgcccacagtccagtcctgggaaccagcaccgatcacctcccatcgggccaatctcagtcccttcccccctacgtcggggcccacacgctcggtgcgtgcccagttgaaccaggcggctgcggaaaaaaaaaagcggggagaaagtagggcccggctactagcggttttacgggcgcacgtagctcaggcctcaagaccttgggctgggactggctgagcctggcgggaggcggggtccgagtcaccgcctgccgccgcgcccccggtttctataaattgagcccgcagcctcccgcttcgctctctgctcctcctgttcgacagtcagccgcatcttcttttgcgtcgccaggtgaagacgggcggagagaaacccgggaggctagggacggcctgaaggcggcaggggcgggcgcaggccggatgtgttcgcgccgctgcggggtgggcccgggcggcctccgcattgcaggggcgggcggaggacgtgatgcggcgcgggctgggcatggaggcctggtgggggaggggaggggaggcgtgggtgtcggccggggccactaggcgctcactgttctctccctccgcgcagccgagccacatcgctgagacac

SEQ ID NO: 44 - DNA, 532 bp, synthetic construct
  misc_feature 1..532: note = Description of Artificial Sequence: Synthetic polynucleotide
  source 1..532: mol_type = other DNA
agtgcggttaccagcggaaatgcctcggggtcagaagtcgcaggagagatagacagctgctgaaccaatgggaccagcggatggggcggatgttatctaccattggtgaacgttagaaacgaatagcagccaatgaatcagctgggggggcggagcagtgacgtttattgcggagggggccgcttcgaatcggcggcggccagcttggtggcctgggccaatgaacggcctccaacgagcagggccttcaccaatcggcggcctccacgacggggctgggggagggtatataagccgagtaggcgacggtgaggtcgacgccggccaagacagcacagacagattgacctattggggtgtttcgcgagtgtgagagggaagcgccgcggcctgtatttctagacctgcccttcgcctggttcgtggcgccttgtgaccccgggcccctgccgcctgcaagtcggaaattgcgctgtgctcctgtgctacggcctgtggctggactgcctgctgctgcccaactggctggcac

SEQ ID NO: 45 - DNA, 701 bp, synthetic construct
  misc_feature 1..701: note = Description of Artificial Sequence: Synthetic polynucleotide
  source 1..701: mol_type = other DNA
tagtttcatcaccaccgccacccccccgcccccccgccatctgaaagggttctaggggatttgcaacctctctcgtgtgtttcttctttccgagaagcgccgccacacgagaaagctggccgcgaaagtcgtgctggaatcacttccaacgaaaccccaggcatagatgggaaagggtgaagaacacgttgccatggctaccgtttccccggtcacggaataaacgctctctaggatccggaagtagttccgccgcgacctctctaaaaggatggatgtgttctctgcttacattcattggacgttttcccttagaggccaaggccgcccaggcaaaggggcggtcccacgcgtgaggggcccgcggagccatttgattggagaaaagctgcaaaccctgaccaatcggaaggagccacgcttcgggcatcggtcaccgcacctggacagctccgattggtggacttccgccccccctcacgaatcctcattgggtgccgtgggtgcgtggtgcggcgcgattggtgggttcatgtttcccgtcccccgcccgcgagaagtgggggtgaaaagcggcccgacctgcttggggtgtagtgggcggaccgcgcggctggaggtgtgaggatccgaacccaggggtggggggtggaggcggctcctgcgatcgaaggggacttgagactcaccggccgcacgtc

SEQ ID NO: 46 - DNA, 465 bp, synthetic construct
  misc_feature 1..465: note = Description of Artificial Sequence: Synthetic polynucleotide
  source 1..465: mol_type = other DNA
gggccgcccactcccccttcctctcagggtccctgtcccctccagtgaatcccagaagactctggagagttctgagcagggggcggcactctggcctctgattggtccaaggaaggctggggggcaggacgggaggcgaaaaccctggaatattcccgacctggcagcctcatcgagctcggtgattggctcagaagggaaaaggcgggtctccgtgacgacttataaaagcccaggggcaagcggtccggataacggctagcctgaggagctgctgcgacagtccactacctttttcgagagtgactcccgttgtcccaaggcttcccagagcgaacctgtgcggctgcaggcaccggcgcgtcgagtttccggcgtccggaaggaccgagctcttctcgcggatccagtgttccgtttccagcccccaatctcagagcggagccgacagagagcagggaaccc

SEQ ID NO: 47 - DNA, 538 bp, synthetic construct
  misc_feature 1..538: note = Description of Artificial Sequence: Synthetic polynucleotide
  source 1..538: mol_type = other DNA
gccccacccccgtccgcgttacaaccgggaggcccgctgggtcctgcaccgtcaccctcctccctgtgaccgcccacctgatacccaaacaactttctcgcccctccagtccccagctcgccgagcgcttgcggggagccacccagcctcagtttccccagccccgggcggggcgaggggcgatgacgtcatgccggcgcgcggcattgtggggcggggcgaggcggggcgccggggggagcaacactgagacgccattttcggcggcgggagcggcgcaggcggccgagcgggactggctgggtcggctgggctgctggtgcgaggagccgcggggctgtgctcggcggccaaggggacagcgcgtgggtggccgaggatgctgcggggcggtagctccggcgcccctcgctggtgactgctgcgccgtgcctcacacagccgaggcgggctcggcgcacagtcgctgctccgcgctcgcgcccggcggcgctccaggtgctgacagcgcgagagagcgcggcctcaggagcaacac

SEQ ID NO: 48 - DNA, 1091 bp, synthetic construct
  misc_feature 1..1091: note = Description of Artificial Sequence: Synthetic polynucleotide
  source 1..1091: mol_type = other DNA
ttccagagctttcgaggaaggtttcttcaactcaaattcatccgcctgataattttcttatattttcctaaagaaggaagagaagcgcatagaggagaagggaaataattttttaggagcctttcttacggctatgaggaatttggggctcagttgaaaagcctaaactgcctctcgggaggttgggcgcggcgaactactttcagcggcgcacggagacggcgtctacgtgaggggtgataagtgacgcaacactcgttgcataaatttgcgctccgccagcccggagcatttaggggcggttggctttgttgggtgagcttgtttgtgtccctgtgggtggacgtggttggtgattggcaggatcctggtatccgctaacaggtactggcccacagccgtaaagacctgcgggggcgtgagaggggggaatgggtgaggtcaagctggaggcttcttggggttgggtgggccgctgaggggaggggagggcgaggtgacgcgacacccggcctttctgggagagtgggccttgttgacctaaggggggcgagggcagttggcacgcgcacgcgccgacagaaactaacagacattaaccaacagcgattccgtcgcgtttacttgggaggaaggcggaaaagaggtagtttgtgtggcttctggaaaccctaaatttggaatcccagtatgagaatggtgtcccttcttgtgtttcaatgggatttttacttcgcgagtcttgtgggtttggttttgttttcagtttgcctaacaccgtgcttaggtttgaggcagattggagttcggtcgggggagtttgaatatccggaacagttagtggggaaagctgtggacgcttggtaagagagcgctctggattttccgctgttgacgttgaaaccttgaatgacgaatttcgtattaagtgacttagccttgtaaaattgaggggaggcttgcggaatattaacgtatttaaggcattttgaaggaatagttgctaattttgaagaatattaggtgtaaaagcaagaaatacaatgatcctgaggtgacacgcttatgttttacttttaaactaggtcacc

SEQ ID NO: 49 - protein, 415 aa, synthetic construct
  REGION 1..415: note = Description of Artificial Sequence: Synthetic polypeptide
DEADRRLLRRCRLRLVEELQVDQLWDVLLSRELFRPHMIEDIQRAGSGSRRDQARQLIIDLETRGSQALPLFISCLEDTGQDMLASFLRTNRQAAKLSKPTLENLTPVVLRPEIRKPEVLRPETPRPVDIGSGGFGDVGALESLRGNADLAYILSMEPCGHCLIINNVNFCRESGLRTRTGSNIDCEKLRRRFSSLHFMVEVKGDLTAKKMVLALLELARQDHGALDCCVVVILSHGCQASHLQFPGAVYGTDGCPVSVEKIVNIFNGTSCPSLGGKPKLFFIQACGGEQKDHGFEVASTSPEDESPGSNPEPDATPFQEGLRTFDQLDAISSLPTPSDIFVSYSTFPGFVSWRDPKSGSWYVETLDDIFEQWAHSEDLQSLLLRVANAVSVKGIYKQMPGCFNFLRKKLFFKTS

SEQ ID NO: 50 - protein, 217 aa, synthetic construct
  REGION 1..217: note = Description of Artificial Sequence: Synthetic polypeptide
DPDDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSLCEGTLLLWCDIIGQTTYRDLKL

SEQ ID NO: 51 - protein, 246 aa, synthetic construct
  REGION 1..246: note = Description of Artificial Sequence: Synthetic polypeptide
QPILLLLAFLLLPRADAGEIIGGHEAKPHSRPYMAYLMIWDQKSLKRCGGFLIQDDFVLTAAHCWGSSINVTLGAHNIKEQEPTQQFIPVKRPIPHPAYNPKNFSNDIMLLQLERKAKRTRAVQPLRLPSNKAQVKPGQTCSVAGWGQTAPLGKHSHTLQEVKMTVQEDRKCESDLRHYYDSTIELCVGDPEIKKTSFKGDSGGPLVCNKVAQGIVSYGRNNGMPPRACTKVSSFVHWIKKTMKRH

SEQ ID NO: 52 - protein, 191 aa, synthetic construct
  REGION 1..191: note = Description of Artificial Sequence: Synthetic polypeptide
DGSGEQPRGGGPTSSEQIMKTGALLLQGFIQDRAGRMGGEAPELALDPVPQDASTKKLSECLKRIGDELDSNMELQRMIAAVDTDSPREVFFRVAADMFSDGNFNWGRVVALFYFASKLVLKALCTKVPELIRTIMGWTLDFLRERLLGWIQDQGGWDGLLSYFGTPTWQTVTIFVAGVLTASLTIWKKMG

SEQ ID NO: 53 - protein, 503 aa, synthetic construct
  REGION 1..503: note = Description of Artificial Sequence: Synthetic polypeptide
SRGSEFMTFNSFEGSKTCVPADINKEEEFVEEFNRLKTFANFPSGSPVSASTLARAGFLYTGEGDTVRCFSCHAAVDRWQYGDSAVGRHRKVSPNCRFINGFYLENSATQSTNSGIQNGQYKVENYLGSRDHFALDRPSETHADYLLRTGQVVDISDTIYPRNPAMYSEEARLKSFQNWPDYAHLTPRELASAGLYYTGIGDQVQCFCCGGKLKNWEPCDRAWSEHRRHFPNCFFVLGRNLNIRSESDAVSSDRNFPNSTNLPRNPSMADYEARIFTFGTWIYSVNKEQLARAGFYALGEGDKVKCFHCGGGLTDWKPSEDPWEQHAKWYPGCKYLLEQKGQEYINNIHLTHSLEECLVRTTEKTPSLTRRIDDTIFQNPMVQEAIRMGFSFKDIKKIMEEKIQISGSNYKSLEVLVADLVNAQKDSMQDESSQTSLQKEISTEEQLRRLQEEKLCKICMDRNIAIVFVPCGHLVTCKQCAEAVDKCPMCYTVITFKQKIFMS

SEQ ID NO: 54 - DNA, 18 bp, synthetic construct
  misc_feature 1..18: note = Description of Artificial Sequence: Synthetic oligonucleotide
  source 1..18: mol_type = other DNA
ggcgtagccgatgtcgcg

SEQ ID NO: 55 - DNA, 172 bp, synthetic construct
  misc_feature 1..172: note = Description of Artificial Sequence: Synthetic polynucleotide
  source 1..172: mol_type = other DNA
cgggtttcgtaacaatcgcatgaggattcgcaacgccttcggcgtagccgatgtcgcgctcccgtctcagtaaaggtcggcgtagccgatgtcgcgcaatcggactgccttcgtacggcgtagccgatgtcgcgcgtatcagtcgcctcggaacggcgtagccgatgtcgcg

SEQ ID NO: 56 - DNA, 25 bp, synthetic construct
  misc_feature 1..25: note = Description of Artificial Sequence: Synthetic oligonucleotide
  source 1..25: mol_type = other DNA
tctagagggtatataatgggggcca

SEQ ID NO: 57 - DNA, 243 bp, synthetic construct
  misc_feature 1..243: note = Description of Artificial Sequence: Synthetic polynucleotide
  source 1..243: mol_type = other DNA
cgggtttcgtaacaatcgcatgaggattcgcaacgccttcggcgtagccgatgtcgcgctcccgtctcagtaaaggtcggcgtagccgatgtcgcgcaatcggactgccttcgtacggcgtagccgatgtcgcgcgtatcagtcgcctcggaacggcgtagccgatgtcgcgcattcgtaagaggctcactctcccttacacggagtggataactagttctagagggtatataatgggggcca

SEQ ID NO: 58 - DNA, 672 bp, synthetic construct
  misc_feature 1..672: note = Description of Artificial Sequence: Synthetic polynucleotide
  source 1..672: mol_type = other DNA
tctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaagctgacgtcctttccatggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctgtttcgcctcggcgtccggtccgtgttgcttggtcgtcacctgtgcagaattgcgaaccatggattcca

SEQ ID NO: 59 - DNA, 672 bp, synthetic construct
  misc_feature 1..672: note = Description of Artificial Sequence: Synthetic polynucleotide
  source 1..672: mol_type = other DNA
tctgttcctgttaatcaacctctggattacaaaatttgtgaaagattgactgatattcttaactatgttgctccttttacgctgtgtggatatgctgctttaatgcctctgtatcatgctattgcttcccgtacggctttcgttttctcctccttgtataaatcctggttgctgtctctttatgaggagttgtggcccgttgtccgtcaacgtggcgtggtgtgctctgtgtttgctgacgcaacccccactggctggggcattgccaccacctgtcaactcctttctgggactttcgctttccccctcccgatcgccacggcagaactcatcgccgcctgccttgcccgctgctggacaggggctaggttgctgggcactgataattccgtggtgttgtcggggaaatcatcgtcctttccttggctgctcgcctgtgttgccaactggatcctgcgcgggacgtccttctgctacgtcccttcggctctcaatccagcggacctcccttcccgaggccttctgccggttctgcggcctctcccgcgtcttcgctttcggcctccgacgagtcggatctccctttgggccgcctccccgcctgtttcgcctcggcgtccggtccgtgttgcttggtcgtcacctgtgcagaattgcgaaccatggattcca

SEQ ID NO: 60 - protein, 8 aa, synthetic construct
  REGION 1..8: note = Description of Artificial Sequence: Synthetic peptide
GGGGSGGT

SEQ ID NO: 61 - protein, 340 aa, synthetic construct
  REGION 1..340: note = Description of Artificial Sequence: Synthetic polypeptide
FNVLMVHKRSHTGERPLQCEICGFTCRQKGNLLRHIKLHTGEKPFKCHLCNYACQRRDALAEAAAKEAAAKEAAAKADYKDHDGDYKDHDIDYKDDDDKMAPKKKRKVTCRSRPGERPFQCRICMRNFSRRHGLDRHTRTHTGEKPFQCRICMRNFSDHSSLKRHLRTHTGSQKPFQCRICMRNFSVRHNLTRHLRTHTGEKPFQCRICMRNFSDHSNLSRHLKTHTGSQKPFQCRICMRNFSQRSSLVRHLRTHTGEKPFQCRICMRNFSESGHLKRHLRTHLRGSGGGGSGGTRTLVTFKDVFVDFTREEWKLLDTAQQIVYRNVMLENYKNLVSLGY

SEQ ID NO: 62 - DNA, 1181 bp, synthetic construct
  misc_feature 1..1181: note = Description of Artificial Sequence: Synthetic polynucleotide
  source 1..1181: mol_type = other DNA
ttcaacgtgctgatggtgcacaagcggagccacaccggcgaaagacctctgcagtgtgaaatctgcggcttcacctgtcggcagaagggcaacctgctgcggcacatcaaactgcacacaggcgagaagcccttcaagtgccacctgtgcaattacgcctgccagagaagagatgccctggccgaagccgccgctaaagaagctgctgccaaagaggccgctgccaaggccgattacaaggaccacgatggcgactataaggatcacgacatcgactacaaggacgatgacgacaagatggcccctaagaaaaagcggaaagtgacctgcagaagcagacccggcgagaggcctttccagtgcagaatctgcatgcggaacttcagcagacggcacggcctggacagacacaccagaacacataccggggagaagccatttcagtgccggatctgtatgcgcaatttctccgaccacagcagcctgaagcggcacctgagaacacacacaggcagccagaaaccattccaatgtcggatctgcatgagaaatttcagcgtgcggcacaacctgaccagacacctgaggacccatactggcgaaaaacccttccagtgtcgcatatgtatgaggaactttagcgaccactccaacctgagccgccacctgaaaacccacaccggatctcagaaaccttttcagtgtaggatatgcatgcgcaactttagccagcggagcagccttgtgcgccatctgagaactcacactggggagaaaccctttcaatgccgaatatgcatgcgaaatttttccgagagcggccacctcaagcggcatctgcgtacacaccttagaggatctggcggaggtggcagcggaggcacaagaaccctggtcaccttcaaggacgtgttcgtggacttcacccgggaagagtggaagctgctggatacagcccagcagatcgtgtaccggaacgtgatgctggaaaactacaagaatctggtgtccctgggctactgactgcaggcatgcgtgactgactgaggccgcgactctagtttaaacatttatttatttatttatttaacatcggttccctgtttaatatttaaacagtgcggtaagcatttatttatttatttatttaacatcggttccctgtttaatatttaaacaga

SEQ ID NO: 63 - protein, 1378 aa, synthetic construct
  REGION 1..1378: note = Description of Artificial Sequence: Synthetic polypeptide
FNVLMVHKRSHTGERPLQCEICGFTCRQKGNLLRHIKLHTGEKPFKCHLCNYACQRRDALAEAAAKEAAAKEAAAKASSQSHPDGLSGRDQPVELLNPARVNHMPSTVDVATALPLQVAPSAVPMDLRLDHQFSLPVAEPALREQQLQQELLALKQKQQIQRQILIAEFQRQHEQLSRQHEAQLHEHIKQQQEMLAMKHQQELLEHQRKLERHRQEQELEKQHREQKLQQLKNKEKGKESAVASTEVKMKLQEFVLNKKKALAHRNLNHCISSDPRYWYGKTQHSSLDQSSPPQSGVSTSYNHPVLGMYDAKDDFPLRKTASEPNLKLRSRLKQKVAERRSSPLLRRKDGPVVTALKKRPLDVTDSACSSAPGSGPSSPNNSSGSVSAENGIAPAVPSIPAETSLAHRLVAREGSAAPLPLYTSPSLPNITLGLPATGPSAGTAGQQDTERLTLPALQQRLSLFPGTHLTPYLSTSPLERDGGAAHSPLLQHMVLLEQPPAQAPLVTGLGALPLHAQSLVGADRVSPSIHKLRQHRPLGRTQSAPLPQNAQALQHLVIQQQHQQFLEKHKQQFQQQQLQMNKIIPKPSEPARQPESHPEETEEELREHQALLDEPYLDRLPGQKEAHAQAGVQVKQEPIESDEEEAEPPREVEPGQRQPSEQELLFRQQALLLEQQRIHQLRNYQASMEAAGIPVSFGGHRPLSRAQSSPASATFPVSVQEPPTKPRFTTGLVYDTLMLKHQCTCGSSSSHPEHAGRIQSIWSRLQETGLRGKCECIRGRKATLEELQTVHSEAHTLLYGTNPLNRQKLDSKKLLGSLASVFVRLPCGGVGVDSDTIWNEVHSAGAARLAVGCVVELVFKVATGELKNGFAVVRPPGHHAEESTPMGFCYFNSVAVAAKLLQQRLSVSKILIVDWDVHHGNGTQQAFYSDPSVLYMSLHRYDDGNFFPGSGAPDEVGTGPGVGFNVNMAFTGGLDPPMGDAEYLAAFRTVVMPIASEFAPDVVLVSSGFDAVEGHPTPLGGYNLSARCFGYLTKQLMGLAGGRIVLALEGGHDLTAICDASEACVSALLGNELDPLPEKVLQQRPNANAVRSMEKVMEIHSKYWRCLQRTTSTAGRSLIEAQTCENEEAETVTAMASLSVGVKPAEKRPDEEPMEEEPPLGGGGSGGTDYKDHDGDYKDHDIDYKDDDDKMAPKKKRKVTCRSRPGERPFQCRICMRNFSRRHGLDRHTRTHTGEKPFQCRICMRNFSDHSSLKRHLRTHTGSQKPFQCRICMRNFSVRHNLTRHLRTHTGEKPFQCRICMRNFSDHSNLSRHLKTHTGSQKPFQCRICMRNFSQRSSLVRHLRTHTGEKPFQCRICMRNFSESGHLKRHLRTHLRGS

SEQ ID NO: 64 - DNA, 4137 bp, synthetic construct
  misc_feature 1..4137: note = Description of Artificial Sequence: Synthetic polynucleotide
  source 1..4137: mol_type = other DNA
ttcaacgtgctgatggtgcacaagcggagccacaccggcgaaagacctctgcagtgtgaaatctgcggcttcacctgtcggcagaagggcaacctgctgcggcacatcaaactgcacacaggcgagaagcccttcaagtgccacctgtgcaattacgcctgccagagaagagatgccctggccgaagccgccgctaaagaagctgctgccaaagaggccgctgccaaggccagctcccaaagccatccagatggactttctggccgagaccagccagtggagctgctgaatcctgcccgcgtgaaccacatgcccagcacggtggatgtggccacggcgctgcctctgcaagtggccccctcggcagtgcccatggacctgcgcctggaccaccagttctcactgcctgtggcagagccggccctgcgggagcagcagctgcagcaggagctcctggcgctcaagcagaagcagcagatccagaggcagatcctcatcgctgagttccagaggcagcacgagcagctctcccggcagcacgaggcgcagctccacgagcacatcaagcaacaacaggagatgctggccatgaagcaccagcaggagctgctggaacaccagcggaagctggagaggcaccgccaggagcaggagctggagaagcagcaccgggagcagaagctgcagcagctcaagaacaaggagaagggcaaagagagtgccgtggccagcacagaagtgaagatgaagttacaagaatttgtcctcaataaaaagaaggcgctggcccaccggaatctgaaccactgcatttccagcgaccctcgctactggtacgggaaaacgcagcacagttcccttgaccagagttctccaccccagagcggagtgtcgacctcctataaccacccggtcctgggaatgtacgacgccaaagatgacttccctcttaggaaaacagcttctgaaccgaatctgaaattacggtccaggctaaagcagaaagtggccgaaagacggagcagccccctgttacgcaggaaagacgggccagtggtcactgctctaaaaaagcgtccgttggatgtcacagactccgcgtgcagcagcgccccaggctccggacccagctcacccaacaacagctccgggagcgtcagcgcggagaacggtatcgcgcccgccgtccccagcatcccggcggagacgagtttggcgcacagacttgtggcacgagaaggctcggccgctccacttcccctctacacatcgccatccttgcccaacatcacgctgggcctgcctgccaccggcccctctgcgggcacggcgggccagcaggacaccgagagactcacccttcccgccctccagcagaggctctcccttttccccggcacccacctcactccctacctgagcacctcgcccttggagcgggacggaggggcagcgcacagccctcttctgcagcacatggtcttactggagcagccaccggcacaagcacccctcgtcacaggcctgggagcactgcccctccacgcacagtccttggttggtgcagaccgggtgtccccctccatccacaagctgcggcagcaccgcccactggggcggacccagtcggccccgctgccccagaacgcccaggctctgcagcacctggtcatccagcagcagcatcagcagtttctggagaaacacaagcagcagttccagcagcagcaactgcagatgaacaagatcatccccaagccaagcgagccagcccggcagccggagagccacccggaggagacggaggaggagctccgtgagcaccaggctctgctggacgagccctacctggaccggctgccggggcagaaggaggcgcacgcacaggccggcgtgcaggtgaagcaggagcccattgagagcgatgaggaagaggcagagcccccacgggaggtggagccgggccagcgccagcccagtgagcaggagctgctcttcagacagcaagccctcctgctggagcagcagcggatccaccagctgaggaactaccaggcgtccatggaggccgccggcatccccgtgtccttcggcggccacaggcctctgtcccgggcgcagtcctcacccgcgtctgccaccttccccgtgtctgtgcaggagccccccaccaagccgaggttcacgacaggcctcgtgtatgacacgctgatgctgaagcaccagtgcacctgcgggagtagcagcagccaccccgagcacgccgggaggatccagagcatctggtcccgcctgcaggagacgggcctccggggcaaatgcgagtgcatccgcggacgcaaggccaccctggaggagctacagacggtgcactcggaagcccacaccctcctgtatggcacgaaccccctcaaccggcagaaactggacagtaagaaacttctaggctcgctcgcctccgtgttcgtccggctcccttgcggtggtgttggggtggacagtgacaccatatggaacgaggtgcactcggcgggggcagcccgcctggctgtgggctgcgtggtagagctggtcttcaaggtggccacaggggagctgaagaatggctttgctgtggtccgcccccctggacaccatgcggaggagagcacgcccatgggcttttgctacttcaactccgtggccgtggcagccaagcttctgcagcagaggttgagcgtgagcaagatcctcatcgtggactgggacgtgcaccatggaaacgggacccagcaggctttctacagcgaccctagcgtcctgtacatgtccctccaccgctacgacgatgggaacttcttcccaggcagcggggctcctgatgaggtgggcacagggcccggcgtgggtttcaacgtcaacatggctttcaccggcggcctggacccccccatgggagacgctgagtacttggcggccttcagaacggtggtcatgccgatcgccagcgagtttgccccggatgtggtgctggtgtcatcaggcttcgatgccgtggagggccaccccacccctcttgggggctacaacctctccgccagatgcttcgggtacctgacgaagcagctgatgggcctggctggcggccggattgtcctggccctcgagggaggccacgacctgaccgccatttgcgacgcctcggaagcatgtgtttctgccttgctgggaaacgagcttgatcctctcccagaaaaggttttacagcaaagacccaatgcaaacgctgtccgttccatggagaaagtcatggagatccacagcaagtactggcgctgcctgcagcgcacaacctccacagcggggcgttctctgatcgaggctcagacttgcgagaacgaagaagccgagacggtcaccgccatggcctcgctgtccgtgggcgtgaagcccgccgaaaagagaccagatgaggagcccatggaagaggagccgcccctgggcggcggaggatctggcggcacagattacaaagaccacgacggcgactacaaggatcacgacatcgattacaaggacgacgatgacaagatggcccctaagaaaaagcggaaagtgacctgcagaagcagacccggcgaaagacccttccagtgccggatctgcatgcggaacttcagcagaaggcacggcctggacagacacaccagaacacacacaggcgagaagcctttccagtgtagaatctgtatgcgcaatttcagcgaccacagcagcctgaagcggcacctgagaacacataccggcagccagaaaccatttcaatgccgcatctgtatgagaaacttctccgtgcggcacaacctgaccagacacctgaggacccacaccggggagaaacccttccaatgcagaatatgcatgaggaatttctccgaccactccaacctgagccgccacctgaaaacccatacaggctctcaaaagccctttcaatgtcggatatgtatgcggaatttttcccagcggagcagcctcgtgcgccatctgagaactcacactggggaaaagccatttcagtgccgtatatgcatgcgcaatttctctgagagcggccacctgaagagacatctgcggacacacctgagaggctcttga

SEQ ID NO: 65 - protein, 581 aa, synthetic construct
  REGION 1..581: note = Description of Artificial Sequence: Synthetic polypeptide
FNVLMVHKRSHTGERPLQCEICGFTCRQKGNLLRHIKLHTGEKPFKCHLCNYACQRRDALAEAAAKEAAAKEAAAKADYKDHDGDYKDHDIDYKDDDDKMAPKKKRKVTCRSRPGERPFQCRICMRNFSRRHGLDRHTRTHTGEKPFQCRICMRNFSDHSSLKRHLRTHTGSQKPFQCRICMRNFSVRHNLTRHLRTHTGEKPFQCRICMRNFSDHSNLSRHLKTHTGSQKPFQCRICMRNFSQRSSLVRHLRTHTGEKPFQCRICMRNFSESGHLKRHLRTHLRGSGGGGSGGTRTLVTFKDVFVDFTREEWKLLDTAQQIVYRNVMLENYKNLVSLGYGGGGSGGTMSELIKENMHMKLYMEGTVDNHHFKCTSEGEGKPYEGTQTMRIKVVEGGPLPFAFDILATSFLYGSKTFINHTQGIPDFFKQSFPEGFTWERVTTYEDGGVLTATQDTSLQDGCLIYNVKIRGVNFTSNGPVMQKKTLGWEAFTETLYPADGGLEGRNDMALKLVGGSHLIANIKTTYRSKKPAKNLKMPGVYYVDYRLERIKEANNETYVEQHEVAVARYCDLPSKLGHKLN

SEQ ID NO: 66 - DNA, 1904 bp, synthetic construct
  misc_feature 1..1904: note = Description of Artificial Sequence: Synthetic polynucleotide
  source 1..1904: mol_type = other DNA
ttcaacgtattgatggttcataaacgttcacacacaggggaacgtccccttcagtgcgaaatctgcgggtttacctgccgccaaaaaggcaatttattgcgtcacattaagcttcatactggcgaaaaacccttcaagtgtcatctgtgtaactatgcgtgccagcgtcgcgacgcattagctgaagccgccgccaaagaagctgccgccaaagaggcggccgcgaaggcggactacaaagatcatgacggtgattataaagatcacgatattgactataaggacgacgacgacaaaatggcaccaaagaagaaacgcaaagtcacatgccgctcgcgccctggggagcgtccctttcagtgtcgtatttgcatgcgcaatttttcccgccgccatggactggatcgtcatacacgcacacatacaggagaaaagccattccagtgtcgtatctgtatgcgcaatttctcagaccattccagtcttaagcgtcatctgcgtacccacacgggatctcaaaaaccgttccagtgccgcatctgtatgcgtaacttttcggtacgccataacttaacgcgtcatttacgcacccatacaggggagaagccgtttcagtgccgcatttgcatgcgtaacttctccgatcatagcaatctgagccgccacttgaaaacacatacgggttcgcagaagccgtttcagtgtcgcatttgtatgcgcaatttctcccaacgttcctctttggtacgccacttgcgtacacacacgggggagaaaccattccaatgtcgcatctgtatgcgcaacttttctgagtcagggcatctgaagcgtcacctgcgtactcacctgcgcggttcgggtggaggcggcagtggcggtacgagaaccttggtcactttcaaagatgttttcgtagactttacacgcgaggagtggaagctgctggataccgcacagcaaatcgtgtatcgtaacgtcatgcttgagaactataagaaccttgtcagcttggggtacggtggaggcggcagtggcggtacgatgagcgagcttattaaggagaacatgcatatgaaactgtacatggagggcaccgttgacaaccatcactttaagtgcacatcagaaggcgaaggtaaaccatatgagggcacacaaaccatgcgcatcaaggtcgtggaaggagggcctctgcctttcgccttcgacattttagccacaagctttctgtacggcagcaaaactttcatcaatcatactcaggggatccctgatttctttaaacagtcattccccgagggtttcacttgggagcgtgtcactacctacgaagatggtggagtgttgacagcgactcaggacactagtctgcaggatgggtgtttgatctacaatgtcaaaatccgtggggttaactttacgtccaatggccccgtgatgcagaaaaagacgctggggtgggaggcatttactgagacgctttacccggccgatggaggacttgaaggacgtaacgatatggcactgaagttagtgggtggatctcaccttattgccaatatcaagacaacgtatcgttctaaaaagccagcaaagaacttaaagatgccaggggtgtattatgtggactaccgtttagagcgtattaaggaagcgaataacgagacgtatgtggaacaacatgaggtggccgtggcccgttactgtgacctgccgtcgaaattagggcacaagcttaattgactgcaggcatgcgtgactgactgaggccgcgactctagtttaaacatttatttatttatttatttaacatcggttccctgtttaatatttaaacagtgcggtaagcatttatttatttatttatttaacatcggttccctgtttaatatttaaacaga

SEQ ID NO: 67 - DNA, 762 bp, synthetic construct
  misc_feature 1..762: note = Description of Artificial Sequence: Synthetic polynucleotide
  source 1..762: mol_type = other DNA
cgggtttcgtaacaatcgcatgaggattcgcaacgccttcggcgtagccgatgtcgcgctcccgtctcagtaaaggtcggcgtagccgatgtcgcgcaatcggactgccttcgtacggcgtagccgatgtcgcgcgtatcagtcgcctcggaacggcgtagccgatgtcgcgcattcgtaagaggctcactctcccttacacggagtggataactagtggatctgcgatcgctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctac

SEQ ID NO: 68 - protein, 6 aa, synthetic construct
  REGION 1..6: note = Description of Artificial Sequence: Synthetic peptide
  VARIANT 4: note = X can be any amino acid
  VARIANT 5: note = X can be any amino acid
DSGXXS

(19) United States
(12) Patent Application Publication
(10) Pub. No.: US 2024/0141364 A1
(43) Pub. Date: May 2, 2024

(54) DRUG-REGULATABLE TRANSCRIPTIONAL REPRESSORS

(71) Applicant: Senti Biosciences, Inc., South San Francisco, CA (US)

(72) Inventors: Michelle Elizabeth Hung, South San Francisco, CA (US); Rebecca Tayler Cottman, San Carlos, CA (US); Russell Morrison Gordley, San Francisco, CA (US)

(21) Appl. No.: 18/482,513

(22) Filed: Oct. 6, 2023

Related U.S. Application Data
(63) Continuation of application No. PCT/US2022/023735, filed on Apr. 6, 2022.
(60) Provisional application No. 63/171,551, filed on Apr. 6, 2021.

(51) Int. Cl. C12N 15/63; A61K 31/45; A61K 35/12; A61K 47/64; C07K 14/47

(52) U.S. Cl. CPC C12N 15/635; A61K 31/45; A61K 35/12; A61K 47/64; C07K 14/4703; C07K 2319/81

(57) ABSTRACT

Provided herein are chimeric polypeptides for drug-regulated transcriptional repression. Also provided are methods of inhibiting repression of a gene of interest.

### CROSS REFERENCE TO RELATED APPLICATIONS

This application is a continuation of International Application No. PCT/US2022/023735, filed Apr. 6, 2022, which claims the benefit of U.S. Provisional Application No. 63/171,551, filed Apr. 6, 2021, each of which are hereby incorporated by reference in their entireties for all purposes.

### SEQUENCE LISTING

The instant application contains a Sequence Listing which has been submitted via Patent Center and is hereby incorporated by reference in its entirety. Said XML copy, created on Oct. 26, 2023, is named STB-031W0C1, and is 120,114 bytes in size.

### BACKGROUND

Currently available cell and gene therapy products can lack expression control, which can lead to safety concerns such as toxicity in subjects that receive such therapies. Thus, additional methods of expression control and regulation for these therapies are needed.

### SUMMARY

Provided herein is a chimeric polypeptide comprising (i) an inducible transcription modulator (ITM), wherein the ITM comprises a transcriptional repressor domain and a DNA binding domain; and (ii) a degron, wherein the degron is operably linked to the ITM. In some aspects, the transcriptional repressor domain is selected from the group consisting of: a KRAB repression domain, an HDAC4 domain, a SCX HLH domain, a ID1 HLH domain, a HERC2 Cyt-b5 domain, a TWST1 HLH domain, an NKX22 homeodomain, an ID3 HLH domain, and a TWST2 HLH domain.

The chimeric polypeptide described herein provides a reversible genetic ON-switch by inhibiting transcriptional repression in response to a small molecule drug.

In some aspects, the wherein the transcriptional repressor domain of the chimeric polypeptide comprises the KRAB repression domain. In some aspects, the KRAB repression domain comprises the amino acid sequence of SEQ ID NO: 2. In some aspects, the KRAB repression domain comprises minKRAB. In some aspects, the KRAB repression domain is a KRAB repressor domain variant of SEQ ID NO: 2 and comprises one or more amino acid substitutions selected from the group consisting of: W27L, K28L, D31A, T32A, Q34A, Q35A, R39E, L43S, T57C, K58C, P59C, V61Y, I62Y, I62A, L63W, L63Y, L63E, R64F, R64W, R64E, L65F, L65E, L65W, E66V, K67F, G68F, and E69Fv. In some aspects, the KRAB repression domain is a KRAB repressor domain variant of SEQ ID NO: 2 and comprises one or more amino acid substitutions selected from: Q34A/Q35A, I62A, T57C/K58C/P59C, D31A/T32A, L63W/R64W/L65W, E66V, L63E/R64E/L65E, R64F/L65F, W27L/K28L KRAB, R39E, K67F/G68F/E69F, and V61Y/I62Y/L63Y.

In some aspects, the KRAB repression domain comprises the amino acid sequence of SEQ ID NO: 3. In some aspects, the transcriptional repressor domain comprises the HDAC4 repression domain. In some aspects, the HDAC4 repression domain comprises the amino acid sequence of SEQ ID NO: 4.

In some aspects, the DNA binding domain of the chimeric polypeptide comprises a zinc finger (ZF) protein domain. In some aspects, the ZF protein domain is modular in design and is composed of zinc finger arrays (ZFA). In some aspects, the ZF protein domain comprises one to ten ZFA. In some aspects, the ZF protein domain comprises ten ZFA.

In some aspects, the transcriptional repressor domain is N-terminal to the DNA binding domain. In other aspects, the transcriptional repressor domain is C-terminal to the DNA binding domain.

In some aspects, the transcriptional repressor domain and the DNA binding domain are separated by a first peptide linker. In some aspects, the first peptide linker comprises the amino acid sequence of GGGGSGGT (SEQ ID NO: 60).

In some aspects, the degron of the chimeric polypeptide is selected from the group consisting of: HCV NS4 degron, PEST (two copies of residues 277-307 of human IκBα), GRR (residues 352-408 of human p105), DRR (residues 210-295 of yeast Cdc34), SNS (tandem repeat of SP2 and NB (SP2-NB-SP2 of influenza A or influenza B), RPB (four copies of residues 1688-1702 of yeast RPB), SPmix (tandem repeat of SP1 and SP2 (SP2-SP1-SP2-SP1-SP2 of influenza A virus M2 protein), NS2 (three copies of residues 79-93 of influenza A virus NS protein), ODC (residues 106-142 of ornithine decarboxylase), Nek2A, mouse ODC (residues 422-461), mouse ODC_DA (residues 422-461 of mODC including D433A and D434A point mutations), an APC/C degron, a COP1 E3 ligase binding degron motif, a CRL4-Cdt2 binding PIP degron, an actinfilin-binding degron, a KEAP1 binding degron, a KLHL2 and KLHL3 binding degron, an MDM2 binding motif, an N-degron, a hydroxyproline modification in hypoxia signaling, a phytohormone-dependent SCF-LRR-binding degron, an SCF ubiquitin ligase binding phosphodegron, a phytohormone-dependent SCF-LRR-binding degron, a DSGxxS phospho-dependent degron (“DSGxxS” disclosed as SEQ ID NO: 68), an Siah binding motif, an SPOP SBC docking motif, and a PCNA binding PIP box. In some aspects, the degron comprises a cereblon (CRBN) polypeptide substrate domain capable of binding CRBN in response to an immunomodulatory drug (IMiD) thereby promoting ubiquitin pathway-mediated degradation of the chimeric polypeptide. In some aspects, the CRBN polypeptide substrate domain is selected from the group consisting of: IKZF1, IKZF3, CKla, ZFP91, GSPT1, MEIS2, GSS E4F1, ZN276, ZN517, ZN582, ZN653, ZN654, ZN692, ZN787, and ZN827, or a fragment thereof that is capable of drug-inducible binding of CRBN. In some aspects, the CRBN polypeptide substrate domain is a chimeric fusion product of native CRBN polypeptide sequences. In some aspects, the CRBN polypeptide substrate domain is a IKZF3/ZFP91/IKZF3 chimeric fusion product having the amino acid sequence of FNVLMVHKRSHTGERPLQCEICGFTCRQKGNLLRHIKLHTGEKPFKCHLCNYACQRR DAL (SEQ ID NO: 6). In some aspects, the IMiD is an FDA-approved drug. In some aspects, the IMiD is selected from the group consisting of: thalidomide, lenalidomide, and pomalidomide.

In some aspects, the inducible transcription modulator (ITM) is N-terminal to the degron. In other aspects, the ITM is C-terminal to the degron.

In some aspects, the ITM is separated from the degron by a second peptide linker. In some aspects, the second peptide linker comprises an amino acid sequence selected from the group consisting of: GSGSGSGS (SEQ ID NO: 7), KEGS (SEQ ID NO: 8), EGK, EAAAK (SEQ ID NO: 9), and AAPAKQE (SEQ ID NO: 10). In some embodiments, the second peptide linker comprises an amino acid sequence selected from the group consisting of: AAPAKQEAAAPAKQEAAAPAKQEAAAPAPAAKAEAPAAAPAAKA (SEQ ID NO: 12) and AEAAAKEAAAKEAAAKA (SEQ ID NO: 13).

Also provided is an expression cassette comprising a promoter operably linked to a polynucleotide sequence encoding a chimeric polypeptide as described herein. In some aspects the promoter operably linked to the polynucleotide sequence encoding the chimeric polypeptide comprises a constitutive promoter. In some aspects, the constitutive promoter is selected from the group consisting of: CMV, EFS, SFFV, SV40, MND, PGK, UbC, hEF1a, hCAGG, hACTb, heIF4A1, hGAPDH, hGRP78, hGRP94, hHSP70, hKINb, and hUBIb.

In some aspects, the promoter operably linked to the polynucleotide sequence encoding the chimeric polypeptide comprises an inducible promoter. In some aspects, the inducible promoter comprises a minimal promoter and a response element selected from the group consisting of: NFkB response element, CREB response element, NFAT response element, SRF response element 1, SRF response element 2, AP1 response element, TCF-LEF response element promoter fusion, Hypoxia responsive element, SMAD binding element, STAT3 binding site, inducer molecule responsive promoters, and tandem repeats thereof, NFkB response element, CREB response element, NFAT response element, SRF response element 1, SRF response element 2, AP1 response element, TCF-LEF response element promoter fusion, Hypoxia responsive element, SMAD binding element, STAT3 binding site, inducer molecule responsive promoters, and tandem repeats thereof.

In some aspects, the promoter operably linked to the polynucleotide sequence encoding the chimeric polypeptide is a synthetic promoter.

In some aspects, the polynucleotide sequence encoding the chimeric polypeptide further encodes a 3′untranslated region (UTR) comprising an mRNA-destabilizing element. In some aspects, the mRNA-destabilizing element is selected from the group consisting of: an AU-rich element and a stem-loop destabilizing element.

Also provided is an expression system comprising: (i) an expression cassette comprising a promoter operably linked to a polynucleotide sequence encoding a chimeric polypeptide as described herein, and (ii) a target expression cassette comprising an ITM-responsive promoter operably linked to a gene of interest. In some aspects, the ITM-responsive promoter comprises a promoter sequence and a sequence that binds to the DNA binding domain of the ITM. In some aspects, the sequence that binds to the DNA binding domain comprises one or more zinc finger binding sites. In some aspects, the sequence that binds to the DNA binding domain comprises four of more zinc finger binding sites. In some aspects, the promoter sequence of the ITM-responsive promoter comprises a constitutive promoter sequence. In some aspects, the constitutive promoter sequence is selected from the group consisting of: CMV, EFS, SFFV, SV40, MND, PGK, UbC, EF1a, hCAGG, hACTb, heIF4A1, hGAPDH, hGRP78, hGRP94, hHSP70, hKINb, and hUBIb. In some aspects, the promoter sequence of the ITM-responsive promoter comprises a minimal promoter. In some embodiments, the ITM-responsive promoter comprises a synthetic promoter.

In some embodiments, the gene of interest encodes a therapeutic polypeptide. In some embodiments, the gene of interest encodes a polypeptide selected from the group consisting of: a cytokine, a chemokine, a homing molecule, a growth factor, a cell death regulator, a co-activation molecule, a tumor microenvironment modifier a, a receptor, a ligand, an antibody, a polynucleotide, a peptide, and an enzyme.

In some embodiments, the expression system comprises a heterologous construct that comprises both of: (i) the expression cassette comprising a promoter operably linked to a polynucleotide sequence encoding a chimeric polypeptide as described herein and (ii) the target expression cassette.

In some embodiments, the expression system comprises: (i) a first heterologous construct comprising the expression cassette comprising a promoter operably linked to a polynucleotide sequence encoding a chimeric polypeptide as described herein and (ii) a second heterologous construct comprising the target expression cassette.

Also provided is an isolated cell comprising (i) an expression cassette comprising a promoter operably linked to a polynucleotide sequence encoding a chimeric polypeptide as described herein or (ii) an expression system as described herein. In some embodiments, the isolated cell is a human cell. In some aspects, the isolated cell is a stem cell. In some aspects, the isolated cell is an immune cell. In some aspects, the cell is selected from the group consisting of: a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an erythrocyte, a platelet cell, a human embryonic stem cell (ESC), an ESC-derived cell, a pluripotent stem cell, a mesenchymal stromal cell (MSC), an induced pluripotent stem cell (iPSC), and an iPSC-derived cell.

Also provided is a genetic switch for inhibiting repression of a gene of interest, comprising: a chimeric polypeptide as described herein and a ligand, wherein binding of the ligand to the degron induces degradation of the chimeric polypeptide, thereby inhibiting repression of the gene of interest. In some aspects, the ligand of the genetic switch comprises an immunomodulatory drug (IMiD) that promotes ubiquitin pathway-mediated degradation of the chimeric polypeptide. In some aspects, the IMiD is an FDA-approved drug. In some aspects, the IMiD is selected from the group consisting of: thalidomide, lenalidomide, and pomalidomide.

Also provided is a method of inhibiting repression of a gene of interest, comprising: (a) transforming a cell with an expression system comprising (i) an expression cassette encoding a chimeric polypeptide as described herein, and (ii) a target expression cassette comprising an ITM-responsive promoter operably linked to a gene of interest; (b) culturing the transformed cell under conditions suitable for expression of the chimeric polypeptide; and (c) inducing degradation of the chimeric polypeptide by contacting the transformed cell with a ligand that promotes degradation of the chimeric polypeptide.

### BRIEF DESCRIPTION OF THE SEVERAL VIEWS OF THE DRAWINGS

These and other features, aspects, and advantages of the present disclosure will become better understood with regard to the following description, and accompanying drawings.

FIG. 1 shows the efficiency of degradation of various degron-linked DNA binders following treatment with pomalidomide. Left columns represent “No Drug” and right columns represent “+1 uM Pom.”

FIG. 2 shows induction of reporter expression following degradation of various degron-linked transcriptional repressors. Left columns represent “No Drug” and right columns represent “+1 uM Pom.”

FIG. 3 shows the difference between reporter expression in the absence of pomalidomide as compared to after treatment with pomalidomide, in cells expressing various degron-linked transcriptional repressors.

FIG. 4A shows histogram plots of reporter expression for cells transduced with two degron-linked transcriptional repressor constructs (SB03758 & SB03759, respectively) in the presence or absence of pomalidomide, and as compared to untransduced cells (lacking the reporter), and reporter cells not transduced with a repressor.

FIG. 4B shows histogram plots of reporter expression for cells transduced with two degron-linked transcriptional repressor constructs (SB03758 & SB03759, respectively) in the presence or absence of pomalidomide, and as compared to untransduced cells (lacking the reporter), and reporter cells not transduced with a repressor.

FIG. 5 shows efficiency of degradation of two degron-linked DNA binders each including an mRNA-destabilization tag, following treatment with pomalidomide. Left columns represent “No Drug” and right columns represent “1 uM Pomalidomide.”

FIG. 6A shows reporter expression in the presence or absence of pomalidomide, in cells expressing various degron-linked transcriptional repressors having the degron at the N-terminus. Shown is the fold change in geometric mean fluorescent intensity (gMFI) for reporter expression in cells expressing the reporter and the degron-linked transcriptional repressors, as compared to cells expressing only the reporter but no degron-linked transcriptional repressor. Left columns represent “No Drug” and right columns represent “1 uM Pom.”

FIG. 6B shows reporter expression in the presence or absence of pomalidomide, in cells expressing various degron-linked transcriptional repressors having the degron at the N-terminus. Shown is the fold change in reporter expression for cells expressing each of the degron-linked transcriptional repressors of FIG. 6A, comparing expression in the presence of pomalidomide to expression in the absence of pomalidomide.

FIG. 7A shows reporter expression in the presence or absence of pomalidomide, in cells expressing various degron-linked transcriptional repressors having the degron at the C-terminus. Shown is the fold change in geometric mean fluorescent intensity (gMFI) for reporter expression in cells expressing the reporter and the degron-linked transcriptional repressors, as compared to cells expressing only the reporter but no degron-linked transcriptional repressor. Left columns represent “No Drug” and right columns represent “1 uM Pom.”

FIG. 7B shows reporter expression in the presence or absence of pomalidomide, in cells expressing various degron-linked transcriptional repressors having the degron at the C-terminus. Shown is the fold change in reporter expression for cells expressing each of the degron-linked transcriptional repressors of FIG. 7A, comparing expression in the presence of pomalidomide to expression in the absence of pomalidomide.

FIG. 8 shows IMiD regulated ON switch controls expression of IL-12 in systems including degron-linked repressroes. The top panel shows IL-12 expression levels as regulated by the three indicated degron-linked repressors in drug-free conditions or conditions with a titration of pomalidomide (1 nM, 10 nM, 100 nM, and 1 uM). The bottom panel shows IL-12 levels displayed as a fraction of IL12 expression levels quantified in cells transduced with constitutive IL-12 reporter.

FIG. 9 shows ON to OFF kinetics of IMiD ON switch at 1 uM Pomalidomide and 1 uM Iberdomide. Shown are IL-12 expression levels in supernatant gathered from cells at indicated elapsed time from 1 uM pomalidomide (top panel) or 1 uM Iberdomide (bottom panel) treatment. SB04640 only (right columns) serves as the reporter only control where IL-12 expression is constitutive and not regulated by degron-linked repressor (left columns).

FIG. 10 shows ON to OFF kinetics of IMiD ON switch at 1 uM pomalidomide with additional time points at 12 and 16 hours. IL-12 expression levels in supernatant were gathered from cells at indicated elapsed time from 1 uM pomalidomide treatment. Shown are expression levels of IL-12 normalized to IL-12 expression levels in cells transduced with the reporter only control SB04640 where IL-12 expression is constitutive and not regulated by degron-linked repressor.

### DETAILED DESCRIPTION

Terms used in the claims and specification are defined as set forth below unless otherwise specified.

The term “in vivo” refers to processes that occur in a living organism.

The term “mammal” as used herein includes both humans and non-humans and include but is not limited to humans, non-human primates, canines, felines, murines, bovines, equines, and porcines.

The term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

The term “sufficient amount” means an amount sufficient to produce a desired effect, e.g., an amount sufficient to inhibit transcriptional repression in a cell.

It must be noted that, as used in the specification and the appended claims, the singular forms “a,” “an” and “the” include plural referents unless the context clearly dictates otherwise.

Degradable Transcriptional Repressor Chimeric Polypeptides

The present disclosure provides chimeric polypeptides that include an inducible transcription modulator (ITM) and a degron. In general, the degron is operably linked to the ITM to allow for degron-based degradation of the chimeric polypeptide to regulate the activity of the ITM.

### Inducible Transcription Modulator

The chimeric polypeptides described herein include an inducible transcription modulator (ITM). The ITM includes a DNA binding domain and a transcriptional repressor domain.

In some embodiments, the transcriptional repressor domain can include, but is not limited to, a Krüppel associated box (KRAB) repression domain, a Histone deacetylase 4 (HDAC4) domain, a Scleraxis (SCX) HLH domain, an Inhibitor of DNA binding 1 (ID1) HLH domain, a HECT domain and RCC1-like domain-containing protein 2 (HERC2) Cyt-b5 domain, a Twist-related protein 1 (TWST1) HLH domain, an Homeobox protein Nkx-2.2 (NKX22) homeodomain, an Inhibitor of DNA binding 1 (ID3) HLH domain, or a Twist-related protein 2 (TWST2) HLH domain.

In some embodiments, the transcriptional repressor domain includes a Krüppel associated box (KRAB) repression domain. “KRAB repression domain” as used herein refers to a wild-type, a variant, or a segment of a KRAB domain that is capable of repressing transcription when operably linked to a DNA binding domain. C2H2/Krüppel-type zinc finger (ZNF) proteins are transcription factors that include a KRAB domain and a C-terminal array of zinc fingers that bind to DNA. KRAB domain is made up of canonical subdomain-A (KRAB-A) with or without an auxiliary subdomain, such as KRAB-B, KRAB-BL, KRAB-b or KRAB-C. In some embodiments, the KRAB repressor domain comprises a KRAB domain of the zinc finger protein ZNF10. An exemplary ZNF10 sequence is provided as SEQ ID NO: 1. A KRAB domain of ZNF10 includes residues 2-81 of ZNF10 and is provided as SEQ ID NO: 2. In some embodiments, the KRAB repressor domain comprises the amino acid sequence of SEQ ID NO: 2.

In some embodiments, the KRAB repressor domain comprises minKRAB. “MinKRAB” refers to a 45-amino acid segment of the KRAB repression domain has been identified as a minimal repression domain present within the KRAB repression domain. An exemplary minKRAB repressor domain is provided as SEQ ID NO: 3.

In some embodiments, the KRAB repressor domain comprises a KRAB repressor domain variant. “KRAB repressor domain variant” as used herein refers to a KRAB domain (e.g., SEQ ID NO: 2 or SEQ ID NO: 3) that retains repressor activity but has one or more amino acid substitutions. In some embodiments, the KRAB repressor domain comprises an amino acid sequence of SEQ ID NO: 2 and includes one or more amino acid substitutions selected from: W27L, K28L, D31A, T32A, Q34A, Q35A, R39E, L43S, T57C, K58C, P59C, V61Y, I62Y, I62A, L63W, L63Y, L63E, R64F, R64W, R64E, L65F, L65E, L65W, E66V, K67F, G68F, and E69F.

In some embodiments, the KRAB repression domain includes a KRAB repressor domain variant of SEQ ID NO: 2 and comprises one or more amino acid substitutions selected from: Q34A/Q35A, I62A, T57C/K58C/P59C, D31A/T32A, L63W/R64W/L65W, E66V, L63E/R64E/L65E, R64F/L65F, W27L/K28L KRAB, R39E, K67F/G68F/E69F, and V61Y/I62Y/L63Y.

In some embodiments, the transcriptional repressor domain includes a histone deacetylase 4 (HDAC4) domain. An exemplary HDAC4 domain is provided as SEQ ID NO: 4.

In some embodiments, the DNA binding domain includes a zinc finger (ZF) protein domain. Inclusion of a ZF protein domain allows for targeted nucleic acid binding by the inducible transcription modulator (ITM).

In some embodiments, the ZF protein domain is modular in design and is composed of a zinc finger array (ZFA).

A zinc finger array (ZFA) comprises multiple zinc finger protein motifs that are linked together, optionally separated by flexible linker sequences. Each zinc finger motif binds to a different nucleic acid motif. This results in a ZFA with specificity to any desired nucleic acid sequence. The ZF motifs can be directly adjacent to each other, or separated by a flexible linker sequence. In some embodiments, a ZFA includes an array, string, or chain of ZF motifs arranged in tandem. A ZFA can have 1, 2, 3, 4, 5, 6, 7, 8, 9, 10, 11, 12, 13, 14, or 15 zinc finger motifs. The ZFA can have from 1-10, 1-15, 1-2, 1-3, 1-4, 1-5, 1-6, 1-7, 1-8, 1-9, 2-3, 2-4, 2-5, 2-6, 2-7, 2-8, 2-9, 2-10, 3-4, 3-5 3-6, 3-7, 3-8, 3-9, 3-10, 4-5, 4-6, 4-7, 4-8, 4-9, 4-10, 5-6, 5-7, 5-8, 5-9, 5-10, or 5-15 zinc finger motifs.

The ZF protein domain can have 1, 2, 3, 4, 5, 6, 7, 8, 9, 10, 11, 12, 13, 14, 15, or more ZFAs. The ZF domain can have from 1-10, 1-15, 1-2, 1-3, 1-4, 1-5, 1-6, 1-7, 1-8, 1-9, 2-3, 2-4, 2-5, 2-6, 2-7, 2-8, 2-9, 2-10, 3-4, 3-5 3-6, 3-7, 3-8, 3-9, 3-10, 4-5, 4-6, 4-7, 4-8, 4-9, 4-10, 5-6, 5-7, 5-8, 5-9, 5-10, 5-15, 10-15, 10-20, or 10-25 ZFAs. In some embodiments, the ZF protein domain comprises one to ten ZFA(s). In some embodiments, the ZF protein domain comprises at least one ZFA. In some embodiments, the ZF protein domain comprises at least two ZFAs. In some embodiments, the ZF protein domain comprises at least three ZFAs. In some embodiments, the ZF protein domain comprises at least four ZFAs. In some embodiments, the ZF protein domain comprises at least five ZFAs. In some embodiments, the ZF protein domain comprises at least ten ZFAs.

In some embodiments, the ZFA includes six zinc finger motifs. An exemplary ZF protein domain composed of a ZFA including six zinc finger motifs is shown in the sequence

(SEQ ID NO: 5)
SRPGERPFQCRICMRNFSRRHGLDRHTRTHTGEKPFQCRICMRNFSDHS
SLKRHLRTHTGSQKPFQCRICMRNFSVRHNLTRHLRTHTGEKPFQCRIC
MRNFSDHSNLSRHLKTHTGSQKPFQCRICMRNFSQRSSLVRHLRTHTGE
KPFQCRICMRNFSESGHLKRHLRTHLRGS.

### Degron

In some embodiments, the chimeric polypeptides described herein include a degron. In some embodiments, the degron is capable of inducing degradation of the ITM. In some embodiments, the degron can include, but is not limited to, of HCV NS4 degron, PEST (two copies of residues 277-307 of human IκBα), GRR (residues 352-408 of human p105), DRR (residues 210-295 of yeast Cdc34), SNS (tandem repeat of SP2 and NB (SP2-NB-SP2 of influenza A or influenza B), RPB (four copies of residues 1688-1702 of yeast RPB), SPmix (tandem repeat of SP1 and SP2 (SP2-SP1-SP2-SP1-SP2 of influenza A virus M2 protein), NS2 (three copies of residues 79-93 of influenza A virus NS protein), ODC (residues 106-142 of ornithine decarboxylase), Nek2A, mouse ODC (residues 422-461), mouse ODC_DA (residues 422-461 of mODC including D433A and D434A point mutations), an APC/C degron, a COP1 E3 ligase binding degron motif, a CRL4-Cdt2 binding PIP degron, an actinfilin-binding degron, a KEAP1 binding degron, a KLHL2 and KLHL3 binding degron, an MDM2 binding motif, an N-degron, a hydroxyproline modification in hypoxia signaling, a phytohormone-dependent SCF-LRR-binding degron, an SCF ubiquitin ligase binding phosphodegron, a phytohormone-dependent SCF-LRR-binding degron, a DSGxxS phospho-dependent degron (“DSGxxS” disclosed as SEQ ID NO: 68), an Siah binding motif, an SPOP SBC docking motif, or a PCNA binding PIP box.

In some embodiments, the degron includes a cereblon (CRBN) polypeptide substrate domain capable of binding CRBN in response to an immunomodulatory drug (IMiD) thereby promoting ubiquitin pathway-mediated degradation of the ITM. In some embodiments, the CRBN polypeptide substrate domain can include, but is not limited to, a IKZF1, IKZF3, CKla, ZFP91, GSPT1, MEIS2, GSS E4F1, ZN276, ZN517, ZN582, ZN653, ZN654, ZN692, ZN787, or ZN827, or a fragment thereof that is capable of drug-inducible binding of CRBN. In some embodiments, the CRBN polypeptide substrate domain includes a chimeric fusion product of native CRBN polypeptide sequences. In some embodiments, the CRBN polypeptide substrate domain includes a IKZF3/ZFP91/IKZF3 chimeric fusion product having the amino acid sequence of FNVLMVHKRSHTGERPLQCEICGF TCRQKGNLLRHIKLHTGEKPFKCHLCNYACQRR DAL (SEQ ID NO: 6). Degrons are described in International Application Pub. No. WO2019/089592A1, herein incorporated by reference for all purposes.

### Peptide Linkers

In some embodiments, the chimeric polypeptide includes at least one peptide linker.

A peptide linker can be any polypeptide sequence that separates two polypeptide domains (e.g., an inducible transcription modulator and a degron) without interfering with the function of the polypeptide domains. Examples of peptide linkers include GSGSGSGS (SEQ ID NO: 7), KEGS (SEQ ID NO: 8), EGK, EAAAK (SEQ ID NO: 9), AAPAKQE (SEQ ID NO: 10), GSGSGSGSGGAEAAAKEAAAKEAAAKA (SEQ ID NO: 11, referred to herein as “Concatenated Max Jen linker” or “ConMJ”), AAPAKQEAAAPAKQEAAAPAKQEAAAPAPAAKAEAPAAAPAAKA (SEQ ID NO: 12, referred to herein as “ecpd”), AEAAAKEAAAKEAAAKA (SEQ ID NO: 13), and GGGGSGGT (SEQ ID NO: 60).

In some embodiments, the transcriptional repressor domain and the DNA binding domain are separated by a peptide linker.

In some embodiments, the inducible transcription modulator (ITM) and the degron are separated by a peptide linker. In some embodiments, the peptide linker between the ITM and degron comprises an amino acid sequence selected from: GSGSGSGS (SEQ ID NO: 7), KEGS (SEQ ID NO: 8), EGK, EAAAK (SEQ ID NO: 9), AAPAKQE (SEQ ID NO: 10), and GGGGSGGT (SEQ ID NO: 60). In some embodiments, the peptide linker between the ITM and degron comprises an amino acid sequence GGGGSGGT (SEQ ID NO: 60).

In some embodiments, the peptide linker between the ITM and degron includes an amino acid sequence selected from: GSGSGSGSGGAEAAAKEAAAKEAAAKA (SEQ ID NO: 11, referred to herein as “Concatenated Max Jen linker” or “ConMJ”),

(SEQ ID NO: 12
AAPAKQEAAAPAKQEAAAPAKQEAAAPAPAAKAEAPAAAPAAKA,

referred to herein as “ecpd”), AEAAAKEAAAKEAAAKA (SEQ ID NO: 13), GSGSGSGS (SEQ ID NO: 7), KEGS (SEQ ID NO: 8), and EGK.

In some embodiments, the peptide linker between the ITM and degron includes an amino acid sequence selected from:

(SEQ ID NO: 12, referred to herein as “ecpd”)
AAPAKQEAAAPAKQEAAAPAKQEAAAPAPAAKAEAPAAAPAAKA
and
(SEQ ID NO: 13)
AEAAAKEAAAKEAAAKA.

In some embodiments, the transcriptional repressor domain, the DNA binding domain are separated by a peptide linker, and the degron are each separated by a peptide linker. In some embodiments, the transcriptional repressor domain, the DNA binding domain are separated by a peptide linker, and the degron are each separated by a distinct peptide linker.

Genetic Switches

Also provided herein are genetic switches for inhibiting repression of a gene of interest. A genetic switch may include (a) a chimeric polypeptide including a degron and an inducible transcription modulator that is capable of repressing transcription of a gene of interest, and (b) a ligand that binds to the degron of the chimeric polypeptide and induces degradation of the chimeric polypeptide. Degradation of the chimeric polypeptide releases the repressor activity of the inducible transcription modulator.

In some embodiments, the ligand includes an immunomodulatory drug (IMiD) that promotes ubiquitin pathway-mediated degradation of the chimeric polypeptide.

In some embodiments, the IMiD is an FDA-approved drug.

In some embodiments, the IMiD can include, but is not limited to, thalidomide, lenalidomide, or pomalidomide.

Isolated Polynucleotide Molecules and Expression Cassettes

Also provided herein are polynucleotide molecules (e.g., isolated polynucleotide molecules) and expression cassettes encoding chimeric polypeptides as described herein. In some embodiments the present disclosure provides an expression cassette comprising a promoter operably linked to a polynucleotide sequence encoding the chimeric polypeptide.

“Isolated” nucleic acid molecule or polynucleotide refers to a polynucleotide molecule, such as DNA or RNA, which has been removed from its native environment. For example, a polynucleotide encoding a chimeric polypeptide contained in a heterologous construct is considered isolated. Further examples of an isolated polynucleotide include recombinant polynucleotides maintained in heterologous host cells or purified (partially or substantially) polynucleotides in solution. An isolated polynucleotide also includes a polynucleotide molecule contained in cells that ordinarily contain the polynucleotide molecule, but the polynucleotide molecule is present extrachromosomally or at a chromosomal location that is different from its natural chromosomal location.

Isolated polynucleotide molecules include, but are not limited to a cDNA polynucleotide, an RNA polynucleotide, an RNAi oligonucleotide (e.g., siRNAs, miRNAs, antisense oligonucleotides, shRNAs, etc.), an mRNA polynucleotide, a circular plasmid, a linear DNA fragment, a vector, a minicircle, a ssDNA, a bacterial artificial chromosome (BAC), and yeast artificial chromosome (YAC), and an oligonucleotide.

In some embodiments, the isolated polynucleotide molecule can include, but is not limited to, a DNA, a cDNA, an RNA, an mRNA, and a naked plasmid (linear or circular).

By a nucleic acid or polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These alterations of the reference sequence may occur at the 5′ or 3′ terminal positions of the reference nucleotide sequence or anywhere between those terminal positions, interspersed either individually among residues in the reference sequence or in one or more contiguous groups within the reference sequence. As a practical matter, whether any particular polynucleotide sequence is at least 80%, 85%, 90%, 95%, 96%, 97%, 98% or 99% identical to a nucleotide sequence of the present invention can be determined conventionally using known computer programs.

The term “expression cassette” refers to a polynucleotide generated recombinantly or synthetically, with a series of nucleic acid elements that permit transcription of a particular polynucleotide in a target cell. The expression cassette can be incorporated into a plasmid, chromosome, mitochondrial DNA, plastid DNA, virus, or nucleic acid fragment. Typically, the expression cassette portion of an expression vector includes, among other sequences, a nucleic acid sequence to be transcribed and a promoter.

In some embodiments, the expression cassette including the polynucleotide sequence encoding the chimeric polypeptide further encodes a 3′untranslated region (UTR) comprising an mRNA-destabilizing element that is operably linked to the polynucleotide sequence encoding the chimeric polypeptide. In some embodiments, the mRNA-destabilizing element comprises an AU-rich element and/or a stem-loop destabilizing element (SLDE).

In some embodiments, the mRNA-destabilizing element comprises an AU-rich element. In some embodiments, the AU-rich element includes at least two overlapping motifs of the sequence ATTTA (SEQ ID NO: 14). In some embodiments, the AU-rich element comprises ATTTATTTATTTATTTATTTA (SEQ ID NO: 15).

In some embodiments, the mRNA-destabilizing element comprises a stem-loop destabilizing element (SLDE). In some embodiments, the SLDE comprises

(SEQ ID NO: 16)
CTGTTTAATATTTAAACAG.

In some embodiments, the mRNA-destabilizing element comprises at least one AU-rich element and at least one SLDE. “AuSLIDE” as used herein refers to an AU-rich element operably linked to a stem-loop destabilizing element (SLDE). An exemplary AuSLIDE sequence is provided as SEQ ID NO: 17. In some embodiments, the mRNA-destabilizing element comprises a 2× AuSLIDE. An exemplary AuSLDE sequence is provided as SEQ ID NO: 18.

In some embodiments, the present disclosure provides an expression cassette including a polynucleotide encoding a chimeric polypeptide as described herein. In some embodiments, the present disclosure provides an expression system including a first expression cassette encoding a chimeric polypeptide as previously described, and a target expression cassette comprising an ITM-responsive promoter operably linked to a gene of interest.

“Target expression cassette” refers to an expression cassette including a gene with inducible transcription modulator (ITM)-controllable expression. The expression is controlled by the ITM based on the presence of a ligand (e.g., pomalidomide).

The isolated polynucleotide molecules and heterologous constructs as described herein are engineered polynucleotide molecules. An “engineered polynucleotide” is a polynucleotide that does not occur in nature. It should be understood, however, that while an engineered polynucleotide as a whole is not naturally-occurring, it may include nucleotide sequences that occur in nature. In some embodiments, an engineered polynucleotide comprises nucleotide sequences from different organisms (e.g., from different species). For example, in some embodiments, an engineered polynucleotide includes a murine nucleotide sequence, a bacterial nucleotide sequence, a human nucleotide sequence, and/or a viral nucleotide sequence. The term “engineered polynucleotide” includes recombinant nucleic acids and synthetic nucleic acids. A “recombinant polynucleotide” refers to a molecule that is constructed by joining nucleotide molecules and, in some embodiments, can replicate in a live cell. A “synthetic polynucleotide” refers to a molecule that is amplified or chemically, or by other means, synthesized. Synthetic polynucleotides include those that are chemically modified, or otherwise modified, but can base pair with naturally-occurring nucleotide molecules. Modifications include, but are not limited to, one or more modified internucleotide linkages and non-natural nucleic acids. Modifications are described in further detail in U.S. Pat. No. 6,673,611 and U.S. Application Publication 2004/0019001 and, each of which is incorporated by reference in their entirety. Modified internucleotide linkages can be a phosphorodithioate or phosphorothioate linkage. Non-natural nucleic acids can be a locked nucleic acid (LNA), a peptide nucleic acid (PNA), glycol nucleic acid (GNA), a phosphorodiamidate morpholino oligomer (PMO or “morpholino”), and threose nucleic acid (TNA). Non-natural nucleic acids are described in further detail in International Application WO 1998/039352, U.S. Application Pub. No. 2013/0156849, and U.S. Pat. Nos. 6,670,461; 5,539,082; 5,185,444, each herein incorporated by reference in their entirety. Recombinant polynucleotides and synthetic polynucleotides also include those molecules that result from the replication of either of the foregoing. Engineered polynucleotides of the present disclosure may be encoded by a single molecule (e.g., included in the same plasmid or other vector) or by multiple different molecules (e.g., multiple different independently-replicating molecules).

Engineered polynucleotides of the present disclosure may be produced using standard molecular biology methods (see, e.g., Green and Sambrook, Molecular Cloning, A Laboratory Manual, 2012, Cold Spring Harbor Press). In some embodiments, engineered nucleic acid constructs are produced using GIBSON ASSEMBLY® Cloning (see, e.g., Gibson, D. G. et al. Nature Methods, 343-345, 2009; and Gibson, D. G. et al. Nature Methods, 901-903, 2010, each of which is incorporated by reference herein). GIBSON ASSEMBLY® typically uses three enzymatic activities in a single-tube reaction: 5′ exonuclease, the ‘Y extension activity of a DNA polymerase and DNA ligase activity. The 5’ exonuclease activity chews back the 5′ end sequences and exposes the complementary sequence for annealing. The polymerase activity then fills in the gaps on the annealed regions. A DNA ligase then seals the nick and covalently links the DNA fragments together. The overlapping sequence of adjoining fragments is much longer than those used in Golden Gate Assembly, and therefore results in a higher percentage of correct assemblies. In some embodiments, engineered nucleic acid constructs are produced using IN-FUSION® cloning (Clontech).

### Promoters

As used herein, a “promoter” refers to a control region of a nucleic acid sequence at which initiation and rate of transcription of the remainder of a nucleic acid sequence are controlled. A promoter may also contain sub-regions at which regulatory proteins and molecules may bind, such as RNA polymerase and other transcription factors. Promoters may be constitutive, inducible, repressible, tissue-specific or any combination thereof. A promoter drives expression or drives transcription of the nucleic acid sequence that it regulates. Herein, a promoter is considered to be “operably linked” when it is in a correct functional location and orientation in relation to a nucleic acid sequence it regulates to control (“drive”) transcriptional initiation and/or expression of that sequence.

A promoter may be one naturally associated with a gene or sequence, as may be obtained by isolating the 5′ non-coding sequences located upstream of the coding segment of a given gene or sequence. Such a promoter can be referred to as “endogenous.” In some embodiments, a coding nucleic acid sequence may be positioned under the control of a recombinant or heterologous promoter, which refers to a promoter that is not normally associated with the encoded sequence in its natural environment. Such promoters may include promoters of other genes; promoters isolated from any other cell; and synthetic promoters or enhancers that are not “naturally occurring” such as, for example, those that contain different elements of different transcriptional regulatory regions and/or mutations that alter expression through methods of genetic engineering that are known in the art. In addition to producing nucleic acid sequences of promoters and enhancers synthetically, sequences may be produced using recombinant cloning and/or nucleic acid amplification technology, including polymerase chain reaction (PCR) (see, e.g., U.S. Pat. Nos. 4,683,202 and 5,928,906).

As used herein, an “inducible promoter” refers to a promoter characterized by regulating (e.g., initiating or activating) transcriptional activity when in the presence of, influenced by or contacted by a signal. The signal may be endogenous or a normally exogenous condition (e.g., light), compound (e.g., chemical or non-chemical compound) or protein (e.g., cytokine) that contacts an inducible promoter in such a way as to be active in regulating transcriptional activity from the inducible promoter. Activation of transcription may involve directly acting on a promoter to drive transcription or indirectly acting on a promoter by inactivation of a repressor that is preventing the promoter from driving transcription. Conversely, deactivation of transcription may involve directly acting on a promoter to prevent transcription or indirectly acting on a promoter by activating a repressor that then acts on the promoter.

As used herein, a promoter is “responsive to” or “modulated by” a local tumor state (e.g., inflammation or hypoxia) or signal if in the presence of that state or signal, transcription from the promoter is activated, deactivated, increased, or decreased. In some embodiments, the promoter comprises a response element. A “response element” is a short sequence of DNA within a promoter region that binds specific molecules (e.g., transcription factors) that modulate (regulate) gene expression from the promoter. Response elements that may be used in accordance with the present disclosure include, without limitation, a phloretin-adjustable control element (PEACE), a zinc-finger DNA binding domain (DBD), an interferon-gamma-activated sequence (GAS) (Decker, T. et al. J Interferon Cytokine Res. 1997 March; 17(3):121-34, incorporated herein by reference), an interferon-stimulated response element (ISRE) (Han, K. J. et al. J Biol Chem. 2004 Apr. 9; 279(15):15652-61, incorporated herein by reference), a NF-kappaB response element (Wang, V. et al. Cell Reports. 2012; 2(4): 824-839, incorporated herein by reference), and a STAT3 response element (Zhang, D. et al. J of Biol Chem. 1996; 271: 9503-9509, incorporated herein by reference). Other response elements are encompassed herein. Response elements can also contain tandem repeats (e.g., consecutive repeats of the same nucleotide sequence encoding the response element) to generally increase sensitivity of the response element to its cognate binding molecule. Tandem repeats can be labeled 2×, 3×, 4×, 5×, etc. to denote the number of repeats present.

Non-limiting examples of responsive promoters (also referred to as “inducible promoters”) (e.g., TGF-beta responsive promoters) are listed in Table 1, which shows the design of the promoter and transcription factor, as well as the effect of the inducer molecule towards the transcription factor (TF) and transgene transcription (T) is shown (B, binding; D, dissociation; n.d., not determined) (A, activation; DA, deactivation; DR, derepression) (see Homer, M. & Weber, W. FEBS Letters 586 (2012) 20784-2096m, and references cited therein). Non-limiting examples of components of inducible promoters include those shown in Table 2.

TABLE 1

| | | | | Response to | |
|---|---|---|---|---|---|
| | Promoter and | Transcription | Inducer | inducer | |
| System | operator | factor (TF) | molecule | TF | T |
| Transcriptional activator-responsive promoters | | | | | |
| AIR | PAIR (OalcA-PhCMVmin) | AlcR | Acetaldehyde | n.d. | A |
| ART | PART (OARG-PhCMVmin) | ArgR-VP16 | 1-Arginine | B | A |
| BIT | PBIT3 (OBirA3-PhCMVmin) | BIT (BirA-VP16) | Biotin | B | A |
| Cumate - | PCR5 (OCuO6-PhCMVmin) | cTA (CymR-VP16) | Cumate | D | DA |
| activator | | | | | |
| Cumate - | PCR5 (OCuO6-PhCMVmin) | rcTA (rCymR-VP16) | Cumate | B | A |
| reverse activator | | | | | |
| E-OFF | PETR (OETR-PhCMVmin) | ET (E-VP16) | Erythromycin | D | DA |
| NICE-OFF | PNIC (ONIC-PhCMVmin) | NT (HdnoR-VP16) | 6-Hydroxy-nicotine | D | DA |
| PEACE | PTtgR1 (OTtgR-PhCMVmin) | TtgA1 (TtgR-VP16) | Phloretin | D | DA |
| PIP-OFF | PPIR (OPIR-Phsp70min) | PIT (PIP-VP16) | Pristinamycin I | D | DA |
| QuoRex | PSCA (OscbR-PhCMVmin)PSPA | SCA (ScbR-VP16) | SCB1 | D | DA |
| | (OpapRI-PhCMVmin) | | | | |
| Redox | PROP (OROP-PhCMVmin) | REDOX (REX-VP16) | NADH | D | DA |
| TET-OFF | PhCMV*-1 | tTA (TetR-VP16) | Tetracycline | D | DA |
| | (OtetO7-PhCMVmin) | | | | |
| TET-ON | PhCMV*-1 | rtTA (rTetR-VP16) | Doxycycline | B | A |
| | (OtetO7-PhCMVmin) | | | | |
| TIGR | PCTA (OrheO-PhCMVmin) | CTA (RheA-VP16) | Heat | D | DA |
| TraR | O7x(tra box)-PhCMVmin | p65-TraR | 3-Oxo-C8-HSL | B | A |
| VAC-OFF | P1VanO2 (OVanO2-PhCMVmin) | VanA1 (VanR-VP16) | Vanillic acid | D | DA |
| Transcriptional repressor-responsive promoters | | | | | |
| Cumate - | PCuO (PCMV5-OCuO) | CymR | Cumate | D | DR |
| repressor | | | | | |
| E-ON | PETRON8 (PSV40-OETR8) | E-KRAB | Erythromycin | D | DR |
| NICE-ON | PNIC (PSV40-ONIC8) | NS (HdnoR-KRAB) | 6-Hydroxy-nicotine | D | DR |
| PIP-ON | PPIRON (PSV40-OPIR3) | PIT3 (PIP-KRAB) | Pristinamycin I | D | DR |
| Q-ON | PSCAON8 (PSV40-OscbR8) | SCS (ScbR-KRAB) | SCB1 | D | DR |
| TET-ON<comma> | OtetO-PHPRT | tTS-H4 (TetR-HDAC4) | Doxycycline | D | DR |
| repressor-based | | | | | |
| T-REX | PTetO (PhCMV-OtetO2) | TetR | Tetracycline | D | DR |
| UREX | PUREX8 (PSV40-OhucO8) | mUTS (KRAB-HucR) | Uric acid | D | DR |
| VAC-ON | PVanON8 (PhCMV-OVanO8) | VanA4 (VanR-KRAB) | Vanillic acid | D | DR |
| Hybrid promoters | | | | | |
| QuoRexPIP- | OscbR8-OPIR3- | SCAPIT3 | SCB1Pristinamycin I | DD | DADR |
| ON(NOT IF gate) | PhCMVmin | | | | |
| QuoRexE- | OscbR-OETR8- | SCAE-KRAB | SCB1Erythromycin | DD | DADR |
| ON(NOT IF gate) | PhCMVmin | | | | |
| TET-OFFE- | OtetO7-OETR8- | tTAE-KRAB | Tetracycline | DD | DADR |
| ON(NOT IF gate) | PhCMVmin | | Erythromycin | | |
| TET-OFFPIP- | OtetO7-OPIR3- | tTAPIT3E-KRAB | Tetracycline | DDD | DADR |
| ONE-ON | OETR8-PhCMVmin | | Pristinamycin | | DR |
| | | | IErythromycin | | |

TABLE 2

| Name | DNA SEQUENCE | Source |
|---|---|---|
| minimal promoter; minP | AGAGGGTATATAATGGAAGCTCGAC | EU581860.1 |
| | TTCCAG (SEQ ID NO: 19) | (Promega) |
| NFKB response element | GGGAATTTCCGGGGACTTTCCGGGA | EU581860.1 |
| protein promoter; 5x | ATTTCCGGGGACTTTCCGGGAATTTC | (Promega) |
| NFKB-RE | C (SEQ ID NO: 20) | |
| CREB response element | CACCAGACAGTGACGTCAGCTGCCA | DQ904461.1 |
| protein promoter; 4x | GATCCCATGGCCGTCATACTGTGAC | (Promega) |
| CRE | GTCTTTCAGACACCCCATTGACGTCA | |
| | ATGGGAGAA (SEQ ID NO: 21) | |
| NFAT response element | GGAGGAAAAACTGTTTCATACAGAA | DQ904462.1 |
| protein promoter; 3x | GGCGTGGAGGAAAAACTGTTTCATA | (Promega) |
| NFAT binding sites | CAGAAGGCGTGGAGGAAAAACTGTT | |
| | TCATACAGAAGGCGT (SEQ ID NO: | |
| | 22) | |
| SRF response element | AGGATGTCCATATTAGGACATCTAG | FJ773212.1 |
| protein promoter; 5x | GATGTCCATATTAGGACATCTAGGA | (Promega) |
| SRE | TGTCCATATTAGGACATCTAGGATGT | |
| | CCATATTAGGACATCTAGGATGTCC | |
| | ATATTAGGACATCT (SEQ ID NO: 23) | |
| SRF response element | AGTATGTCCATATTAGGACATCTACC | FJ773213.1 |
| protein promoter 2; 5x | ATGTCCATATTAGGACATCTACTATG | (Promega) |
| SRF-RE | TCCATATTAGGACATCTTGTATGTCC | |
| | ATATTAGGACATCTAAAATGTCCAT | |
| | ATTAGGACATCT (SEQ ID NO: 24) | |
| AP1 response element | TGAGTCAGTGACTCAGTGAGTCAGT | JQ858516.1 |
| protein promoter; 6x | GACTCAGTGAGTCAGTGACTCAG | (Promega) |
| AP1-RE | (SEQ ID NO: 25) | |
| TCF-LEF response | AGATCAAAGGGTTTAAGATCAAAGG | JX099537.1 |
| element promoter; 8x | GCTTAAGATCAAAGGGTATAAGATC | (Promega) |
| TCF-LEF-RE | AAAGGGCCTAAGATCAAAGGGACTA | |
| | AGATCAAAGGGTTTAAGATCAAAGG | |
| | GCTTAAGATCAAAGGGCCTA (SEQ ID | |
| | NO: 26) | |
| SBEx4 | GTCTAGACGTCTAGACGTCTAGACG | Addgene Cat No: 16495 |
| | TCTAGAC (SEQ ID NO: 27) | |
| SMAD2/3-CAGACA | CAGACACAGACACAGACACAGACA | Jonk et al. (J Biol |
| x4 | (SEQ ID NO: 28) | Chem. 1998 Aug |
| | | 14;273(33):21145-52. |
| STAT3 binding site | Ggatccggtactcgagatctgcgatctaagtaagcttggca | Addgene Sequencing |
| | ttccggtactgttggtaaagccac (SEQ ID NO: 29) | Result #211335 |

As used herein, a “constitutive promoter” refers to a promoter that allows for continual or un-regulated transcriptional activity.

Non-limiting examples of constitutive promoters include the cytomegalovirus (CMV) promoter, the elongation factor 1-alpha (EF1a) promoter and EF1a variants hEF1aV1 and hEF1aV2, the elongation factor short (EFS) promoter, the MND promoter (a synthetic promoter that contains the U3 region of a modified MoMuLV LTR with myeloproliferative sarcoma virus enhancer), the phosphoglycerate kinase (PGK) promoter, the spleen focus-forming virus (SFFV) promoter, the simian virus 40 (SV40) promoter, and the ubiquitin C (UbC) promoter. Examples of constitutive promoter amino acid sequences are shown in Table 3.

TABLE 3

| Name | DNA SEQUENCE |
|---|---|
| CMV | GTTGACATTGATTATTGACTAGTTATTAATAGTAATCAATTACGGGGTCAT |
| | TAGTTCATAGCCCATATATGGAGTTCCGCGTTACATAACTTACGGTAAATG |
| | GCCCGCCTGGCTGACCGCCCAACGACCCCCGCCCATTGACGTCAATAATG |
| | ACGTATGTTCCCATAGTAACGCCAATAGGGACTTTCCATTGACGTCAATGG |
| | GTGGAGTATTTACGGTAAACTGCCCACTTGGCAGTACATCAAGTGTATCAT |
| | ATGCCAAGTACGCCCCCTATTGACGTCAATGACGGTAAATGGCCCGCCTG |
| | GCATTATGCCCAGTACATGACCTTATGGGACTTTCCTACTTGGCAGTACAT |
| | CTACGTATTAGTCATCGCTATTACCATGGTGATGCGGTTTTGGCAGTACAT |
| | CAATGGGCGTGGATAGCGGTTTGACTCACGGGGATTTCCAAGTCTCCACC |
| | CCATTGACGTCAATGGGAGTTTGTTTTGGCACCAAAATCAACGGGACTTTC |
| | CAAAATGTCGTAACAACTCCGCCCCATTGACGCAAATGGGCGGTAGGCGT |
| | GTACGGTGGGAGGTCTATATAAGCAGAGCTC (SEQ ID NO: 30) |
| EF1a | GGCTCCGGTGCCCGTCAGTGGGCAGAGCGCACATCGCCCACAGTCCCCGA |
| | GAAGTTGGGGGGAGGGGTCGGCAATTGAACCGGTGCCTAGAGAAGGTGG |
| | CGCGGGGTAAACTGGGAAAGTGATGCCGTGTACTGGCTCCGCCTTTTTCCC |
| | GAGGGTGGGGGAGAACCGTATATAAGTGCAGTAGTCGCCGTGAACGTTCT |
| | TTTTCGCAACGGGTTTGCCGCCAGAACACAGGTAAGTGCCGTGTGTGGTTC |
| | CCGCGGGCCTGGCCTCTTTACGGGTTATGGCCCTTGCGTGCCTTGAATTAC |
| | TTCCACCTGGCTGCAGTACGTGATTCTTGATCCCGAGCTTCGGGTTGGAAG |
| | TGGGTGGGAGAGTTCGAGGCCTTGCGCTTAAGGAGCCCCTTCGCCTCGTG |
| | CTTGAGTTGAGGCCTGGCCTGGGCGCTGGGGCCGCCGCGTGCGAATCTGG |
| | TGGCACCTTCGCGCCTGTCTCGCTGCTTTCGATAAGTCTCTAGCCATTTAA |
| | AATTTTTGATGACCTGCTGCGACGCTTTTTTTCTGGCAAGATAGTCTTGTA |
| | AATGCGGGCCAAGATCTGCACACTGGTATTTCGGTTTTTGGGGCCGCGGG |
| | CGGCGACGGGGCCCGTGCGTCCCAGCGCACATGTTCGGCGAGGCGGGGCC |
| | TGCGAGCGCGACCACCGAGAATCGGACGGGGGTAGTCTCAAGCTGGCCG |
| | GCCTGCTCTGGTGCCTGTCCTCGCGCCGCCGTGTATCGCCCCGCCCCGGGC |
| | GGCAAGGCTGGCCCGGTCGGCACCAGTTGCGTGAGCGGAAAGATGGCCG |
| | CTTCCCGGTCCTGCTGCAGGGAGCTCAAAATGGAGGACGCGGCGCTCGGG |
| | AGAGCGGGCGGGTGAGTCACCCACACAAAGGAAAAGGGCCTTTCCGTCCT |
| | CAGCCGTCGCTTCATGTGACTCCACGGAGTACCGGGCGCCGTCCAGGCAC |
| | CTCGATTAGTTCTCGAGCTTTTGGAGTACGTCGTCTTTAGGTTGGGGGGAG |
| | GGGTTTTATGCGATGGAGTTTCCCCACACTGAGTGGGTGGAGACTGAAGT |
| | TAGGCCAGCTTGGCACTTGATGTAATTCTCCTTGGAATTTGCCCTTTTTGA |
| | GTTTGGATCTTGGTTCATTCTCAAGCCTCAGACAGTGGTTCAAAGTTTTTTT |
| | CTTCCATTTCAGGTGTCGTGA (SEQ ID NO: 31) |
| EFS | GGATCTGCGATCGCTCCGGTGCCCGTCAGTGGGCAGAGCGCACATCGCCC |
| | ACAGTCCCCGAGAAGTTGGGGGGAGGGGTCGGCAATTGAACCGGTGCCTA |
| | GAGAAGGTGGCGCGGGGTAAACTGGGAAAGTGATGTCGTGTACTGGCTCC |
| | GCCTTTTTCCCGAGGGTGGGGGAGAACCGTATATAAGTGCAGTAGTCGCC |
| | GTGAACGTTCTTTTTCGCAACGGGTTTGCCGCCAGAACACAGCTGAAGCTT |
| | CGAGGGGCTCGCATCTCTCCTTCACGCGCCCGCCGCCCTACCTGAGGCCGC |
| | CATCCACGCCGGTTGAGTCGCGTTCTGCCGCCTCCCGCCTGTGGTGCCTCC |
| | TGAACTGCGTCCGCCGTCTAGGTAAGTTTAAAGCTCAGGTCGAGACCGGG |
| | CCTTTGTCCGGCGCTCCCTTGGAGCCTACCTAGACTCAGCCGGCTCTCCAC |
| | GCTTTGCCTGACCCTGCTTGCTCAACTCTACGTCTTTGTTTCGTTTTCTGTT |
| | CTGCGCCGTTACAGATCCAAGCTGTGACCGGCGCCTAC (SEQ ID NO: 32) |
| MND | TTTATTTAGTCTCCAGAAAAAGGGGGGAATGAAAGACCCCACCTGTAGGT |
| | TTGGCAAGCTAGGATCAAGGTTAGGAACAGAGAGACAGCAGAATATGGG |
| | CCAAACAGGATATCTGTGGTAAGCAGTTCCTGCCCCGGCTCAGGGCCAAG |
| | AACAGTTGGAACAGCAGAATATGGGCCAAACAGGATATCTGTGGTAAGC |
| | AGTTCCTGCCCCGGCTCAGGGCCAAGAACAGATGGTCCCCAGATGCGGTC |
| | CCGCCCTCAGCAGTTTCTAGAGAACCATCAGATGTTTCCAGGGTGCCCCA |
| | AGGACCTGAAATGACCCTGTGCCTTATTTGAACTAACCAATCAGTTCGCTT |
| | CTCGCTTCTGTTCGCGCGCTTCTGCTCCCCGAGCTCAATAAAAGAGCCCA |
| | (SEQ ID NO: 33) |
| PGK | GGGGTTGGGGTTGCGCCTTTTCCAAGGCAGCCCTGGGTTTGCGCAGGGAC |
| | GCGGCTGCTCTGGGCGTGGTTCCGGGAAACGCAGCGGCGCCGACCCTGGG |
| | TCTCGCACATTCTTCACGTCCGTTCGCAGCGTCACCCGGATCTTCGCCGCT |
| | ACCCTTGTGGGCCCCCCGGCGACGCTTCCTGCTCCGCCCCTAAGTCGGGAA |
| | GGTTCCTTGCGGTTCGCGGCGTGCCGGACGTGACAAACGGAAGCCGCACG |
| | TCTCACTAGTACCCTCGCAGACGGACAGCGCCAGGGAGCAATGGCAGCGC |
| | GCCGACCGCGATGGGCTGTGGCCAATAGCGGCTGCTCAGCGGGGCGCGCC |
| | GAGAGCAGCGGCCGGGAAGGGGCGGTGCGGGAGGCGGGGTGTGGGGCGG |
| | TAGTGTGGGCCCTGTTCCTGCCCGCGCGGTGTTCCGCATTCTGCAAGCCTC |
| | CGGAGCGCACGTCGGCAGTCGGCTCCCTCGTTGACCGAATCACCGACCTC |
| | TCTCCCCAG (SEQ ID NO: 34) |
| SFFV | GTAACGCCATTTTGCAAGGCATGGAAAAATACCAAACCAAGAATAGAGA |
| | AGTTCAGATCAAGGGCGGGTACATGAAAATAGCTAACGTTGGGCCAAACA |
| | GGATATCTGCGGTGAGCAGTTTCGGCCCCGGCCCGGGGCCAAGAACAGAT |
| | GGTCACCGCAGTTTCGGCCCCGGCCCGAGGCCAAGAACAGATGGTCCCCA |
| | GATATGGCCCAACCCTCAGCAGTTTCTTAAGACCCATCAGATGTTTCCAGG |
| | CTCCCCCAAGGACCTGAAATGACCCTGCGCCTTATTTGAATTAACCAATCA |
| | GCCTGCTTCTCGCTTCTGTTCGCGCGCTTCTGCTTCCCGAGCTCTATAAAA |
| | GAGCTCACAACCCCTCACTCGGCGCGCCAGTCCTCCGACAGACTGAGTCG |
| | CCCGGG (SEQ ID NO: 35) |
| SV40 | CTGTGGAATGTGTGTCAGTTAGGGTGTGGAAAGTCCCCAGGCTCCCCAGC |
| | AGGCAGAAGTATGCAAAGCATGCATCTCAATTAGTCAGCAACCAGGTGTG |
| | GAAAGTCCCCAGGCTCCCCAGCAGGCAGAAGTATGCAAAGCATGCATCTC |
| | AATTAGTCAGCAACCATAGTCCCGCCCCTAACTCCGCCCATCCCGCCCCTA |
| | ACTCCGCCCAGTTCCGCCCATTCTCCGCCCCATGGCTGACTAATTTTTTTTA |
| | TTTATGCAGAGGCCGAGGCCGCCTCTGCCTCTGAGCTATTCCAGAAGTAGT |
| | GAGGAGGCTTTTTTGGAGGCCTAGGCTTTTGCAAAAAGCT (SEQ ID NO: 36) |
| UbC | GCGCCGGGTTTTGGCGCCTCCCGCGGGCGCCCCCCTCCTCACGGCGAGCG |
| | CTGCCACGTCAGACGAAGGGCGCAGGAGCGTTCCTGATCCTTCCGCCCGG |
| | ACGCTCAGGACAGCGGCCCGCTGCTCATAAGACTCGGCCTTAGAACCCCA |
| | GTATCAGCAGAAGGACATTTTAGGACGGGACTTGGGTGACTCTAGGGCAC |
| | TGGTTTTCTTTCCAGAGAGCGGAACAGGCGAGGAAAAGTAGTCCCTTCTC |
| | GGCGATTCTGCGGAGGGATCTCCGTGGGGCGGTGAACGCCGATGATTATA |
| | TAAGGACGCGCCGGGTGTGGCACAGCTAGTTCCGTCGCAGCCGGGATTTG |
| | GGTCGCGGTTCTTGTTTGTGGATCGCTGTGATCGTCACTTGGTGAGTTGCG |
| | GGCTGCTGGGCTGGCCGGGGCTTTCGTGGCCGCCGGGCCGCTCGGTGGGA |
| | CGGAAGCGTGTGGAGAGACCGCCAAGGGCTGTAGTCTGGGTCCGCGAGC |
| | AAGGTTGCCCTGAACTGGGGGTTGGGGGGAGCGCACAAAATGGCGGCTGT |
| | TCCCGAGTCTTGAATGGAAGACGCTTGTAAGGCGGGCTGTGAGGTCGTTG |
| | AAACAAGGTGGGGGGCATGGTGGGCGGCAAGAACCCAAGGTCTTGAGGC |
| | CTTCGCTAATGCGGGAAAGCTCTTATTCGGGTGAGATGGGCTGGGGCACC |
| | ATCTGGGGACCCTGACGTGAAGTTTGTCACTGACTGGAGAACTCGGGTTT |
| | GTCGTCTGGTTGCGGGGGCGGCAGTTATGCGGTGCCGTTGGGCAGTGCAC |
| | CCGTACCTTTGGGAGCGCGCGCCTCGTCGTGTCGTGACGTCACCCGTTCTG |
| | TTGGCTTATAATGCAGGGTGGGGCCACCTGCCGGTAGGTGTGCGGTAGGC |
| | TTTTCTCCGTCGCAGGACGCAGGGTTCGGGCCTAGGGTAGGCTCTCCTGAA |
| | TCGACAGGCGCCGGACCTCTGGTGAGGGGAGGGATAAGTGAGGCGTCAG |
| | TTTCTTTGGTCGGTTTTATGTACCTATCTTCTTAAGTAGCTGAAGCTCCGGT |
| | TTTGAACTATGCGCTCGGGGTTGGCGAGTGTGTTTTGTGAAGTTTTTTAGG |
| | CACCTTTTGAAATGTAATCATTTGGGTCAATATGTAATTTTCAGTGTTAGA |
| | CTAGTAAAGCTTCTGCAGGTCGACTCTAGAAAATTGTCCGCTAAATTCTGG |
| | CCGTTTTTGGCTTTTTTGTTAGAC (SEQ ID NO: 37) |
| hEF1aV1 | GGCTCCGGTGCCCGTCAGTGGGCAGAGCGCACATCGCCCACAGTCCCCGA |
| | GAAGTTGGGGGGAGGGGTCGGCAATTGAACCGGTGCCTAGAGAAGGTGG |
| | CGCGGGGTAAACTGGGAAAGTGATGTCGTGTACTGGCTCCGCCTTTTTCCC |
| | GAGGGTGGGGGAGAACCGTATATAAGTGCAGTAGTCGCCGTGAACGTTCT |
| | TTTTCGCAACGGGTTTGCCGCCAGAACACAGGTAAGTGCCGTGTGTGGTTC |
| | CCGCGGGCCTGGCCTCTTTACGGGTTATGGCCCTTGCGTGCCTTGAATTAC |
| | TTCCACCTGGCTGCAGTACGTGATTCTTGATCCCGAGCTTCGGGTTGGAAG |
| | TGGGTGGGAGAGTTCGAGGCCTTGCGCTTAAGGAGCCCCTTCGCCTCGTG |
| | CTTGAGTTGAGGCCTGGCCTGGGCGCTGGGGCCGCCGCGTGCGAATCTGG |
| | TGGCACCTTCGCGCCTGTCTCGCTGCTTTCGATAAGTCTCTAGCCATTTAA |
| | AATTTTTGATGACCTGCTGCGACGCTTTTTTTCTGGCAAGATAGTCTTGTA |
| | AATGCGGGCCAAGATCTGCACACTGGTATTTCGGTTTTTGGGGCCGCGGG |
| | CGGCGACGGGGCCCGTGCGTCCCAGCGCACATGTTCGGCGAGGCGGGGCC |
| | TGCGAGCGCGGCCACCGAGAATCGGACGGGGGTAGTCTCAAGCTGGCCG |
| | GCCTGCTCTGGTGCCTGGTCTCGCGCCGCCGTGTATCGCCCCGCCCTGGGC |
| | GGCAAGGCTGGCCCGGTCGGCACCAGTTGCGTGAGCGGAAAGATGGCCG |
| | CTTCCCGGCCCTGCTGCAGGGAGCTCAAAATGGAGGACGCGGCGCTCGGG |
| | AGAGCGGGCGGGTGAGTCACCCACACAAAGGAAAAGGGCCTTTCCGTCCT |
| | CAGCCGTCGCTTCATGTGACTCCACGGAGTACCGGGCGCCGTCCAGGCAC |
| | CTCGATTAGTTCTCGAGCTTTTGGAGTACGTCGTCTTTAGGTTGGGGGGAG |
| | GGGTTTTATGCGATGGAGTTTCCCCACACTGAGTGGGTGGAGACTGAAGT |
| | TAGGCCAGCTTGGCACTTGATGTAATTCTCCTTGGAATTTGCCCTTTTTGA |
| | GTTTGGATCTTGGTTCATTCTCAAGCCTCAGACAGTGGTTCAAAGTTTTTTT |
| | CTTCCATTTCAGGTGTCGTGA (SEQ ID NO: 38) |
| hCAGG | ACTAGTTATTAATAGTAATCAATTACGGGGTCATTAGTTCATAGCCCATAT |
| | ATGGAGTTCCGCGTTACATAACTTACGGTAAATGGCCCGCCTGGCTGACC |
| | GCCCAACGACCCCCGCCCATTGACGTCAATAATGACGTATGTTCCCATAGT |
| | AACGCCAATAGGGACTTTCCATTGACGTCAATGGGTGGAGTATTTACGGT |
| | AAACTGCCCACTTGGCAGTACATCAAGTGTATCATATGCCAAGTACGCCC |
| | CCTATTGACGTCAATGACGGTAAATGGCCCGCCTGGCATTATGCCCAGTA |
| | CATGACCTTATGGGACTTTCCTACTTGGCAGTACATCTACGTATTAGTCAT |
| | CGCTATTACCATGGTCGAGGTGAGCCCCACGTTCTGCTTCACTCTCCCCAT |
| | CTCCCCCCCCTCCCCACCCCCAATTTTGTATTTATTTATTTTTTAATTATTTT |
| | GTGCAGCGATGGGGGCGGGGGGGGGGGGGGGGCGCGCGCCAGGCGGGGC |
| | GGGGCGGGGCGAGGGGCGGGGCGGGGCGAGGCGGAGAGGTGCGGCGGC |
| | AGCCAATCAGAGCGGCGCGCTCCGAAAGTTTCCTTTTATGGCGAGGCGGC |
| | GGCGGCGGCGGCCCTATAAAAAGCGAAGCGCGCGGCGGGCGGGGAGTCG |
| | CTGCGACGCTGCCTTCGCCCCGTGCCCCGCTCCGCCGCCGCCTCGCGCCGC |
| | CCGCCCCGGCTCTGACTGACCGCGTTACTCCCACAGGTGAGCGGGCGGGA |
| | CGGCCCTTCTCCTCCGGGCTGTAATTAGCGCTTGGTTTAATGACGGCTTGT |
| | TTCTTTTCTGTGGCTGCGTGAAAGCCTTGAGGGGCTCCGGGAGGGCCCTTT |
| | GTGCGGGGGGAGCGGCTCGGGGGGTGCGTGCGTGTGTGTGTGCGTGGGGA |
| | GCGCCGCGTGCGGCTCCGCGCTGCCCGGCGGCTGTGAGCGCTGCGGGCGC |
| | GGCGCGGGGCTTTGTGCGCTCCGCAGTGTGCGCGAGGGGAGCGCGGCCGG |
| | GGGCGGTGCCCCGCGGTGCGGGGGGGGCTGCGAGGGGAACAAAGGCTGC |
| | GTGCGGGGTGTGTGCGTGGGGGGGTGAGCAGGGGGTGTGGGCGCGTCGG |
| | TCGGGCTGCAACCCCCCCTGCACCCCCCTCCCCGAGTTGCTGAGCACGGCC |
| | CGGCTTCGGGTGCGGGGCTCCGTACGGGGCGTGGCGCGGGGCTCGCCGTG |
| | CCGGGCGGGGGGTGGCGGCAGGTGGGGGTGCCGGGCGGGGCGGGGCCGC |
| | CTCGGGCCGGGGAGGGCTCGGGGGAGGGGCGCGGCGGCCCCCGGAGCGC |
| | CGGCGGCTGTCGAGGCGCGGCGAGCCGCAGCCATTGCCTTTTATGGTAAT |
| | CGTGCGAGAGGGCGCAGGGACTTCCTTTGTCCCAAATCTGTGCGGAGCCG |
| | AAATCTGGGAGGCGCCGCCGCACCCCCTCTAGCGGGCGCGGGGCGAAGC |
| | GGTGCGGCGCCGGCAGGAAGGAAATGGGCGGGGAGGGCCTTCGTGCGTC |
| | GCCGCGCCGCCGTCCCCTTCTCCCTCTCCAGCCTCGGGGCTGTCCGCGGGG |
| | GGACGGCTGCCTTCGGGGGGGACGGGGCAGGGCGGGGTTCGGCTTCTGGC |
| | GTGTGACCGGCGGCTCTAGAGCCTCTGCTAACCATGTTCATGCCTTCTTCT |
| | TTTTCCTACAGCTCCTGGGCAACGTGCTGGTTATTGTGCTGTCTCATCATTT |
| | TGGCAAAGAATTC (SEQ ID NO: 39) |
| hEF1aV2 | GGGCAGAGCGCACATCGCCCACAGTCCCCGAGAAGTTGGGGGGAGGGGT |
| | CGGCAATTGAACCGGTGCCTAGAGAAGGTGGCGCGGGGTAAACTGGGAA |
| | AGTGATGTCGTGTACTGGCTCCGCCTTTTTCCCGAGGGTGGGGGAGAACC |
| | GTATATAAGTGCAGTAGTCGCCGTGAACGTTCTTTTTCGCAACGGGTTTGC |
| | CGCCAGAACACAG (SEQ ID NO: 40) |
| hACTb | CCACTAGTTCCATGTCCTTATATGGACTCATCTTTGCCTATTGCGACACAC |
| | ACTCAATGAACACCTACTACGCGCTGCAAAGAGCCCCGCAGGCCTGAGGT |
| | GCCCCCACCTCACCACTCTTCCTATTTTTGTGTAAAAATCCAGCTTCTTGTC |
| | ACCACCTCCAAGGAGGGGGAGGAGGAGGAAGGCAGGTTCCTCTAGGCTG |
| | AGCCGAATGCCCCTCTGTGGTCCCACGCCACTGATCGCTGCATGCCCACCA |
| | CCTGGGTACACACAGTCTGTGATTCCCGGAGCAGAACGGACCCTGCCCAC |
| | CCGGTCTTGTGTGCTACTCAGTGGACAGACCCAAGGCAAGAAAGGGTGAC |
| | AAGGACAGGGTCTTCCCAGGCTGGCTTTGAGTTCCTAGCACCGCCCCGCC |
| | CCCAATCCTCTGTGGCACATGGAGTCTTGGTCCCCAGAGTCCCCCAGCGGC |
| | CTCCAGATGGTCTGGGAGGGCAGTTCAGCTGTGGCTGCGCATAGCAGACA |
| | TACAACGGACGGTGGGCCCAGACCCAGGCTGTGTAGACCCAGCCCCCCCG |
| | CCCCGCAGTGCCTAGGTCACCCACTAACGCCCCAGGCCTGGTCTTGGCTG |
| | GGCGTGACTGTTACCCTCAAAAGCAGGCAGCTCCAGGGTAAAAGGTGCCC |
| | TGCCCTGTAGAGCCCACCTTCCTTCCCAGGGCTGCGGCTGGGTAGGTTTGT |
| | AGCCTTCATCACGGGCCACCTCCAGCCACTGGACCGCTGGCCCCTGCCCTG |
| | TCCTGGGGAGTGTGGTCCTGCGACTTCTAAGTGGCCGCAAGCCACCTGAC |
| | TCCCCCAACACCACACTCTACCTCTCAAGCCCAGGTCTCTCCCTAGTGACC |
| | CACCCAGCACATTTAGCTAGCTGAGCCCCACAGCCAGAGGTCCTCAGGCC |
| | CTGCTTTCAGGGCAGTTGCTCTGAAGTCGGCAAGGGGGAGTGACTGCCTG |
| | GCCACTCCATGCCCTCCAAGAGCTCCTTCTGCAGGAGCGTACAGAACCCA |
| | GGGCCCTGGCACCCGTGCAGACCCTGGCCCACCCCACCTGGGCGCTCAGT |
| | GCCCAAGAGATGTCCACACCTAGGATGTCCCGCGGTGGGTGGGGGGCCCG |
| | AGAGACGGGCAGGCCGGGGGCAGGCCTGGCCATGCGGGGCCGAACCGGG |
| | CACTGCCCAGCGTGGGGCGCGGGGGCCACGGCGCGCGCCCCCAGCCCCCG |
| | GGCCCAGCACCCCAAGGCGGCCAACGCCAAAACTCTCCCTCCTCCTCTTCC |
| | TCAATCTCGCTCTCGCTCTTTTTTTTTTTCGCAAAAGGAGGGGAGAGGGGG |
| | TAAAAAAATGCTGCACTGTGCGGCGAAGCCGGTGAGTGAGCGGCGCGGG |
| | GCCAATCAGCGTGCGCCGTTCCGAAAGTTGCCTTTTATGGCTCGAGCGGCC |
| | GCGGCGGCGCCCTATAAAACCCAGCGGCGCGACGCGCCACCACCGCCGA |
| | GACCGCGTCCGCCCCGCGAGCACAGAGCCTCGCCTTTGCCGATCCGCCGC |
| | CCGTCCACACCCGCCGCCAGGTAAGCCCGGCCAGCCGACCGGGGCAGGCG |
| | GCTCACGGCCCGGCCGCAGGCGGCCGCGGCCCCTTCGCCCGTGCAGAGCC |
| | GCCGTCTGGGCCGCAGCGGGGGGCGCATGGGGGGGGAACCGGACCGCCG |
| | TGGGGGGCGCGGGAGAAGCCCCTGGGCCTCCGGAGATGGGGGACACCCC |
| | ACGCCAGTTCGGAGGCGCGAGGCCGCGCTCGGGAGGCGCGCTCCGGGGG |
| | TGCCGCTCTCGGGGCGGGGGCAACCGGCGGGGTCTTTGTCTGAGCCGGGC |
| | TCTTGCCAATGGGGATCGCAGGGTGGGCGCGGCGGAGCCCCCGCCAGGCC |
| | CGGTGGGGGCTGGGGCGCCATTGCGCGTGCGCGCTGGTCCTTTGGGCGCT |
| | AACTGCGTGCGCGCTGGGAATTGGCGCTAATTGCGCGTGCGCGCTGGGAC |
| | TCAAGGCGCTAACTGCGCGTGCGTTCTGGGGCCCGGGGTGCCGCGGCCTG |
| | GGCTGGGGCGAAGGCGGGCTCGGCCGGAAGGGGTGGGGTCGCCGCGGCT |
| | CCCGGGCGCTTGCGCGCACTTCCTGCCCGAGCCGCTGGCCGCCCGAGGGT |
| | GTGGCCGCTGCGTGCGCGCGCGCCGACCCGGCGCTGTTTGAACCGGGCGG |
| | AGGCGGGGCTGGCGCCCGGTTGGGAGGGGGTTGGGGCCTGGCTTCCTGCC |
| | GCGCGCCGCGGGGACGCCTCCGACCAGTGTTTGCCTTTTATGGTAATAAC |
| | GCGGCCGGCCCGGCTTCCTTTGTCCCCAATCTGGGCGCGCGCCGGCGCCCC |
| | CTGGCGGCCTAAGGACTCGGCGCGCCGGAAGTGGCCAGGGCGGGGGCGA |
| | CCTCGGCTCACAGCGCGCCCGGCTAT (SEQ ID NO: 41) |
| heIF4A1 | GTTGATTTCCTTCATCCCTGGCACACGTCCAGGCAGTGTCGAATCCATCTC |
| | TGCTACAGGGGAAAACAAATAACATTTGAGTCCAGTGGAGACCGGGAGC |
| | AGAAGTAAAGGGAAGTGATAACCCCCAGAGCCCGGAAGCCTCTGGAGGC |
| | TGAGACCTCGCCCCCCTTGCGTGATAGGGCCTACGGAGCCACATGACCAA |
| | GGCACTGTCGCCTCCGCACGTGTGAGAGTGCAGGGCCCCAAGATGGCTGC |
| | CAGGCCTCGAGGCCTGACTCTTCTATGTCACTTCCGTACCGGCGAGAAAG |
| | GCGGGCCCTCCAGCCAATGAGGCTGCGGGGGGGGCCTTCACCTTGATAGG |
| | CACTCGAGTTATCCAATGGTGCCTGCGGGCCGGAGCGACTAGGAACTAAC |
| | GTCATGCCGAGTTGCTGAGCGCCGGCAGGCGGGGCCGGGGCGGCCAAAC |
| | CAATGCGATGGCCGGGGCGGAGTCGGGCGCTCTATAAGTTGTCGATAGGC |
| | GGGCACTCCGCCCTAGTTTCTAAGGACCATG (SEQ ID NO: 42) |
| hGAPDH | AGTTCCCCAACTTTCCCGCCTCTCAGCCTTTGAAAGAAAGAAAGGGGAGG |
| | GGGCAGGCCGCGTGCAGTCGCGAGCGGTGCTGGGCTCCGGCTCCAATTCC |
| | CCATCTCAGTCGCTCCCAAAGTCCTTCTGTTTCATCCAAGCGTGTAAGGGT |
| | CCCCGTCCTTGACTCCCTAGTGTCCTGCTGCCCACAGTCCAGTCCTGGGAA |
| | CCAGCACCGATCACCTCCCATCGGGCCAATCTCAGTCCCTTCCCCCCTACG |
| | TCGGGGCCCACACGCTCGGTGCGTGCCCAGTTGAACCAGGCGGCTGCGGA |
| | AAAAAAAAAGCGGGGAGAAAGTAGGGCCCGGCTACTAGCGGTTTTACGG |
| | GCGCACGTAGCTCAGGCCTCAAGACCTTGGGCTGGGACTGGCTGAGCCTG |
| | GCGGGAGGCGGGGTCCGAGTCACCGCCTGCCGCCGCGCCCCCGGTTTCTA |
| | TAAATTGAGCCCGCAGCCTCCCGCTTCGCTCTCTGCTCCTCCTGTTCGACA |
| | GTCAGCCGCATCTTCTTTTGCGTCGCCAGGTGAAGACGGGCGGAGAGAAA |
| | CCCGGGAGGCTAGGGACGGCCTGAAGGCGGCAGGGGGGGGCGCAGGCCG |
| | GATGTGTTCGCGCCGCTGCGGGGTGGGCCCGGGCGGCCTCCGCATTGCAG |
| | GGGCGGGCGGAGGACGTGATGCGGCGCGGGCTGGGCATGGAGGCCTGGT |
| | GGGGGAGGGGAGGGGAGGCGTGGGTGTCGGCCGGGGCCACTAGGCGCTC |
| | ACTGTTCTCTCCCTCCGCGCAGCCGAGCCACATCGCTGAGACAC (SEQ ID |
| | NO: 43) |
| hGRP78 | AGTGCGGTTACCAGCGGAAATGCCTCGGGGTCAGAAGTCGCAGGAGAGA |
| | TAGACAGCTGCTGAACCAATGGGACCAGCGGATGGGGCGGATGTTATCTA |
| | CCATTGGTGAACGTTAGAAACGAATAGCAGCCAATGAATCAGCTGGGGGG |
| | GCGGAGCAGTGACGTTTATTGCGGAGGGGGCCGCTTCGAATCGGCGGCGG |
| | CCAGCTTGGTGGCCTGGGCCAATGAACGGCCTCCAACGAGCAGGGCCTTC |
| | ACCAATCGGCGGCCTCCACGACGGGGCTGGGGGAGGGTATATAAGCCGA |
| | GTAGGCGACGGTGAGGTCGACGCCGGCCAAGACAGCACAGACAGATTGA |
| | CCTATTGGGGTGTTTCGCGAGTGTGAGAGGGAAGCGCCGCGGCCTGTATT |
| | TCTAGACCTGCCCTTCGCCTGGTTCGTGGCGCCTTGTGACCCCGGGCCCCT |
| | GCCGCCTGCAAGTCGGAAATTGCGCTGTGCTCCTGTGCTACGGCCTGTGGC |
| | TGGACTGCCTGCTGCTGCCCAACTGGCTGGCAC (SEQ ID NO: 44) |
| hGRP94 | TAGTTTCATCACCACCGCCACCCCCCCGCCCCCCCGCCATCTGAAAGGGTT |
| | CTAGGGGATTTGCAACCTCTCTCGTGTGTTTCTTCTTTCCGAGAAGCGCCG |
| | CCACACGAGAAAGCTGGCCGCGAAAGTCGTGCTGGAATCACTTCCAACGA |
| | AACCCCAGGCATAGATGGGAAAGGGTGAAGAACACGTTGCCATGGCTAC |
| | CGTTTCCCCGGTCACGGAATAAACGCTCTCTAGGATCCGGAAGTAGTTCC |
| | GCCGCGACCTCTCTAAAAGGATGGATGTGTTCTCTGCTTACATTCATTGGA |
| | CGTTTTCCCTTAGAGGCCAAGGCCGCCCAGGCAAAGGGGCGGTCCCACGC |
| | GTGAGGGGCCCGCGGAGCCATTTGATTGGAGAAAAGCTGCAAACCCTGAC |
| | CAATCGGAAGGAGCCACGCTTCGGGCATCGGTCACCGCACCTGGACAGCT |
| | CCGATTGGTGGACTTCCGCCCCCCCTCACGAATCCTCATTGGGTGCCGTGG |
| | GTGCGTGGTGCGGCGCGATTGGTGGGTTCATGTTTCCCGTCCCCCGCCCGC |
| | GAGAAGTGGGGGTGAAAAGCGGCCCGACCTGCTTGGGGTGTAGTGGGCG |
| | GACCGCGCGGCTGGAGGTGTGAGGATCCGAACCCAGGGGTGGGGGGTGG |
| | AGGCGGCTCCTGCGATCGAAGGGGACTTGAGACTCACCGGCCGCACGTC |
| | (SEQ ID NO: 45) |
| hHSP70 | GGGCCGCCCACTCCCCCTTCCTCTCAGGGTCCCTGTCCCCTCCAGTGAATC |
| | CCAGAAGACTCTGGAGAGTTCTGAGCAGGGGGCGGCACTCTGGCCTCTGA |
| | TTGGTCCAAGGAAGGCTGGGGGGCAGGACGGGAGGCGAAAACCCTGGAA |
| | TATTCCCGACCTGGCAGCCTCATCGAGCTCGGTGATTGGCTCAGAAGGGA |
| | AAAGGCGGGTCTCCGTGACGACTTATAAAAGCCCAGGGGCAAGCGGTCCG |
| | GATAACGGCTAGCCTGAGGAGCTGCTGCGACAGTCCACTACCTTTTTCGA |
| | GAGTGACTCCCGTTGTCCCAAGGCTTCCCAGAGCGAACCTGTGCGGCTGC |
| | AGGCACCGGCGCGTCGAGTTTCCGGCGTCCGGAAGGACCGAGCTCTTCTC |
| | GCGGATCCAGTGTTCCGTTTCCAGCCCCCAATCTCAGAGCGGAGCCGACA |
| | GAGAGCAGGGAACCC (SEQ ID NO: 46) |
| hKINb | GCCCCACCCCCGTCCGCGTTACAACCGGGAGGCCCGCTGGGTCCTGCACC |
| | GTCACCCTCCTCCCTGTGACCGCCCACCTGATACCCAAACAACTTTCTCGC |
| | CCCTCCAGTCCCCAGCTCGCCGAGCGCTTGCGGGGAGCCACCCAGCCTCA |
| | GTTTCCCCAGCCCCGGGCGGGGCGAGGGGCGATGACGTCATGCCGGCGCG |
| | CGGCATTGTGGGGCGGGGCGAGGCGGGGCGCCGGGGGGAGCAACACTGA |
| | GACGCCATTTTCGGCGGCGGGAGCGGCGCAGGCGGCCGAGCGGGACTGG |
| | CTGGGTCGGCTGGGCTGCTGGTGCGAGGAGCCGCGGGGCTGTGCTCGGCG |
| | GCCAAGGGGACAGCGCGTGGGTGGCCGAGGATGCTGCGGGGCGGTAGCT |
| | CCGGCGCCCCTCGCTGGTGACTGCTGCGCCGTGCCTCACACAGCCGAGGC |
| | GGGCTCGGCGCACAGTCGCTGCTCCGCGCTCGCGCCCGGCGGCGCTCCAG |
| | GTGCTGACAGCGCGAGAGAGCGCGGCCTCAGGAGCAACAC (SEQ ID NO: |
| | 47) |
| hUBIb | TTCCAGAGCTTTCGAGGAAGGTTTCTTCAACTCAAATTCATCCGCCTGATA |
| | ATTTTCTTATATTTTCCTAAAGAAGGAAGAGAAGCGCATAGAGGAGAAGG |
| | GAAATAATTTTTTAGGAGCCTTTCTTACGGCTATGAGGAATTTGGGGCTCA |
| | GTTGAAAAGCCTAAACTGCCTCTCGGGAGGTTGGGCGCGGCGAACTACTT |
| | TCAGCGGCGCACGGAGACGGCGTCTACGTGAGGGGTGATAAGTGACGCA |
| | ACACTCGTTGCATAAATTTGCGCTCCGCCAGCCCGGAGCATTTAGGGGCG |
| | GTTGGCTTTGTTGGGTGAGCTTGTTTGTGTCCCTGTGGGTGGACGTGGTTG |
| | GTGATTGGCAGGATCCTGGTATCCGCTAACAGGTACTGGCCCACAGCCGT |
| | AAAGACCTGCGGGGGCGTGAGAGGGGGGAATGGGTGAGGTCAAGCTGGA |
| | GGCTTCTTGGGGTTGGGTGGGCCGCTGAGGGGAGGGGAGGGCGAGGTGA |
| | CGCGACACCCGGCCTTTCTGGGAGAGTGGGCCTTGTTGACCTAAGGGGGG |
| | CGAGGGCAGTTGGCACGCGCACGCGCCGACAGAAACTAACAGACATTAA |
| | CCAACAGCGATTCCGTCGCGTTTACTTGGGAGGAAGGCGGAAAAGAGGTA |
| | GTTTGTGTGGCTTCTGGAAACCCTAAATTTGGAATCCCAGTATGAGAATGG |
| | TGTCCCTTCTTGTGTTTCAATGGGATTTTTACTTCGCGAGTCTTGTGGGTTT |
| | GGTTTTGTTTTCAGTTTGCCTAACACCGTGCTTAGGTTTGAGGCAGATTGG |
| | AGTTCGGTCGGGGGAGTTTGAATATCCGGAACAGTTAGTGGGGAAAGCTG |
| | TGGACGCTTGGTAAGAGAGCGCTCTGGATTTTCCGCTGTTGACGTTGAAAC |
| | CTTGAATGACGAATTTCGTATTAAGTGACTTAGCCTTGTAAAATTGAGGGG |
| | AGGCTTGCGGAATATTAACGTATTTAAGGCATTTTGAAGGAATAGTTGCT |
| | AATTTTGAAGAATATTAGGTGTAAAAGCAAGAAATACAATGATCCTGAGG |
| | TGACACGCTTATGTTTTACTTTTAAACTAGGTCACC (SEQ ID NO: 48) |

### Promoter Operably Linked to Polynucleotide Encoding a Chimeric Polypeptide

In some embodiments, the present disclosure provides a heterologous construct comprising a promoter operably linked to a polynucleotide sequence encoding a chimeric polypeptide as described herein.

In some embodiments, the promoter operably linked to a polynucleotide sequence encoding the chimeric polypeptide includes a constitutive promoter, an inducible promoter, and/or a synthetic promoter.

In some embodiments, the promoter operatively linked to a polynucleotide encoding the chimeric polypeptide includes a constitutive promoter. Examples of constitutive promoters are shown in Table 3. In some embodiments, the constitutive promoter can include, but is not limited to, CMV, EFS, SFFV, SV40, MND, PGK, UbC, EF1a (e.g., wild-type or a variant such as hEF1aV1 or hEF1aV2), hCAGG, hACTb, heIF4A1, hGAPDH, hGRP78, hGRP94, hHSP70, hKINb, and hUBIb.

### Heterologous Constructs

In some embodiments, the polynucleotide molecules as described herein are included in a heterologous construct. The term “vector” or “expression vector” is synonymous with “heterologous construct” and refers to a polynucleotide molecule that is used to introduce and direct the expression of one or more genes that are operably associated with the construct in a target cell. The term includes the construct as a self-replicating nucleic acid structure as well as the vector incorporated into the genome of a host cell into which it has been introduced. A heterologous construct as described herein includes an expression cassette. In some embodiments, provided herein is a heterologous construct comprising an expression cassette that comprises a polynucleotide molecule that encodes a chimeric polypeptide as described herein. In some embodiments, the heterologous construct further includes a target expression cassette including an inducible transcription modulator-responsive (ITM-responsive) promoter. In some embodiments, provided herein is a first heterologous construct comprising an expression cassette that comprises a polynucleotide molecule encoding the chimeric polypeptide, and a second heterologous construct comprising a target expression cassette including an inducible transcription modulator-responsive (ITM-responsive) promoter.

Expression Systems Including an Inducible Transcription Modulator-Responsive Promoter

In some embodiments, provided herein are expression systems including a first expression cassette encoding a chimeric polypeptide as described herein and a target expression cassette including an inducible transcription modulator-responsive (ITM-responsive) promoter operably linked to a gene of interest.

### Gene of Interest

In some embodiments, the gene of interest includes a therapeutic protein. A therapeutic protein is any polypeptide that when provided to a subject provides a clinical benefit. A therapeutic protein may be provided by administering a polypeptide, administering a cell capable of expressing the polypeptide, or administering a polynucleotide encoding the polypeptide.

In some embodiments, the gene of interest encodes a polypeptide selected from: a cytokine, a chemokine, a homing molecule, a growth factor, a cell death regulator, a co-activation molecule, a tumor microenvironment modifier a, a receptor, a ligand, an antibody, a peptide, and an enzyme.

In some embodiments, the gene of interest includes a cytokine. In some embodiments, the cytokine can include, but is not limited to, IL1-beta, IL2, IL4, IL6, IL7, IL10, IL12, an IL12p70 fusion protein, IL15, IL17A, IL18, IL21, IL22, Type I interferons, Interferon-gamma, and TNF-alpha.

In some embodiments, the gene of interest includes a cell death regulator. Cell death regulators include cell death-inducing polypeptides and cell survival polypeptides.

In some embodiments, the gene of interest includes a cell death-inducing polypeptide. Examples of cell death-inducing polypeptides include: caspase 3, caspase 6, caspase 7, caspase 8, caspase 9, Diphtheria toxin fragment A (DTA), Bax, Bak, Bok, Bad, Bcl-xS, Bak, Bik, Bcl-2-interacting protein 3 (BNIP3), Fas, Fas-associated protein with death domain (FADD), tumor necrosis factor receptor type 1-associated death domain protein (TRADD), a TNF receptor (TNF-β), APAF-1, granzyme B, second mitochondria-derived activator of caspases (SMAC), Omi, Bmf, Bid, Bim, p53-upregulated modulator of apoptosis (PUMA), Noxa, Blk, Hrk, Cytochrome c, Arts, TNF-related cell death-inducing ligand (TRAIL), Herpes Simplex Virus thymidine kinase (HSV-TK), Varicella Zoster Virus thymidine kinase (VZV-TK), viral Spike protein, Carboxyl esterase, cytosine deaminase, nitroreductase Fksb, Carboxypeptidase G2, Carboxypeptidase A, Horseradish peroxidase, Linamarase, Hepatic cytochrome P450-2B1, and Purine nucleoside phosphorylase. In some embodiments, the cell death-inducing polypeptide is caspase 9 or a functional truncation thereof. In some embodiments, the cell death-inducing polypeptide comprises the caspase 9 amino acid sequence of SEQ ID NO: 49. In some embodiments, the cell death-inducing polypeptide is Diphtheria toxin fragment A (DTA). In some embodiments, the cell death-inducing polypeptide comprises the DTA amino acid sequence of SEQ ID NO: 50. In some embodiments, the cell death-inducing polypeptide is granzyme B. In some embodiments, the cell death-inducing polypeptide comprises the granzyme B amino acid sequence of SEQ ID NO: 51. In some embodiments, the cell death-inducing polypeptide is Bax. In some embodiments, the cell death-inducing polypeptide comprises the Bax amino acid sequence of SEQ ID NO: 52.

In some embodiments, the gene of interest includes a cell survival polypeptide. Examples of cell survival polypeptides include: XIAP, Bcl-2, Bcl-xL, Bcl-w, Bcl-2-related protein A1 (BCL2A1), Mc1-1, FLICE-like inhibitory protein (c-FLIP), and an adenoviral E1B-19K protein. In some embodiments, the cell survival polypeptide is XIAP. In some embodiments, the cell survival polypeptide comprises the XIAP amino acid sequence of SEQ ID NO: 53.

### Inducible Transcription Modulator-Responsive Promoter

In some embodiments, the present disclosure provides polynucleotide molecules encoding a gene of interest operably linked to an inducible transcription modulator-responsive promoter (ITM-responsive promoter). In some embodiments, ITM-responsive promoters are synthetic promoters that are responsive to a chimeric polypeptide including an ITM, and repression of the gene of interest by the ITM can be controlled by the presence of a ligand such as pomalidomide.

In some embodiments, the ITM-responsive promoter comprises a promoter sequence and an ITM-binding domain that is specifically recognized by an inducible transcription modulator (ITM) as described herein. “Core promoter sequence” as used herein refers to a portion of a promoter including a core (i.e., “minimal”) promoter sequence that interacts with RNA polymerase II and is sufficient to initiate transcription. In some embodiments, the ITM-responsive promoter includes a synthetic promoter including a core promoter sequence (as provided by the promoter sequence) and a transcription modulator-responsive sequence (as provided by the binding domain) that do not co-occur within a promoter region naturally.

The binding domain may include one or more zinc finger binding sites. A zinc finger binding site is a polynucleotide sequence that is capable of binding to a zinc finger protein domain (e.g., the zinc finger protein domain of SEQ ID NO: 5). The binding domain can comprise 1, 2, 3, 4, 5, 6 7, 8, 9, 10, or more zinc finger binding sites. An exemplary zinc finger binding site comprises GGCGTAGCCGATGTCGCG (SEQ ID NO: 54). In some embodiments, the binding domain comprises one zinc finger binding site. In some embodiments, the binding domain comprises more than one zinc finger binding site. Zinc finger binding sites may be separated by a DNA linker. The DNA linker may be, in some embodiments, 5-40, 5-30, 10-40, 10-30 base pairs in length. In some embodiments, the binding domain comprises two zinc finger binding sites. In some embodiments, the binding domain comprises three zinc finger binding sites. In some embodiments, the binding domain comprises four zinc finger binding sites. An exemplary binding domain comprising zinc finger binding sites is shown in the sequence: cgggtttcgtaacaatcgcatgaggattcgcaacgcctteGGCGTAGCCGATGTCGCGctcccgtctcagtaaaggtc GGCGTAGCCGATGTCGCGcaatcggactgccttcgtacGGCGTAGCCGATGTCGCGcgtatcagtcg cctcggaacGGCGTAGCCGATGTCGCG (SEQ ID NO: 55. The binding domain of SEQ ID NO: 55 includes four binding sites that each bind to a zinc finger protein domain of SEQ ID NO: 5, with each of the binding sites separated by a DNA linker.

In some embodiments, the core promoter sequence includes a minimal promoter. In some embodiments, the core promoter sequence is derived from a promoter selected from: minP, minCMV, YB TATA, and minTK. An exemplary core promoter sequence comprises

(SEQ ID NO: 56)
TCTAGAGGGTATATAATGGGGGCCA.

In some embodiments, the core promoter sequence comprises a sequence of a constitutive promoter. Examples of constitutive promoter sequences are shown in Table 3. In some embodiments, the constitutive promoter sequence can include, but is not limited to, CMV, EFS, SFFV, SV40, MND, PGK, UbC, EF1a, hCAGG, hACTb, heIF4A1, hGAPDH, hGRP78, hGRP94, hHSP70, hKINb, and hUBIb.

An exemplary ITM-responsive promoter includes the sequence:

(SEQ ID NO: 57)
cgggtttcgtaacaatcgcatgaggattcgcaacgccttcGGCGT
AGCCGATGTCGCGctcccgtctcagtaaaggtcGGCGTAGCCGAT
GTCGCGcaatcggactgccttcgtacGGCGTAGCCGATGTCGCGc
gtatcagtcgcctcggaacGGCGTAGCCGATGTCGCGcattcgta
agaggctcactctcccttacacggagtggataACTAGTTCTAGAG
GGTATATAATGGGGGCCA.

Another exemplary ITM-responsive promoter includes the sequence:

(SEQ ID NO: 67)
cgggtttcgtaacaatcgcatgaggattcgcaacgccttcGGCGT
AGCCGATGTCGCGctcccgtctcagtaaaggtcGGCGTAGCCGAT
GTCGCGcaatcggactgccttcgtacGGCGTAGCCGATGTCGCGc
gtatcagtcgcctcggaacGGCGTAGCCGATGTCGCGcattcgta
agaggctcactctcccttacacggagtggataACTAGTGGATCTG
CGATCGCTCCGGTGCCCGTCAGTGGGCAGAGCGCACATCGCCCAC
AGTCCCCGAGAAGTTGGGGGGAGGGGTCGGCAATTGAACCGGTGC
CTAGAGAAGGTGGCGCGGGGTAAACTGGGAAAGTGATGTCGTGTA
CTGGCTCCGCCTTTTTCCCGAGGGTGGGGGAGAACCGTATATAAG
TGCAGTAGTCGCCGTGAACGTTCTTTTTCGCAACGGGTTTGCCGC
CAGAACACAGCTGAAGCTTCGAGGGGCTCGCATCTCTCCTTCACG
CGCCCGCCGCCCTACCTGAGGCCGCCATCCACGCCGGTTGAGTCG
CGTTCTGCCGCCTCCCGCCTGTGGTGCCTCCTGAACTGCGTCCGC
CGTCTAGGTAAGTTTAAAGCTCAGGTCGAGACCGGGCCTTTGTCC
GGCGCTCCCTTGGAGCCTACCTAGACTCAGCCGGCTCTCCACGCT
TTGCCTGACCCTGCTTGCTCAACTCTACGTCTTTGTTTCGTTTTC
TGTTCTGCGCCGTTACAGATCCAAGCTGTGACCGGCGCCTAC.

### Multicistronic and Multiple Promoter Systems

In some embodiments, engineered polynucleotides or constructs of the present disclosure are configured to produce multiple polypeptides. For example, polynucleotides may be configured to produce two different polypeptides. The polynucleotide molecule may be configured to produce a polypeptide including a chimeric protein as described herein and a polypeptide of interest, which expressed under control of a promoter that is responsive to the chimeric protein.

In some embodiments, a chimeric polypeptide as described herein and a gene of interest that can be transcriptionally repressed by the chimeric polypeptide may be encoded by the same polynucleotide molecule or heterologous construct.

In some embodiments, engineered nucleic acids can be multicistronic, i.e., more than one separate polypeptide (e.g., multiple exogenous polynucleotides or effector molecules) can be produced from a single transcript. Engineered nucleic acids can be multicistronic through the use of various linkers, e.g., a polynucleotide sequence encoding a first exogenous polynucleotide can be linked to a nucleotide sequence encoding a second exogenous polynucleotide, such as in a first gene:linker:second gene 5′ to 3′ orientation. A linker polynucleotide sequence can encode one or more 2A ribosome skipping elements, such as T2A. Other 2A ribosome skipping elements include, but are not limited to, E2A, P2A, and F2A. 2A ribosome skipping elements allow production of separate polypeptides encoded by the first and second genes are produced during translation. A linker can encode a cleavable linker polypeptide sequence, such as a Furin cleavage site or a TEV cleavage site, wherein following expression the cleavable linker polypeptide is cleaved such that separate polypeptides encoded by the first and second genes are produced. A cleavable linker can include a polypeptide sequence, such as such a flexible linker (e.g., a Gly-Ser-Gly sequence), that further promotes cleavage.

A linker can encode an Internal Ribosome Entry Site (IRES), such that separate polypeptides encoded by the first and second genes are produced during translation. A linker can encode a splice acceptor, such as a viral splice acceptor.

A linker can be a combination of linkers, such as a Furin-2A linker that can produce separate polypeptides through 2A ribosome skipping followed by further cleavage of the Furin site to allow for complete removal of 2A residues. In some embodiments, a combination of linkers can include a Furin sequence, a flexible linker, and 2A linker. Accordingly, in some embodiments, the linker includes a Furin-Gly-Ser-Gly-2A fusion polypeptide. In some embodiments, a linker includes a Furin-Gly-Ser-Gly-T2A fusion polypeptide.

In general, a multicistronic system can use any number or combination of linkers, to express any number of genes or portions thereof (e.g., an engineered nucleic acid can encode a first, a second, and a third effector molecule, each separated by linkers such that separate polypeptides encoded by the first, second, and third effector molecules are produced).

“Linkers,” as used herein, can refer to peptide linkers that link a first polypeptide sequence and a second polypeptide sequence or the multicistronic linkers described above.

### Post-Transcriptional Regulatory Elements

In some embodiments, an engineered polynucleotide molecule of the present disclosure comprises a post-transcriptional regulatory element (PRE). PREs can modulate RNA stability, for example by destabilizing RNA (e.g., an AU-slide as previously described) or by stabilizing RNA. In some embodiments, PREs can enhance gene expression via enabling tertiary RNA structure stability and 3′ end formation. Non-limiting examples of PREs include the Hepatitis B virus PRE (HPRE) and the Woodchuck Hepatitis Virus PRE (WPRE). In some embodiments, the post-transcriptional regulatory element includes a Woodchuck Hepatitis Virus Posttranscriptional Regulatory Element (WPRE). In some embodiments, the WPRE comprises the alpha, beta, and gamma components of the WPRE element. In some embodiments, the WPRE comprises the alpha component of the WPRE element. Examples of WPRE sequences include SEQ ID NO: 58 and SEQ ID NO: 59.

### Engineered Cells

Also provided herein are cells, and methods of producing cells, that comprise one or more polynucleotide molecules, expression cassettes, or constructs of the present disclosure. These cells are referred to herein as “engineered cells.” These cells, which typically contain one or more engineered nucleic acids, do not occur in nature. In some embodiments, the cells are isolated cells that recombinantly express the one or more engineered polynucleotides. In some embodiments, the engineered polynucleotides are expressed from one or more vectors or a selected locus from the genome of the cell. In some embodiments, the cells are engineered to include a polynucleotide comprising a promoter operably linked to a nucleotide sequence.

An engineered cell of the present disclosure can comprise one or more engineered polynucleotides (e.g., expression systems including inducible transcription modulator (ITM)-responsive promoters) integrated into the cell's genome. An engineered cell can comprise one or more engineered polynucleotide capable of expression without integrating into the cell's genome, for example, engineered with a transient expression system such as a plasmid or mRNA.

### Engineered Cell Types

An engineered cell of the present disclosure can be a human cell. An engineered cell can be a human primary cell. An engineered primary cell can be any somatic cell. An engineered primary cell can be any stem cell. In some embodiments, the engineered cell is derived from the subject. In some embodiments, the engineered cell is allogeneic with reference to the subject.

An engineered cell of the present disclosure can be isolated from a subject, such as a subject known or suspected to have cancer. Cell isolation methods are known to those skilled in the art and include, but are not limited to, sorting techniques based on cell-surface marker expression, such as FACS sorting, positive isolation techniques, and negative isolation, magnetic isolation, and combinations thereof. An engineered cell can be allogenic with reference to the subject being administered a treatment. Allogenic modified cells can be HLA-matched to the subject being administered a treatment. An engineered cell can be a cultured cell, such as an ex vivo cultured cell. An engineered cell can be an ex vivo cultured cell, such as a primary cell isolated from a subject. Cultured cell can be cultured with one or more cytokines.

In some embodiments, an engineered cell of the present disclosure can include, but is not limited to, a T cell (e.g., a CD8+ T cell, a CD4+ T cell, or a gamma-delta T cell), a cytotoxic T lymphocyte (CTL), a regulatory T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage (e.g., an M1 macrophage or an M2 macrophage), a monocyte, a dendritic cell, an erythrocyte, a platelet cell, a neuron, an oligodendrocyte, an astrocyte, a placode-derived cell, a Schwann cell, a cardiomyocyte, an endothelial cell, a nodal cell, a microglial cell, a hepatocyte, a cholangiocyte, a beta cell, a human embryonic stem cell (ESC), an ESC-derived cell, a pluripotent stem cell, a mesenchymal stromal cell (MSC), an induced pluripotent stem cell (iPSC), and an iPSC-derived cell.

In some embodiments, an engineered cell of the present disclosure is a T cell (e.g., a CD8+ T cell, a CD4+ T cell, or a gamma-delta T cell). In some embodiments, an engineered of the present disclosure is a cytotoxic T lymphocyte (CTL). In some embodiments, an engineered cell of the present disclosure is a regulatory T cell. In some embodiments, an engineered cell of the present disclosure is a Natural Killer T (NKT) cell. In some embodiments, an engineered cell of the present disclosure is a Natural Killer (NK) cell. In some embodiments, an engineered cell of the present disclosure is a B cell. In some embodiments, an engineered cell of the present disclosure is a tumor-infiltrating lymphocyte (TIL). In some embodiments, an engineered cell of the present disclosure is an innate lymphoid cell. In some embodiments, an engineered cell of the present disclosure is a mast cell. In some embodiments, an engineered cell of the present disclosure is an eosinophil. In some embodiments, an engineered cell of the present disclosure is a basophil. In some embodiments, an engineered cell of the present disclosure is a neutrophil. In some embodiments, an engineered cell of the present disclosure is a myeloid cell. In some embodiments, an engineered cell of the present disclosure is a macrophage e.g., an M1 macrophage or an M2 macrophage). In some embodiments, an engineered cell of the present disclosure is a monocyte. In some embodiments, an engineered or isolated cell of the present disclosure is a dendritic cell. In some embodiments, an engineered cell of the present disclosure is an erythrocyte. In some embodiments, an engineered cell of the present disclosure is a platelet cell. In some embodiments, a cell of the present disclosure is a neuron. In some embodiments, a cell of the present disclosure is a microglial cell. In some embodiments, a cell of the present disclosure is an oligodendrocyte. In some embodiments, a cell of the present disclosure is an astrocyte. In some embodiments, a cell of the present disclosure is a placode-derived cell. In some embodiments, an engineered cell of the present disclosure is a Schwann cell. In some embodiments, an engineered cell of the present disclosure is a cardiomyocyte. In some embodiments, an engineered cell of the present disclosure is an endothelial cell. In some embodiments, an engineered cell of the present disclosure is a nodal cell. In some embodiments, an engineered cell of the present disclosure is a microglial cell. In some embodiments, an engineered cell of the present disclosure is a hepatocyte. In some embodiments, an engineered cell of the present disclosure is a cholangiocyte. In some embodiments, an engineered cell of the present disclosure is a beta cell. In some embodiments, an engineered cell of the present disclosure is a human embryonic stem cell (ESC). In some embodiments, an engineered cell of the present disclosure is an ESC-derived cell. In some embodiments, an engineered cell of the present disclosure is a pluripotent stem cell. In some embodiments, an engineered cell of the present disclosure is a mesenchymal stromal cell (MSC). In some embodiments, an engineered cell of the present disclosure is an induced pluripotent stem cell (iPSC). In some embodiments, an engineered cell of the present disclosure is an iPSC-derived cell. In some embodiments, an engineered cell is autologous. In some embodiments, an engineered cell is allogeneic. In some embodiments, an engineered cell of the present disclosure is a CD34+ cell, a CD3+ cell, a CD8+ cell, a CD16+ cell, and/or a CD4+ cell.

In some embodiments, a cell of the present disclosure is a tumor cell selected from: an adenocarcinoma cell, a bladder tumor cell, a brain tumor cell (e.g., a glioma cell or a glioblastoma cell), a breast tumor cell, a cervical tumor cell, a colorectal tumor cell, an esophageal tumor cell, a glioma cell, a kidney tumor cell, a liver tumor cell, a lung tumor cell, a melanoma cell, a mesothelioma cell, an ovarian tumor cell, a pancreatic tumor cell, a prostate tumor cell, a skin tumor cell, a thyroid tumor cell, and a uterine tumor cell.

Also provided herein are methods that include culturing the engineered cells of the present disclosure. Methods of culturing the engineered cells described herein are known. One skilled in the art will recognize that culturing conditions will depend on the particular engineered cell of interest. One skilled in the art will recognize that culturing conditions will depend on the specific downstream use of the engineered cell, for example, specific culturing conditions for subsequent administration of the engineered cell to a subject.

Methods of Engineering Cells

Also provided herein are compositions and methods for engineering cells with any polynucleotide molecule or construct as described herein.

In general, cells are engineered through introduction (i.e., delivery) of one or more polynucleotides of the present disclosure. Delivery methods include, but are not limited to, viral-mediated delivery, lipid-mediated transfection, nanoparticle delivery, electroporation, sonication, and cell membrane deformation by physical means. One skilled in the art will appreciate the choice of delivery method can depend on the specific cell type to be engineered.

### Viral-Mediated Delivery

Viral vector-based delivery platforms can be used to engineer cells. In general, a viral vector-based delivery platform engineers a cell through introducing (i.e., delivering) into a host cell. For example, a viral vector-based delivery platform can engineer a cell through introducing any of the engineered nucleic acids described herein. A viral vector-based delivery platform can be a nucleic acid, and as such, an engineered nucleic acid can also encompass an engineered virally derived nucleic acid. Such engineered virally derived nucleic acids can also be referred to as recombinant viruses or engineered viruses.

A viral vector-based delivery platform can encode more than one engineered nucleic acid, gene, or transgene within the same nucleic acid. For example, an engineered virally derived nucleic acid, e.g., a recombinant virus or an engineered virus, can encode one or more transgenes, including, but not limited to, any of the engineered nucleic acids described herein that encode one or more effector molecules. The one or more transgenes encoding the one or more effector molecules can be configured to express the one or more effector molecules. A viral vector-based delivery platform can encode one or more genes in addition to the one or more transgenes (e.g., transgenes encoding the one or more effector molecules), such as viral genes needed for viral infectivity and/or viral production (e.g., capsid proteins, envelope proteins, viral polymerases, viral transcriptases, etc.), referred to as cis-acting elements or genes.

A viral vector-based delivery platform can comprise more than one viral vector, such as separate viral vectors encoding the engineered nucleic acids, genes, or transgenes described herein, and referred to as trans-acting elements or genes. For example, a helper-dependent viral vector-based delivery platform can provide additional genes needed for viral infectivity and/or viral production on one or more additional separate vectors in addition to the vector encoding the one or more effector molecules. One viral vector can deliver more than one engineered nucleic acids, such as one vector that delivers engineered nucleic acids that are configured to produce two or more effector molecules. More than one viral vector can deliver more than one engineered nucleic acids, such as more than one vector that delivers one or more engineered nucleic acid configured to produce one or more effector molecules. The number of viral vectors used can depend on the packaging capacity of the above-mentioned viral vector-based vaccine platforms, and one skilled in the art can select the appropriate number of viral vectors.

In general, any of the viral vector-based systems can be used for the in vitro production of molecules, such as effector molecules, or used in vivo and ex vivo gene therapy procedures, e.g., for in vivo delivery of the engineered nucleic acids encoding one or more effector molecules. The selection of an appropriate viral vector-based system will depend on a variety of factors, such as cargo/payload size, immunogenicity of the viral system, target cell of interest, gene expression strength and timing, and other factors appreciated by one skilled in the art.

Viral vector-based delivery platforms can be RNA-based viruses or DNA-based viruses. Exemplary viral vector-based delivery platforms include, but are not limited to, a herpes simplex virus, an adenovirus, a measles virus, an influenza virus, a Indiana vesiculovirus, a Newcastle disease virus, a vaccinia virus, a poliovirus, a myxoma virus, a reovirus, a mumps virus, a Maraba virus, a rabies virus, a rotavirus, a hepatitis virus, a rubella virus, a dengue virus, a chikungunya virus, a respiratory syncytial virus, a lymphocytic choriomeningitis virus, a morbillivirus, a lentivirus, a replicating retrovirus, a rhabdovirus, a Seneca Valley virus, a sindbis virus, and any variant or derivative thereof. Other exemplary viral vector-based delivery platforms are described in the art, such as vaccinia, fowlpox, self-replicating alphavirus, marabavirus, adenovirus (See, e.g., Tatsis et al., Adenoviruses, Molecular Therapy (2004) 10, 616-629), or lentivirus, including but not limited to second, third or hybrid second/third generation lentivirus and recombinant lentivirus of any generation designed to target specific cell types or receptors (See, e.g., Hu et al., Immunization Delivered by Lentiviral Vectors for Cancer and Infectious Diseases, Immunol Rev. (2011) 239(1): 45-61, Sakuma et al., Lentiviral vectors: basic to translational, Biochem J. (2012) 443(3):603-18, Cooper et al., Rescue of splicing-mediated intron loss maximizes expression in lentiviral vectors containing the human ubiquitin C promoter, Nucl. Acids Res. (2015) 43 (1): 682-690, Zufferey et al., Self-Inactivating Lentivirus Vector for Safe and Efficient In vivo Gene Delivery, J. Virol. (1998) 72 (12): 9873-9880).

The sequences may be preceded with one or more sequences targeting a subcellular compartment. Upon introduction (i.e., delivery) into a host cell, infected cells (i.e., an engineered cell) can express, and in some case secrete, the one or more effector molecules. Vaccinia vectors and methods useful in immunization protocols are described in, e.g., U.S. Pat. No. 4,722,848. Another vector is BCG (Bacille Calmette Guerin). BCG vectors are described in Stover et al. (Nature 351:456-460 (1991)). A wide variety of other vectors useful for the introduction (i.e., delivery) of engineered nucleic acids, e.g., Salmonella typhi vectors, and the like will be apparent to those skilled in the art from the description herein.

The viral vector-based delivery platforms can be a virus that targets a tumor cell, herein referred to as an oncolytic virus. Examples of oncolytic viruses include, but are not limited to, an oncolytic herpes simplex virus, an oncolytic adenovirus, an oncolytic measles virus, an oncolytic influenza virus, an oncolytic Indiana vesiculovirus, an oncolytic Newcastle disease virus, an oncolytic vaccinia virus, an oncolytic poliovirus, an oncolytic myxoma virus, an oncolytic reovirus, an oncolytic mumps virus, an oncolytic Maraba virus, an oncolytic rabies virus, an oncolytic rotavirus, an oncolytic hepatitis virus, an oncolytic rubella virus, an oncolytic dengue virus, an oncolytic chikungunya virus, an oncolytic respiratory syncytial virus, an oncolytic lymphocytic choriomeningitis virus, an oncolytic morbillivirus, an oncolytic lentivirus, an oncolytic replicating retrovirus, an oncolytic rhabdovirus, an oncolytic Seneca Valley virus, an oncolytic sindbis virus, and any variant or derivative thereof. Any of the oncolytic viruses described herein can be a recombinant oncolytic virus comprising one more transgenes (e.g., an engineered nucleic acid) encoding one or more effector molecules. The transgenes encoding the one or more effector molecules can be configured to express the one or more effector molecules.

In some embodiments, the virus can include, but is not limited to, a lentivirus, a retrovirus, an oncolytic virus, an adenovirus, an adeno-associated virus (AAV), and a virus-like particle (VLP).

The viral vector-based delivery platform can be retrovirus-based. In general, retroviral vectors are comprised of cis-acting long terminal repeats with packaging capacity for up to 6-10 kb of foreign sequence. The minimum cis-acting LTRs are sufficient for replication and packaging of the vectors, which are then used to integrate the one or more engineered nucleic acids (e.g., transgenes encoding the one or more effector molecules) into the target cell to provide permanent transgene expression. Retroviral-based delivery systems include, but are not limited to, those based upon murine leukemia, virus (MuLV), gibbon ape leukemia virus (GaLV), Simian Immunodeficiency virus (SIV), human immunodeficiency virus (HIV), and combinations thereof (see, e.g., Buchscher et al., J. Virol. 66:2731-2739 (1992); Johann et ah, J. Virol. 66:1635-1640 (1992); Sommnerfelt et al., Virol. 176:58-59 (1990); Wilson et ah, J. Virol. 63:2374-2378 (1989); Miller et al, J, Virol. 65:2220-2224 (1991); PCT/US94/05700). Other retroviral systems include the Phoenix retrovirus system.

The viral vector-based delivery platform can be lentivirus-based. In general, lentiviral vectors are retroviral vectors that are able to transduce or infect non-dividing cells and typically produce high viral titers. Lentiviral-based delivery platforms can be HIV-based, such as ViraPower systems (ThermoFisher) or pLenti systems (Cell Biolabs). Lentiviral-based delivery platforms can be SIV, or FIV-based. Other exemplary lentivirus-based delivery platforms are described in more detail in U.S. Pat. Nos. 7,311,907; 7,262,049; 7,250,299; 7,226,780; 7,220,578; 7,211,247; 7,160,721; 7,078,031; 7,070,993; 7,056,699; 6,955,919, each herein incorporated by reference for all purposes.

The viral vector-based delivery platform can be adenovirus-based. In general, adenoviral based vectors are capable of very high transduction efficiency in many cell types, do not require cell division, achieve high titer and levels of expression, and can be produced in large quantities in a relatively simple system. In general, adenoviruses can be used for transient expression of a transgene within an infected cell since adenoviruses do not typically integrate into a host's genome. Adenovirus-based delivery platforms are described in more detail in Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655, each herein incorporated by reference for all purposes. Other exemplary adenovirus-based delivery platforms are described in more detail in U.S. Pat. Nos. 5,585,362; 6,083,716, 7,371,570; 7,348,178; 7,323,177; 7,319,033; 7,318,919; and 7,306,793 and International Patent Application WO96/13597, each herein incorporated by reference for all purposes.

The viral vector-based delivery platform can be adeno-associated virus (AAV)-based. Adeno-associated virus (“AAV”) vectors may be used to transduce cells with engineered nucleic acids (e.g., any of the engineered nucleic acids described herein). AAV systems can be used for the in vitro production of effector molecules, or used in vivo and ex vivo gene therapy procedures, e.g., for in vivo delivery of the engineered nucleic acids encoding one or more effector molecules (see, e.g., West et al., Virology 160:38-47 (1987); U.S. Pat. Nos. 4,797,368; 5,436,146; 6,632,670; 6,642,051; 7,078,387; 7,314,912; 6,498,244; 7,906,111; US patent publications US 2003-0138772, US 2007/0036760, and US 2009/0197338; Gao, et al., J. Virol, 78(12):6381-6388 (June 2004); Gao, et al, Proc Natl Acad Sci USA, 100(10):6081-6086 (May 13, 2003); and International Patent applications WO 2010/138263 and WO 93/24641; Kotin, Human Gene Therapy 5:793-801 (1994); Muzyczka, J. Clin. Invest. 94:1351 (1994), each herein incorporated by reference for all purposes). Exemplary methods for constructing recombinant AAV vectors are described in more detail in U.S. Pat. No. 5,173,414; Tratschin et ah, Mol. Cell. Biol. 5:3251-3260 (1985); Tratschin, et ah, Mol. Cell, Biol. 4:2072-2081 (1984); Hermonat &amp; Muzyczka, PNAS 81:64666470 (1984); and Samuiski et ah, J. Virol. 63:03822-3828 (1989), each herein incorporated by reference for all purposes. In general, an AAV-based vector comprises a capsid protein having an amino acid sequence corresponding to any one of AAV1, AAV2, AAV3, AAV4, AAV5, AAV6, AAV7, AAV8, AAV9, AAV.Rh10, AAV11 and variants thereof.

The viral vector-based delivery platform can be a virus-like particle (VLP) platform. In general, VLPs are constructed by producing viral structural proteins and purifying resulting viral particles. Then, following purification, a cargo/payload (e.g., any of the engineered nucleic acids described herein) is encapsulated within the purified particle ex vivo. Accordingly, production of VLPs maintains separation of the nucleic acids encoding viral structural proteins and the nucleic acids encoding the cargo/payload. The viral structural proteins used in VLP production can be produced in a variety of expression systems, including mammalian, yeast, insect, bacterial, or in vivo translation expression systems. The purified viral particles can be denatured and reformed in the presence of the desired cargo to produce VLPs using methods known to those skilled in the art. Production of VLPs are described in more detail in Seow et al. (Mol Ther. 2009 May; 17(5): 767-777), herein incorporated by reference for all purposes.

The viral vector-based delivery platform can be engineered to target (i.e., infect) a range of cells, target a narrow subset of cells, or target a specific cell. In general, the envelope protein chosen for the viral vector-based delivery platform will determine the viral tropism. The virus used in the viral vector-based delivery platform can be pseudotyped to target a specific cell of interest. The viral vector-based delivery platform can be pantropic and infect a range of cells. For example, pantropic viral vector-based delivery platforms can include the VSV-G envelope. The viral vector-based delivery platform can be amphotropic and infect mammalian cells. Accordingly, one skilled in the art can select the appropriate tropism, pseudotype, and/or envelope protein for targeting a desired cell type.

### Lipid Structure Delivery Systems

Engineered nucleic acids of the present disclosure (e.g., a polynucleotide molecule encoding a chimeric polypeptide) can be introduced into a cell using a lipid-mediated delivery system. In general, a lipid-mediated delivery system uses a structure composed of an outer lipid membrane enveloping an internal compartment. Examples of lipid-based structures include, but are not limited to, a lipid-based nanoparticle, a liposome, a micelle, an exosome, a vesicle, an extracellular vesicle, a cell, or a tissue. Lipid structure delivery systems can deliver a cargo/payload (e.g., any of the engineered nucleic acids described herein) in vitro, in vivo, or ex vivo.

A lipid-based nanoparticle can include, but is not limited to, a unilamellar liposome, a multilamellar liposome, and a lipid preparation. As used herein, a “liposome” is a generic term encompassing in vitro preparations of lipid vehicles formed by enclosing a desired cargo, e.g., an engineered nucleic acid, such as any of the engineered nucleic acids described herein, within a lipid shell or a lipid aggregate. Liposomes may be characterized as having vesicular structures with a bilayer membrane, generally comprising a phospholipid, and an inner medium that generally comprises an aqueous composition. Liposomes include, but are not limited to, emulsions, foams, micelles, insoluble monolayers, liquid crystals, phospholipid dispersions, lamellar layers and the like. Liposomes can be unilamellar liposomes. Liposomes can be multilamellar liposomes. Liposomes can be multivesicular liposomes. Liposomes can be positively charged, negatively charged, or neutrally charged. In certain embodiments, the liposomes are neutral in charge. Liposomes can be formed from standard vesicle-forming lipids, which generally include neutral and negatively charged phospholipids and a sterol, such as cholesterol. The selection of lipids is generally guided by consideration of a desired purpose, e.g., criteria for in vivo delivery, such as liposome size, acid lability and stability of the liposomes in the blood stream. A variety of methods are available for preparing liposomes, as described in, e.g., Szoka et al., Ann. Rev. Biophys. Bioeng. 9; 467 (1980), U.S. Pat. Nos. 4,235,871, 4,501,728, 4,501,728, 4,837,028, and 5,019,369, each herein incorporated by reference for all purposes.

A multilamellar liposome is generated spontaneously when lipids comprising phospholipids are suspended in an excess of aqueous solution such that multiple lipid layers are separated by an aqueous medium. Water and dissolved solutes are entrapped in closed structures between the lipid bilayers following the lipid components undergoing self-rearrangement. A desired cargo (e.g., a polypeptide, a nucleic acid, a small molecule drug, an engineered nucleic acid, such as any of the engineered nucleic acids described herein, a viral vector, a viral-based delivery system, etc.) can be encapsulated in the aqueous interior of a liposome, attached to a liposome via a linking molecule that is associated with both the liposome and the polypeptide/nucleic acid, interspersed within the lipid bilayer of a liposome, entrapped in a liposome, complexed with a liposome, or otherwise associated with the liposome such that it can be delivered to a target entity. Lipophilic molecules or molecules with lipophilic regions may also dissolve in or associate with the lipid bilayer.

A liposome used according to the present embodiments can be made by different methods, as would be known to one of ordinary skill in the art. Preparations of liposomes are described in further detail in WO 2016/201323, International Applications PCT/US85/01161 and PCT/US89/05040, and U.S. Pat. Nos. 4,728,578, 4,728,575, 4,737,323, 4,533,254, 4,162,282, 4,310,505, and 4,921,706; each herein incorporated by reference for all purposes.

Liposomes can be cationic liposomes. Examples of cationic liposomes are described in more detail in U.S. Pat. Nos. 5,962,016; 5,030,453; 6,680,068, U.S. Application 2004/0208921, and International Patent Applications WO03/015757A1, WO04029213A2, and WO02/100435A1, each hereby incorporated by reference in their entirety.

Lipid-mediated gene delivery methods are described, for instance, in WO 96/18372; WO 93/24640; Mannino & Gould-Fogerite, BioTechniques 6(7): 682-691 (1988); U.S. Pat. No. 5,279,833 Rose U.S. Pat. No. 5,279,833; WO91/06309; and Feigner et al., Proc. Natl. Acad. Sci. USA 84: 7413-7414 (1987), each herein incorporated by reference for all purposes.

Exosomes are small membrane vesicles of endocytic origin that are released into the extracellular environment following fusion of multivesicular bodies with the plasma membrane. The size of exosomes ranges between 30 and 100 nm in diameter. Their surface consists of a lipid bilayer from the donor cell's cell membrane, and they contain cytosol from the cell that produced the exosome, and exhibit membrane proteins from the parental cell on the surface. Exosomes useful for the delivery of nucleic acids are known to those skilled in the art, e.g., the exosomes described in more detail in U.S. Pat. No. 9,889,210, herein incorporated by reference for all purposes.

As used herein, the term “extracellular vesicle” or “EV” refers to a cell-derived vesicle comprising a membrane that encloses an internal space. In general, extracellular vesicles comprise all membrane-bound vesicles that have a smaller diameter than the cell from which they are derived. Generally extracellular vesicles range in diameter from 20 nm to 1000 nm, and can comprise various macromolecular cargo either within the internal space, displayed on the external surface of the extracellular vesicle, and/or spanning the membrane. The cargo can comprise nucleic acids (e.g., any of the engineered nucleic acids described herein), proteins, carbohydrates, lipids, small molecules, and/or combinations thereof. By way of example and without limitation, extracellular vesicles include apoptotic bodies, fragments of cells, vesicles derived from cells by direct or indirect manipulation (e.g., by serial extrusion or treatment with alkaline solutions), vesiculated organelles, and vesicles produced by living cells (e.g., by direct plasma membrane budding or fusion of the late endosome with the plasma membrane). Extracellular vesicles can be derived from a living or dead organism, explanted tissues or organs, and/or cultured cells.

As used herein the term “exosome” refers to a cell-derived small (between 20-300 nm in diameter, more preferably 40-200 nm in diameter) vesicle comprising a membrane that encloses an internal space, and which is generated from the cell by direct plasma membrane budding or by fusion of the late endosome with the plasma membrane. The exosome comprises lipid or fatty acid and polypeptide and optionally comprises a payload (e.g., a therapeutic agent), a receiver (e.g., a targeting moiety), a polynucleotide (e.g., a nucleic acid, RNA, or DNA, such as any of the engineered nucleic acids described herein), a sugar (e.g., a simple sugar, polysaccharide, or glycan) or other molecules. The exosome can be derived from a producer cell, and isolated from the producer cell based on its size, density, biochemical parameters, or a combination thereof. An exosome is a species of extracellular vesicle. Generally, exosome production/biogenesis does not result in the destruction of the producer cell. Exosomes and preparation of exosomes are described in further detail in WO 2016/201323, which is hereby incorporated by reference in its entirety.

As used herein, the term “nanovesicle” (also referred to as a “microvesicle”) refers to a cell-derived small (between 20-250 nm in diameter, more preferably 30-150 nm in diameter) vesicle comprising a membrane that encloses an internal space, and which is generated from the cell by direct or indirect manipulation such that said nanovesicle would not be produced by said producer cell without said manipulation. In general, a nanovesicle is a sub-species of an extracellular vesicle. Appropriate manipulations of the producer cell include but are not limited to serial extrusion, treatment with alkaline solutions, sonication, or combinations thereof. The production of nanovesicles may, in some instances, result in the destruction of said producer cell. Preferably, populations of nanovesicles are substantially free of vesicles that are derived from producer cells by way of direct budding from the plasma membrane or fusion of the late endosome with the plasma membrane. The nanovesicle comprises lipid or fatty acid and polypeptide, and optionally comprises a payload (e.g., a therapeutic agent), a receiver (e.g., a targeting moiety), a polynucleotide (e.g., a nucleic acid, RNA, or DNA, such as any of the engineered nucleic acids described herein), a sugar (e.g., a simple sugar, polysaccharide, or glycan) or other molecules. The nanovesicle, once it is derived from a producer cell according to said manipulation, may be isolated from the producer cell based on its size, density, biochemical parameters, or a combination thereof.

Lipid nanoparticles (LNPs), in general, are synthetic lipid structures that rely on the amphiphilic nature of lipids to form membranes and vesicle like structures (Riley 2017). In general, these vesicles deliver cargo/payloads, such as any of the engineered nucleic acids or viral systems described herein, by absorbing into the membrane of target cells and releasing the cargo into the cytosol. Lipids used in LNP formation can be cationic, anionic, or neutral. The lipids can be synthetic or naturally derived, and in some instances biodegradable. Lipids can include fats, cholesterol, phospholipids, lipid conjugates including, but not limited to, polyethyleneglycol (PEG) conjugates (PEGylated lipids), waxes, oils, glycerides, and fat soluble vitamins. Lipid compositions generally include defined mixtures of materials, such as the cationic, neutral, anionic, and amphipathic lipids. In some instances, specific lipids are included to prevent LNP aggregation, prevent lipid oxidation, or provide functional chemical groups that facilitate attachment of additional moieties. Lipid composition can influence overall LNP size and stability. In an example, the lipid composition comprises dilinoleylmethyl-4-dimethylaminobutyrate (MC3) or MC3-like molecules. MC3 and MC3-like lipid compositions can be formulated to include one or more other lipids, such as a PEG or PEG-conjugated lipid, a sterol, or neutral lipids. In addition, LNPs can be further engineered or functionalized to facilitate targeting of specific cell types. Another consideration in LNP design is the balance between targeting efficiency and cytotoxicity.

Micelles, in general, are spherical synthetic lipid structures that are formed using single-chain lipids, where the single-chain lipid's hydrophilic head forms an outer layer or membrane and the single-chain lipid's hydrophobic tails form the micelle center. Micelles typically refer to lipid structures only containing a lipid mono-layer. Micelles are described in more detail in Quader et al. (Mol Ther. 2017 Jul. 5; 25(7): 1501-1513), herein incorporated by reference for all purposes.

Nucleic-acid vectors, such as expression vectors, exposed directly to serum can have several undesirable consequences, including degradation of the nucleic acid by serum nucleases or off-target stimulation of the immune system by the free nucleic acids. Similarly, viral delivery systems exposed directly to serum can trigger an undesired immune response and/or neutralization of the viral delivery system. Therefore, encapsulation of an engineered nucleic acid and/or viral delivery system can be used to avoid degradation, while also avoiding potential off-target affects. In certain examples, an engineered nucleic acid and/or viral delivery system is fully encapsulated within the delivery vehicle, such as within the aqueous interior of an LNP. Encapsulation of an engineered nucleic acid and/or viral delivery system within an LNP can be carried out by techniques well-known to those skilled in the art, such as microfluidic mixing and droplet generation carried out on a microfluidic droplet generating device. Such devices include, but are not limited to, standard T-junction devices or flow-focusing devices. In an example, the desired lipid formulation, such as MC3 or MC3-like containing compositions, is provided to the droplet generating device in parallel with an engineered nucleic acid or viral delivery system and any other desired agents, such that the delivery vector and desired agents are fully encapsulated within the interior of the MC3 or MC3-like based LNP. In an example, the droplet generating device can control the size range and size distribution of the LNPs produced. For example, the LNP can have a size ranging from 1 to 1000 nanometers in diameter, e.g., 1, 10, 50, 100, 500, or 1000 nanometers. Following droplet generation, the delivery vehicles encapsulating the cargo/payload (e.g., an engineered nucleic acid and/or viral delivery system) can be further treated or engineered to prepare them for administration.

### Nanoparticle Delivery

Nanomaterials can be used to deliver engineered nucleic acids (e.g., a polynucleotide molecule encoding a chimeric polypeptide). Nanomaterial vehicles, importantly, can be made of non-immunogenic materials and generally avoid eliciting immunity to the delivery vector itself. These materials can include, but are not limited to, lipids (as previously described), inorganic nanomaterials, and other polymeric materials. Nanomaterial particles are described in more detail in Riley et al. (Recent Advances in Nanomaterials for Gene Delivery—A Review. Nanomaterials 2017, 7(5), 94), herein incorporated by reference for all purposes.

### Genomic Editing Systems

Genomic editing systems can be used to engineer a host genome to encode an engineered nucleic acid, such as a polynucleotide molecule encoding a chimeric polypeptide of the present disclosure. In general, a “genomic editing system” refers to any system for integrating an exogenous gene into a host cell's genome. Genomic editing systems include, but are not limited to, a transposon system, a nuclease genomic editing system, and a viral vector-based delivery platform.

A transposon system can be used to integrate an engineered nucleic acid, such as an engineered nucleic acid of the present disclosure, into a host genome. Transposons generally comprise terminal inverted repeats (TIR) that flank a cargo/payload nucleic acid and a transposase. The transposon system can provide the transposon in cis or in trans with the TIR-flanked cargo. A transposon system can be a retrotransposon system or a DNA transposon system. In general, transposon systems integrate a cargo/payload (e.g., an engineered nucleic acid) randomly into a host genome. Examples of transposon systems include systems using a transposon of the Tcl/mariner transposon superfamily, such as a Sleeping Beauty transposon system, described in more detail in Hudecek et al. (Crit Rev Biochem Mol Biol. 2017 August; 52(4):355-380), and U.S. Pat. Nos. 6,489,458, 6,613,752 and 7,985,739, each of which is herein incorporated by reference for all purposes. Another example of a transposon system includes a PiggyBac transposon system, described in more detail in U.S. Pat. Nos. 6,218,185 and 6,962,810, each of which is herein incorporated by reference for all purposes.

A nuclease genomic editing system can be used to engineer a host genome to encode an engineered nucleic acid, such as an isolated polynucleotide or heterologous construct of the present disclosure. Without wishing to be bound by theory, in general, the nuclease-mediated gene editing systems used to introduce an exogenous gene take advantage of a cell's natural DNA repair mechanisms, particularly homologous recombination (HR) repair pathways. Briefly, following an insult to genomic DNA (typically a double-stranded break), a cell can resolve the insult by using another DNA source that has identical, or substantially identical, sequences at both its 5′ and 3′ ends as a template during DNA synthesis to repair the lesion. In a natural context, HDR can use the other chromosome present in a cell as a template. In gene editing systems, exogenous polynucleotides are introduced into the cell to be used as a homologous recombination template (HRT or HR template). In general, any additional exogenous sequence not originally found in the chromosome with the lesion that is included between the 5′ and 3′ complimentary ends within the HRT (e.g., a gene or a portion of a gene) can be incorporated (i.e., “integrated”) into the given genomic locus during templated HDR. Thus, a typical HR template for a given genomic locus has a nucleotide sequence identical to a first region of an endogenous genomic target locus, a nucleotide sequence identical to a second region of the endogenous genomic target locus, and a nucleotide sequence encoding a cargo/payload nucleic acid (e.g., any of the engineered nucleic acids described herein, such as any of the engineered nucleic acids encoding one or more effector molecules).

In some examples, a HR template can be linear. Examples of linear HR templates include, but are not limited to, a linearized plasmid vector, a ssDNA, a synthesized DNA, and a PCR amplified DNA. In particular examples, a HR template can be circular, such as a plasmid. A circular template can include a supercoiled template.

The identical, or substantially identical, sequences found at the 5′ and 3′ ends of the HR template, with respect to the exogenous sequence to be introduced, are generally referred to as arms (HR arms). HR arms can be identical to regions of the endogenous genomic target locus (i.e., 100% identical). HR arms in some examples can be substantially identical to regions of the endogenous genomic target locus. While substantially identical HR arms can be used, it can be advantageous for HR arms to be identical as the efficiency of the HDR pathway may be impacted by HR arms having less than 100% identity.

Each HR arm, i.e., the 5′ and 3′ HR arms, can be the same size or different sizes. Each HR arm can each be greater than or equal to 50, 100, 200, 300, 400, or 500 bases in length. Although HR arms can, in general, be of any length, practical considerations, such as the impact of HR arm length and overall template size on overall editing efficiency, can also be taken into account. An HR arms can be identical, or substantially identical to, regions of an endogenous genomic target locus immediately adjacent to a cleavage site. Each HR arms can be identical to, or substantially identical to, regions of an endogenous genomic target locus immediately adjacent to a cleavage site. Each HR arms can be identical, or substantially identical to, regions of an endogenous genomic target locus within a certain distance of a cleavage site, such as 1 base-pair, less than or equal to 10 base-pairs, less than or equal to 50 base-pairs, or less than or equal to 100 base-pairs of each other.

A nuclease genomic editing system can use a variety of nucleases to cut a target genomic locus, including, but not limited to, a Clustered Regularly Interspaced Short Palindromic Repeats (CRISPR) family nuclease or derivative thereof, a Transcription activator-like effector nuclease (TALEN) or derivative thereof, a zinc-finger nuclease (ZFN) or derivative thereof, and a homing endonuclease (HE) or derivative thereof.

A CRISPR-mediated gene editing system can be used to engineer a host genome to encode an engineered nucleic acid, such as an engineered nucleic acid encoding one or more of the effector molecules described herein. CRISPR systems are described in more detail in M. Adli (“The CRISPR tool kit for genome editing and beyond” Nature Communications; volume 9 (2018), Article number: 1911), herein incorporated by reference for all that it teaches. In general, a CRISPR-mediated gene editing system comprises a CRISPR-associated (Cas) nuclease and an RNA(s) that directs cleavage to a particular target sequence. An exemplary CRISPR-mediated gene editing system is the CRISPR/Cas9 systems comprised of a Cas9 nuclease and an RNA(s) that has a CRISPR RNA (crRNA) domain and a trans-activating CRISPR (tracrRNA) domain. The crRNA typically has two RNA domains: a guide RNA sequence (gRNA) that directs specificity through base-pair hybridization to a target sequence (“a defined nucleotide sequence”), e.g., a genomic sequence; and an RNA domain that hybridizes to a tracrRNA. A tracrRNA can interact with and thereby promote recruitment of a nuclease (e.g., Cas9) to a genomic locus. The crRNA and tracrRNA polynucleotides can be separate polynucleotides. The crRNA and tracrRNA polynucleotides can be a single polynucleotide, also referred to as a single guide RNA (sgRNA). While the Cas9 system is illustrated here, other CRISPR systems can be used, such as the Cpf1 system. Nucleases can include derivatives thereof, such as Cas9 functional mutants, e.g., a Cas9 “nickase” mutant that in general mediates cleavage of only a single strand of a defined nucleotide sequence as opposed to a complete double-stranded break typically produced by Cas9 enzymes.

In general, the components of a CRISPR system interact with each other to form a Ribonucleoprotein (RNP) complex to mediate sequence specific cleavage. In some CRISPR systems, each component can be separately produced and used to form the RNP complex. In some CRISPR systems, each component can be separately produced in vitro and contacted (i.e., “complexed”) with each other in vitro to form the RNP complex. The in vitro produced RNP can then be introduced (i.e., “delivered”) into a cell's cytosol and/or nucleus, e.g., a T cell's cytosol and/or nucleus. The in vitro produced RNP complexes can be delivered to a cell by a variety of means including, but not limited to, electroporation, lipid-mediated transfection, cell membrane deformation by physical means, lipid nanoparticles (LNP), virus like particles (VLP), and sonication. In a particular example, in vitro produced RNP complexes can be delivered to a cell using a Nucleofactor/Nucleofection® electroporation-based delivery system (Lonza®). Other electroporation systems include, but are not limited to, MaxCyte electroporation systems, Miltenyi CliniMACS electroporation systems, Neon electroporation systems, and BTX electroporation systems. CRISPR nucleases, e.g., Cas9, can be produced in vitro (i.e., synthesized and purified) using a variety of protein production techniques known to those skilled in the art. CRISPR system RNAs, e.g., an sgRNA, can be produced in vitro (i.e., synthesized and purified) using a variety of RNA production techniques known to those skilled in the art, such as in vitro transcription or chemical synthesis.

An in vitro produced RNP complex can be complexed at different ratios of nuclease to gRNA. An in vitro produced RNP complex can be also be used at different amounts in a CRISPR-mediated editing system. For example, depending on the number of cells desired to be edited, the total RNP amount added can be adjusted, such as a reduction in the amount of RNP complex added when editing a large number of cells in a reaction.

In some CRISPR systems, each component (e.g., Cas9 and an sgRNA) can be separately encoded by a polynucleotide with each polynucleotide introduced into a cell together or separately. In some CRISPR systems, each component can be encoded by a single polynucleotide (i.e., a multi-promoter or multicistronic vector, see description of exemplary multicistronic systems below) and introduced into a cell. Following expression of each polynucleotide encoded CRISPR component within a cell (e.g., translation of a nuclease and transcription of CRISPR RNAs), an RNP complex can form within the cell and can then direct site-specific cleavage.

Some RNPs can be engineered to have moieties that promote delivery of the RNP into the nucleus. For example, a Cas9 nuclease can have a nuclear localization signal (NLS) domain such that if a Cas9 RNP complex is delivered into a cell's cytosol or following translation of Cas9 and subsequent RNP formation, the NLS can promote further trafficking of a Cas9 RNP into the nucleus.

The cells described herein can be engineered using non-viral methods, e.g., the nuclease and/or CRISPR mediated gene editing systems described herein can be delivered to a cell using non-viral methods. The cells described herein can be engineered using viral methods, e.g., the nuclease and/or CRISPR mediated gene editing systems described herein can be delivered to a cell using viral methods such as adenoviral, retroviral, lentiviral, or any of the other viral-based delivery methods described herein.

In some CRISPR systems, more than one CRISPR composition can be provided such that each separately target the same gene or general genomic locus at more than target nucleotide sequence. For example, two separate CRISPR compositions can be provided to direct cleavage at two different target nucleotide sequences within a certain distance of each other. In some CRISPR systems, more than one CRISPR composition can be provided such that each separately target opposite strands of the same gene or general genomic locus. For example, two separate CRISPR “nickase” compositions can be provided to direct cleavage at the same gene or general genomic locus at opposite strands.

In general, the features of a CRISPR-mediated editing system described herein can apply to other nuclease-based genomic editing systems. TALEN is an engineered site-specific nuclease, which is composed of the DNA-binding domain of TALE (transcription activator-like effectors) and the catalytic domain of restriction endonuclease Fokl. By changing the amino acids present in the highly variable residue region of the monomers of the DNA binding domain, different artificial TALENs can be created to target various nucleotides sequences. The DNA binding domain subsequently directs the nuclease to the target sequences and creates a double-stranded break. TALEN-based systems are described in more detail in U.S. Ser. No. 12/965,590; U.S. Pat. Nos. 8,450,471; 8,440,431; U.S. Pat. Nos. 8,440,432; 10,172,880; and U.S. Ser. No. 13/738,381, all of which are incorporated by reference herein in their entirety. ZFN-based editing systems are described in more detail in U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; 2005/0267061, all incorporated herein by reference in their entireties for all purposes.

### Other Engineering Delivery Systems

Various additional means to introduce engineered nucleic acids (e.g., a polynucleotide molecule encoding a chimeric polypeptide as described herein) into a cell or other target recipient entity, such as any of the lipid structures described herein.

Electroporation can used to deliver polynucleotides to recipient entities. Electroporation is a method of internalizing a cargo/payload into a target cell or entity's interior compartment through applying an electrical field to transiently permeabilize the outer membrane or shell of the target cell or entity. In general, the method involves placing cells or target entities between two electrodes in a solution containing a cargo of interest (e.g., any of the engineered nucleic acids described herein). The lipid membrane of the cells is then disrupted, i.e., permeabilized, by applying a transient set voltage that allows the cargo to enter the interior of the entity, such as the cytoplasm of the cell. In the example of cells, at least some, if not a majority, of the cells remain viable. Cells and other entities can be electroporated in vitro, in vivo, or ex vivo. Electroporation conditions (e.g., number of cells, concentration of cargo, recovery conditions, voltage, time, capacitance, pulse type, pulse length, volume, cuvette length, electroporation solution composition, etc.) vary depending on several factors including, but not limited to, the type of cell or other recipient entity, the cargo to be delivered, the efficiency of internalization desired, and the viability desired. Optimization of such criteria are within the scope of those skilled in the art. A variety devices and protocols can be used for electroporation. Examples include, but are not limited to, Neon® Transfection System, MaxCyte® Flow Electroporation™, Lonza® Nucleofector™ systems, and Bio-Rad® electroporation systems.

Other means for introducing engineered nucleic acids (e.g., a polynucleotide molecule encoding a chimeric polypeptide as described herein) into a cell or other target recipient entity include, but are not limited to, sonication, gene gun, hydrodynamic injection, and cell membrane deformation by physical means.

Compositions and methods for delivering engineered mRNAs in vivo, such as naked plasmids or mRNA, are described in detail in Kowalski et al. (Mol Ther. 2019 Apr. 10; 27(4): 710-728) and Kaczmarek et al. (Genome Med. 2017; 9: 60.), each herein incorporated by reference for all purposes.

Methods of Use

Methods of using chimeric polypeptides, polynucleotide molecules, or cells as described herein are also encompassed by this disclosure.

In some embodiments, the methods include inhibiting repression of a gene of interest. Methods of inhibiting repression may include: providing a transformed cell comprising an expression system comprising (i) an expression cassette encoding the chimeric polypeptide as described herein, and (ii) a target expression cassette comprising an ITM-responsive promoter operably linked to a gene of interest; culturing the transformed cell under conditions suitable for expression of the chimeric polypeptide; and inducing degradation of the chimeric polypeptide by contacting the transformed cell with a ligand that promotes degradation of the chimeric polypeptide.

In some embodiments, inhibiting repression is measurable as at least a 1.5-fold increase, at least a 2-fold increase, at least a 3-fold increase, at least a 4-fold increase, or at least a 5-fold increase in expression of a gene of interest operably linked to an ITM-responsive promoter in a transformed cell, following contacting the transformed cell with the ligand, as compared to expression of the gene of interest in an equivalent transformed cell that was not contacted with the ligand.

In some embodiments, in the absence of the ligand, the expression level of the gene of interest is repressed by the ITM by at least 1.5-fold, at least 2-fold, at least 3-fold, at least 4-fold, or at least 5-fold, at least 6-fold, at least 7-fold, at least 8-fold, or at least 10-fold as compared to an expression level in the absence of the ITM.

### In Vivo Methods

The methods provided herein also include inhibiting transcriptional repression in vivo, e.g., by delivering a ligand that induces degradation of the chimeric polypeptide in vivo.

In some embodiments, the transformed cell is in a human or animal, and contacting the transformed cell with the ligand comprises administering a pharmacological dose of the ligand to the human or animal. In some embodiments, the ligand administered to the subject an immunomodulatory drug (IMiD) that promotes ubiquitin pathway-mediated degradation of the chimeric polypeptide. In some embodiments, the IMiD can include, but is not limited to, thalidomide, lenalidomide, and pomalidomide. In some embodiments, the IMiD is pomalidomide and is administered to the subject at a concentration of between about 1 mg per day and about 50 mg per day. In particular embodiments, the non-endogenous ligand is administered to the subject at a concentration of about 4 mg per day.

### Pharmaceutical Compositions

The chimeric polypeptides, isolated polynucleotides, and cells of the present disclosure can be formulated in pharmaceutical compositions. These compositions can comprise, in addition to one or more of the engineered nucleic acids or engineered cells, a pharmaceutically acceptable excipient, carrier, buffer, stabilizer or other materials well known to those skilled in the art. Such materials should be non-toxic and should not interfere with the efficacy of the active ingredient. The precise nature of the carrier or other material can depend on the route of administration, e.g. oral, intravenous, cutaneous or subcutaneous, nasal, intramuscular, intraperitoneal routes.

Whether it is a cell, polypeptide, nucleic acid, small molecule or other pharmaceutically useful compound according to the present disclosure that is to be given to an individual, administration is preferably in a “therapeutically effective amount” or “prophylactically effective amount” (as the case can be, although prophylaxis can be considered therapy), this being sufficient to show benefit to the individual. The actual amount administered, and rate and time-course of administration, will depend on the nature and severity of protein aggregation disease being treated. Prescription of treatment, e.g. decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

A composition can be administered alone or in combination with other treatments, either simultaneously or sequentially dependent upon the condition to be treated.

### Additional Embodiments

The paragraphs below provide additional enumerated embodiments:

Embodiment 1: A chimeric polypeptide comprising:
(a) an inducible transcription modulator (ITM), wherein the ITM comprises a transcriptional repressor domain and a DNA binding domain; and
(b) a degron,
wherein the degron is operably linked to the ITM, and
wherein the transcriptional repressor domain is selected from the group consisting of: a KRAB repression domain, an HDAC4 domain, a SCX HLH domain, a ID1 HLH domain, a HERC2 Cyt-b5 domain, a TWST1 HLH domain, an NKX22 homeodomain, an ID3 HLH domain, and a TWST2 HLH domain.

Embodiment 2: The chimeric polypeptide of embodiment 1, wherein the transcriptional repressor domain comprises the KRAB repression domain.

Embodiment 3: The chimeric polypeptide of embodiment 2, wherein the KRAB repression domain comprises minKRAB.

Embodiment 4: The chimeric polypeptide of any one of embodiments 1-3, wherein the KRAB repression domain comprises the amino acid sequence of SEQ ID NO: 2.

Embodiment 5: The chimeric polypeptide of embodiment 4, wherein the KRAB repression domain comprises a KRAB repressor domain variant of SEQ ID NO: 2 and comprises one or more amino acid substitutions selected from the group consisting of: W27L, K28L, D31A, T32A, Q34A, Q35A, R39E, L43S, T57C, K58C, P59C, V61Y, I62Y, I62A, L63W, L63Y, L63E, R64F, R64W, R64E, L65F, L65E, L65W, E66V, K67F, G68F, and E69F.

Embodiment 6: The chimeric polypeptide of embodiment 4, wherein the KRAB repression domain comprises a KRAB repressor domain variant of SEQ ID NO: 2 and comprises one or more amino acid substitutions selected from: Q34A/Q35A, I62A, T57C/K58C/P59C, D31A/T32A, L63W/R64W/L65W, E66V, L63E/R64E/L65E, R64F/L65F, W27L/K28L KRAB, R39E, K67F/G68F/E69F, and V61Y/I62Y/L63Y.

Embodiment 7: The chimeric polypeptide of any one of embodiments 1-3, wherein the KRAB repression domain comprises the amino acid sequence of SEQ ID NO: 3.

Embodiment 8: The chimeric polypeptide of embodiment 1, wherein the transcriptional repressor domain comprises the HDAC4 repression domain.

Embodiment 9: The chimeric polypeptide of embodiment 8, wherein the HDAC4 repression domain comprises the amino acid sequence of SEQ ID NO: 4.

Embodiment 10: The chimeric polypeptide of any one of embodiments 1-9, wherein the DNA binding domain comprises a zinc finger (ZF) protein domain.

Embodiment 11: The chimeric polypeptide of embodiment 10, wherein the ZF protein domain is modular in design and is composed of a zinc finger array (ZFA) of zinc finger motifs.

Embodiment 12: The chimeric polypeptide of embodiment 11, wherein the ZF protein domain comprises one to ten zinc finger motifs.

Embodiment 13: The chimeric polypeptide of embodiment 11, wherein the ZF protein domain comprises six zinc finger motifs.

Embodiment 14: The chimeric polypeptide of embodiment 13, wherein the ZF protein domain comprises SEQ ID NO: 5.

Embodiment 15: The chimeric polypeptide of any one of embodiments 1-14, wherein the transcriptional repressor domain is N-terminal to the DNA binding domain.

Embodiment 16: The chimeric polypeptide of any one of embodiments 1-14, wherein the transcriptional repressor domain is C-terminal to the DNA binding domain.

Embodiment 17: The chimeric polypeptide of any one of embodiments 1-16, wherein the transcriptional repressor domain and the DNA binding domain are separated by a first peptide linker.

Embodiment 18: The chimeric polypeptide of embodiment 17, wherein the first peptide linker comprises the amino acid sequence of GGGGSGGT (SEQ ID NO: 60).

Embodiment 19: The chimeric polypeptide of any one of embodiments 1-18, wherein the ITM is a synthetic transcription modulator.

Embodiment 20: The chimeric polypeptide of any one of embodiments 1-19 wherein the degron is selected from the group consisting of: HCV NS4 degron, PEST (two copies of residues 277-307 of human IκBα), GRR (residues 352-408 of human p105), DRR (residues 210-295 of yeast Cdc34), SNS (tandem repeat of SP2 and NB (SP2-NB-SP2 of influenza A or influenza B), RPB (four copies of residues 1688-1702 of yeast RPB), SPmix (tandem repeat of SP1 and SP2 (SP2-SP1-SP2-SP1-SP2 of influenza A virus M2 protein), NS2 (three copies of residues 79-93 of influenza A virus NS protein), ODC (residues 106-142 of ornithine decarboxylase), Nek2A, mouse ODC (residues 422-461), mouse ODC_DA (residues 422-461 of mODC including D433A and D434A point mutations), an APC/C degron, a COP1 E3 ligase binding degron motif, a CRL4-Cdt2 binding PIP degron, an actinfilin-binding degron, a KEAP1 binding degron, a KLHL2 and KLHL3 binding degron, an MDM2 binding motif, an N-degron, a hydroxyproline modification in hypoxia signaling, a phytohormone-dependent SCF-LRR-binding degron, an SCF ubiquitin ligase binding phosphodegron, a phytohormone-dependent SCF-LRR-binding degron, a DSGxxS phospho-dependent degron (“DSGxxS” disclosed as SEQ ID NO: 68), an Siah binding motif, an SPOP SBC docking motif, and a PCNA binding PIP box.

Embodiment 21: The chimeric polypeptide any one of embodiments 1-20, wherein the degron comprises a cereblon (CRBN) polypeptide substrate domain capable of binding CRBN in response to an immunomodulatory drug (IMiD).

Embodiment 22: The chimeric polypeptide of embodiment 21, wherein the CRBN polypeptide substrate domain is selected from the group consisting of: IKZF1, IKZF3, CKla, ZFP91, GSPT1, MEIS2, GSS E4F1, ZN276, ZN517, ZN582, ZN653, ZN654, ZN692, ZN787, and ZN827, or a fragment thereof that is capable of drug-inducible binding of CRBN.

Embodiment 23: The chimeric polypeptide of embodiment 21 or embodiment 22, wherein the CRBN polypeptide substrate domain comprises a chimeric fusion product of native CRBN polypeptide sequences.

Embodiment 24: The chimeric polypeptide of embodiment 21, wherein the CRBN polypeptide substrate domain comprises a IKZF3/ZFP91/IKZF3 chimeric fusion product having the amino acid sequence of

(SEQ ID NO: 6)
FNVLMVHKRSHTGERPLQCEICGFTCRQKGNLLRHIKLHTGEKPF
KCHLCNYACQRRDAL.

Embodiment 25: The chimeric polypeptide of any one of embodiments 21-24, wherein the IMiD is an FDA-approved drug.

Embodiment 26: The chimeric polypeptide of any one of embodiments 21-25, wherein the IMiD is selected from the group consisting of: thalidomide, lenalidomide, and pomalidomide.

Embodiment 27: The chimeric polypeptide of any one of embodiments 1-26, wherein the ITM is N-terminal to the degron.

Embodiment 28: The chimeric polypeptide of any one of embodiments 1-26, wherein the ITM is C-terminal to the degron.

Embodiment 29: The chimeric polypeptide of any one of embodiments 1-29, wherein the ITM is separated from the degron by a second peptide linker.

Embodiment 30: The chimeric polypeptide of embodiment 29, wherein the second peptide linker comprises an amino acid sequence selected from the group consisting of:

(SEQ ID NO: 7)
GSGSGSGS,
(SEQ ID NO: 8)
KEGS,
(SEQ ID NO: 9)
EGK, EAAAK,
and
(SEQ ID NO: 10)
AAPAKQE

Embodiment 31: The chimeric polypeptide of embodiment 29, wherein the second peptide linker comprises an amino acid sequence selected from the group consisting of:

(SEQ ID NO: 12)
AAPAKQEAAAPAKQEAAAPAKQ
EAAAPAPAAKAEAPAAAPAAKA
and
(SEQ ID NO: 13)
AEAAAKEAAAKEAAAKA

Embodiment 32: An expression cassette comprising a promoter operably linked to a polynucleotide sequence encoding the chimeric polypeptide of any one of embodiments 1-31.

Embodiment 33: The expression cassette of embodiment 32, wherein the promoter comprises a constitutive promoter.

Embodiment 34: The expression cassette of embodiment 33, wherein the constitutive promoter is selected from the group consisting of: CMV, EFS, SFFV, SV40, MND, PGK, UbC, hEF1a, hCAGG, hACTb, heIF4A1, hGAPDH, hGRP78, hGRP94, hHSP70, hKINb, and hUBIb.

Embodiment 35: The expression cassette of embodiment 32, wherein the promoter comprises an inducible promoter.

Embodiment 36: The expression cassette of embodiment 35, wherein the inducible promoter comprises a minimal promoter and a response element selected from the group consisting of: NFkB response element, CREB response element, NFAT response element, SRF response element 1, SRF response element 2, AP1 response element, TCF-LEF response element promoter fusion, Hypoxia responsive element, SMAD binding element, STAT3 binding site, inducer molecule responsive promoters, and tandem repeats thereof.

Embodiment 37: The expression cassette of embodiment 36, wherein the promoter comprises a synthetic promoter.

Embodiment 38: The expression cassette of any one of embodiments 32-37, wherein the polynucleotide sequence encoding the chimeric polypeptide further encodes a 3′untranslated region (UTR) comprising an mRNA-destabilizing element.

Embodiment 39: The expression cassette of embodiment 38, wherein the mRNA-destabilizing element is selected from the group consisting of: an AU-rich element and a stem-loop destabilizing element.

Embodiment 40: An expression system comprising the expression cassette of any of embodiments 32-39, and a target expression cassette comprising an ITM-responsive promoter operably linked to a gene of interest.

Embodiment 41: The expression system of embodiment 40, wherein the ITM-responsive promoter comprises a promoter sequence and a sequence that binds to the DNA binding domain of the ITM.

Embodiment 42: The expression system of embodiment 41, wherein the sequence that binds to the DNA binding domain comprises one or more zinc finger binding sites.

Embodiment 43: The expression system of embodiment 42, wherein the sequence that binds to the DNA binding domain comprises one or more zinc finger binding sites having the amino acid sequence of SEQ ID NO: 54.

Embodiment 44: The expression system of embodiment 42 or embodiment 43, wherein the sequence that binds to the DNA binding domain comprises four of more zinc finger binding sites.

Embodiment 45: The expression system of embodiment 44, wherein the sequence that binds to the DNA binding domain comprises the amino acid sequence of SEQ ID NO: 55.

Embodiment 46: The expression system of any one of embodiments 40-45, wherein the promoter sequence of the ITM-responsive promoter comprises a constitutive promoter sequence.

Embodiment 47: The expression system of embodiment 46, wherein the constitutive promoter sequence is selected from the group consisting of: CMV, EFS, SFFV, SV40, MND, PGK, UbC, EF1a, hCAGG, hACTb, heIF4A1, hGAPDH, hGRP78, hGRP94, hHSP70, hKINb, and hUBIb.

Embodiment 48: The expression system of any one of embodiments 40-47, wherein the promoter sequence of the ITM-responsive promoter comprises a minimal promoter.

Embodiment 49: The expression system of any one of embodiments 40-48, wherein the ITM-responsive promoter comprises a synthetic promoter.

Embodiment 50: The expression system of any one of embodiments 40-49, wherein the gene of interest encodes a therapeutic polypeptide.

Embodiment 51: The expression system of any one of embodiments 40-50, wherein the gene of interest encodes a polypeptide selected from the group consisting of: a cytokine, a chemokine, a homing molecule, a growth factor, a cell death regulator, a co-activation molecule, a tumor microenvironment modifier a, a receptor, a ligand, an antibody, a polynucleotide, a peptide, and an enzyme.

Embodiment 52: The expression system of any one of embodiments 40-51, comprising a heterologous construct comprising both of: (i) the expression cassette of any of embodiments 28-35 and (ii) the target expression cassette.

Embodiment 53: The expression system of any one of embodiments 40-51, comprising a first heterologous construct comprising the expression cassette of any of embodiments 32-39 and a second heterologous construct comprising the target expression cassette.

Embodiment 54: An isolated cell comprising the expression cassette of any one of embodiments 32-39.

Embodiment 55: An isolated cell comprising the expression system of any one of embodiments 40-53.

Embodiment 56: The isolated cell of embodiment 54 or 55, wherein the cell is a human cell.

Embodiment 57: The isolated cell of any one of embodiments 54-56, wherein the cell is a stem cell.

Embodiment 58: The isolated cell of any one of embodiments 54-56, wherein the cell is an immune cell.

Embodiment 59: The isolated cell of any one of embodiments 54-56, wherein the cell is selected from the group consisting of: a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an erythrocyte, a platelet cell, a human embryonic stem cell (ESC), an ESC-derived cell, a pluripotent stem cell, a mesenchymal stromal cell (MSC), an induced pluripotent stem cell (iPSC), and an iPSC-derived cell.

Embodiment 60: A genetic switch for inhibiting repression of a gene of interest, comprising: the chimeric polypeptide of any one of embodiments 1-31 and a ligand, wherein binding of the ligand to the degron induces degradation of the chimeric polypeptide, thereby inhibiting repression of the gene of interest, wherein the gene of interest is operably linked to an ITM-responsive promoter.

Embodiment 61: The genetic switch of embodiment 60, wherein the ligand comprises an immunomodulatory drug (IMiD) that promotes ubiquitin pathway-mediated degradation of the chimeric polypeptide.

Embodiment 62: The genetic switch of embodiment 61, wherein the IMiD is an FDA-approved drug.

Embodiment 63: The genetic switch of embodiment 61 or 62, wherein the IMiD is selected from the group consisting of: thalidomide, lenalidomide, and pomalidomide.

Embodiment 64: A method of inhibiting repression of a gene of interest, comprising:
(a) providing a cell comprising an expression system comprising (i) an expression cassette encoding the chimeric polypeptide of any one of embodiments 1-31, and (ii) a target expression cassette comprising an ITM-responsive promoter operably linked to a gene of interest; and
(b) inducing degradation of the chimeric polypeptide by contacting the transformed cell with a ligand that promotes degradation of the chimeric polypeptide.

Embodiment 65: The method of embodiment 64, wherein the method further comprises culturing the cell under conditions suitable for expression of the chimeric polypeptide.

Embodiment 66: The method of embodiment 64 or embodiment 65, wherein the expression cassette encoding the chimeric polypeptide comprises the expression cassette of any one of embodiments 32-39.

Embodiment 67: The method of any one of embodiments 64-66, wherein the expression system comprises the expression system of any one of embodiments 40-53.

Embodiment 68: A method of producing a cell that is capable of drug-regulated transcriptional repression, the method comprising transforming the cell with an expression cassette of any one of embodiments 32-39 or an expression system of any one of embodiments 40-56.

### Examples

Below are examples of specific embodiments for carrying out the present disclosure. The examples are offered for illustrative purposes only, and are not intended to limit the scope of the present disclosure in any way. Efforts have been made to ensure accuracy with respect to numbers used (e.g., amounts, temperatures, etc.), but some experimental error and deviation should, of course, be allowed for.

The practice of the present disclosure will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B(1992).

### Example 1: Efficient Degradation of Degron-Linked DNA Binders

Efficiency of degradation of degron-linked DNA binders following drug treatment was assessed.

### Materials and Methods

U87-MG cells were transduced (50,000 cells/transduction) with lentivirus (50,000 pg each, as titered by p24 assay) encoding each construct as shown in Table 4. Each construct format is described from 5′ to 3′ (ORF from N to C terminus) with sequences described in Table 4.

TABLE 4
Constructs tested for degradation efficiency

| Construct | |
|---|---|
| Name | Description |
| SB03433 | ZF10-BFP-A(EAAAK)3A-degron |
| SB03434 | ZF10-BFP-(GS)4GG-degron |
| SB03435 | ZF10-BFP-ecpd linker- degron |
| SB03436 | degron-A(EAAAK)3A-BFP-ZF10 |
| SB03437 | degron-(GS)4GG-BFP-ZF10 |
| SB03438 | ZF10-BFP-KEGS-degron |
| SB03439 | ZF10-BFP-EGK-degron |
| SB03440 | ZF10-BFP-ConMJ-degron |
| SB03441 | degron-KEGS-BFP-ZF10 |
| SB03442 | degron-EGK-BFP-ZF10 |
| SB03443 | degron-ConMJ-BFP-ZF10 |
| SB03444 | degron-(GS)4GG-BFP-ZF10-A(EAAAK)3A-degron |
| SB03445 | degron-A(EAAAK)3A-degron-(GS)4GG-BFP-ZF10 |
| SB02863 | degron-A(EAAAK)3A-ZF10-BFP |
| SB02861 | ZF10-BFP (negative ctrl) |

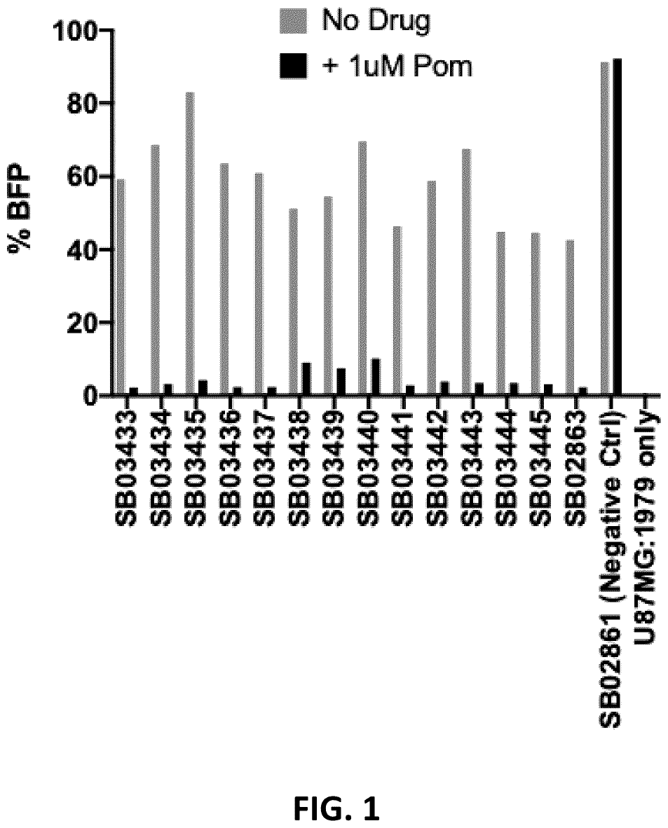

On day 2 following transduction, each cell group was cultured for 7 days in the presence 1 μM pomalidomide or in the absence of the drug, and blue fluorescent protein (BFP) expression was assessed by Fluorescence-Activated Cell Sorting (FACS). BFP expression for each cell group (with or without drug) is shown in FIG. 1.

### Results

Ubiquitin pathway-mediated degradation in response to immunomodulatory drug (IMiD) pomalidomide was assessed for constructs including a cereblon (CRBN) polypeptide substrate domain (referred to in Table 4 as “degron”) in various orientations and using various linkers. As shown in FIG. 1, for each construct tested, at least 40% of cells were BFP positive in the absence of drug, whereas 10% or fewer cells were BFP positive in the presence of pomalidomide. For each construct, the percentage of BFP positive cells in the presence of drug was compared to the percentage of BFP positive cells in the absence of drug to determine the percent degradation. Table 5 shows the percent degradation for each of the tested constructs. The results demonstrate IMiD-dependent degradation of constructs.

TABLE 5
Degradation efficiency following drug treatment

| Construct | |
|---|---|
| Name | % Degradation |
| SB03433 | 96.1 |
| SB03434 | 95.2 |
| SB03435 | 94.8 |
| SB03436 | 96.2 |
| SB03437 | 96.0 |
| SB03438 | 82.3 |
| SB03439 | 86.0 |
| SB03440 | 85.4 |
| SB03441 | 93.8 |
| SB03442 | 93.3 |
| SB03443 | 94.8 |
| SB03444 | 92.1 |
| SB03445 | 92.7 |
| SB02863 | 94.5 |
| SB02861 | −0.98 |

### Example 2: Degradation of Degron-Linked Repressors to Induce Transcription

A reporter cell line was produced by transducing U87-MG cells with lentivirus encoding mCherry linked to an ITM-responsive promoter of SEQ ID NO: 56 including a EF1a core promoter sequence with a zinc finger binding domain composed of four ZF10-1 binding sites upstream of the core promoter sequence.

The ability of degron-linked repressors to modulate protein expression of a reporter was assessed by transduction assay.

### Materials and Methods

A reporter cell line was produced by transducing U87-MG cells with lentivirus encoding mCherry linked to an ITM-responsive promoter of SEQ ID NO: 56 including an EF1a core promoter sequence with a zinc finger binding domain composed of four ZF10-1 binding sites upstream of the core promoter sequence. Reporter cells were transduced (50,000 cells/transduction) with lentivirus (50,000 pg each, as titered by p24 assay) encoding each construct as shown in Table 6. Each construct format is described from 5′ to 3′ (ORF from N to C terminus). Constructs SB03757 and SB03760 include a minKrab repression domain that is codon optimized for E. coli, and constructs SB03758 and SB03761. For constructs SB03758 and SB03761, the entire construct was codon optimized for E. coli.

TABLE 6
Constructs used for transducing reporter cells

| Construct | |
|---|---|
| Name | Description |
| SB03747 | degron-A(EAAAK)3A-ZF10-BFP-AuSLDE |
| SB03750 | BFP-ZF10-ecpd-degron-AuSLDE |
| SB03757 | degron-A(EAAAK)3A-ZF10-minKrab.ecoli.cod.opt |
| SB03758 | all .ecoli.cod.opt: degron-A(EAAAK)3A-ZF10-minKrab |
| SB03759 | degron-A(EAAAK)3A-ZF10-minKrab-AuSLDE |
| SB03760 | ecoli.cod.optminKrab-ZF10-ecpd-degron |
| SB03761 | all ecoli.cod.opt: minKrab-ZF10-ecpd-degron |
| SB03762 | minKrab-ZF10-ecpd-degron-AuSLDE |
| SB03255 | ZF10-minKrab-A(EAAAK)3A linker-degron |
| SB02131 | degron-A(EAAAK)3A-ZF10-minKrab |

On day 2 following transduction, cells were incubated either in the presence of 1 μM pomalidomide or in the absence of the drug. On day 5 of drug treatment, cells were assayed by Fluorescence-Activated Cell Sorting (FACS) for mCherry expression.

### Results

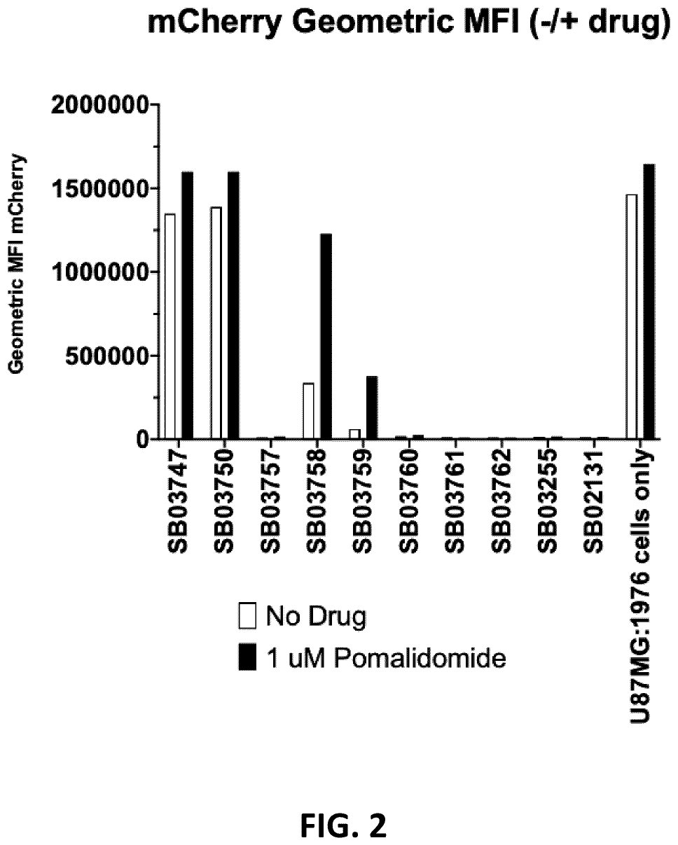

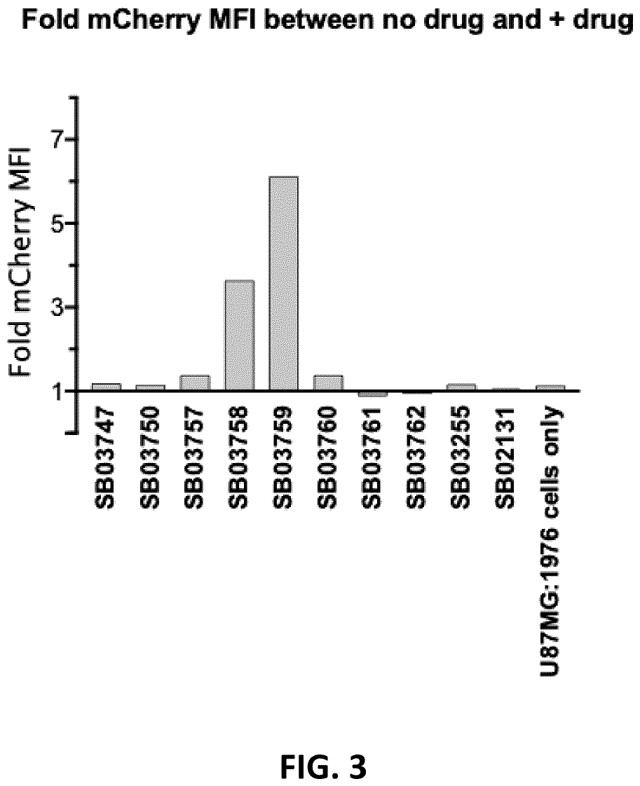

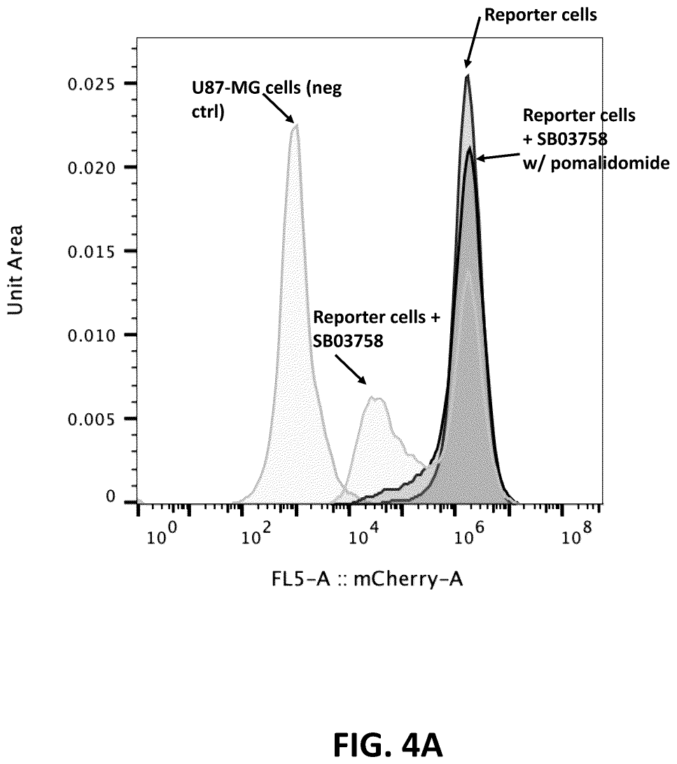

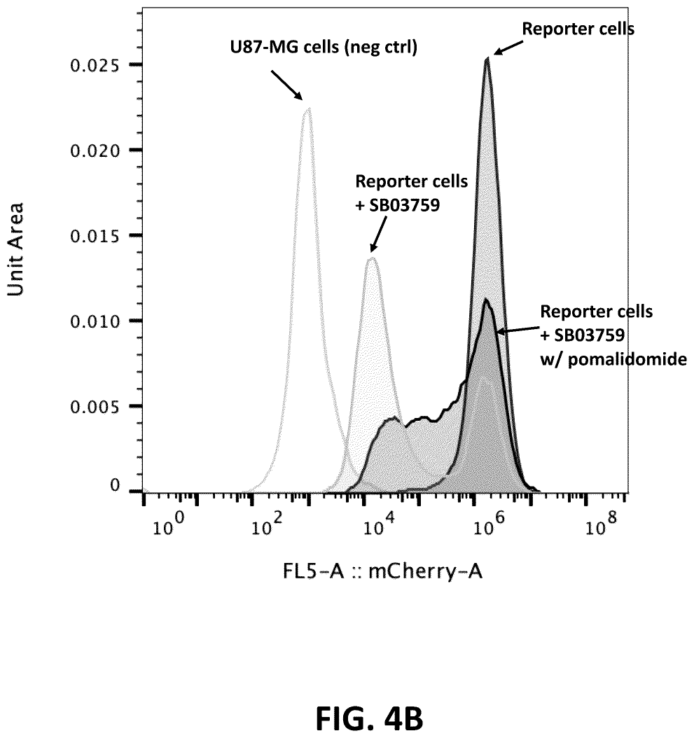

Degron-linked promoter regulation was assessed for constructs including a cereblon (CRBN) polypeptide substrate domain (d913) in various orientations and using various Krab-derived repression domains, linkers, and mRNA-destabilizing elements. The geometric mean fluorescent intensity (MFI) of mCherry for each condition is shown in FIG. 2. The fold difference between the geometric MFI of mCherry for the no drug condition versus the pomalidomide treatment condition was calculated as shown in FIG. 3. As shown in FIG. 3, drug-induced release of repression was possible with constructs SB03758 and SB03759 in response to pomalidomide treatment. Histogram plots of the mCherry expression for SB03758-transduced cells, in the presence or absence of pomalidomide, and as compared to untransduced cells (lacking the reporter), and reporter cells not transduced with a repressor, are shown in FIG. 4A. Histogram plots of the mCherry expression for SB03759-transduced cells, in the presence or absence of pomalidomide, and as compared to untransduced cells (lacking the reporter), and reporter cells not transduced with a repressor, are shown in FIG. 4B. The histogram plots shown in FIGS. 4A & 4B demonstrate that repression by SB03758 and SB03759 is regulatable in a drug-dependent manner

### Example 3: Optimization of Degradation of Degron-Linked Repressors

Constructs engineered to optimize drug-induced release of repression were assayed to directly test degradation efficiency.

### Materials and Methods

Reporter cells as described in Example 2 were transduced (50,000 cells/transduction) with lentivirus (50,000 pg each, as titered by p24 assay) encoding each construct as shown in Table 7, or were untransduced as a negative control. Each construct format is described from 5′ to 3′ (ORF from N to C terminus). Both SB03747 and SB03750 include an mRNA destabilization tag, AuSLDE, at the 3′ end. Seven days after transduction, and 5 days of treatment in the presence 1 μM pomalidomide or in the absence of the drug, BFP was measured by Fluorescence-Activated Cell Sorting (FACS) analysis.

TABLE 7
Constructs optimized for reversibility of repression

| Construct | Description |
|---|---|
| SB03747 | degron-A(EAAAK)3A-ZF10-1-BFP-AuSLDE |
| SB03750 | BFP-ZF10-1-ecpd-degron-AuSLDE |

### Results

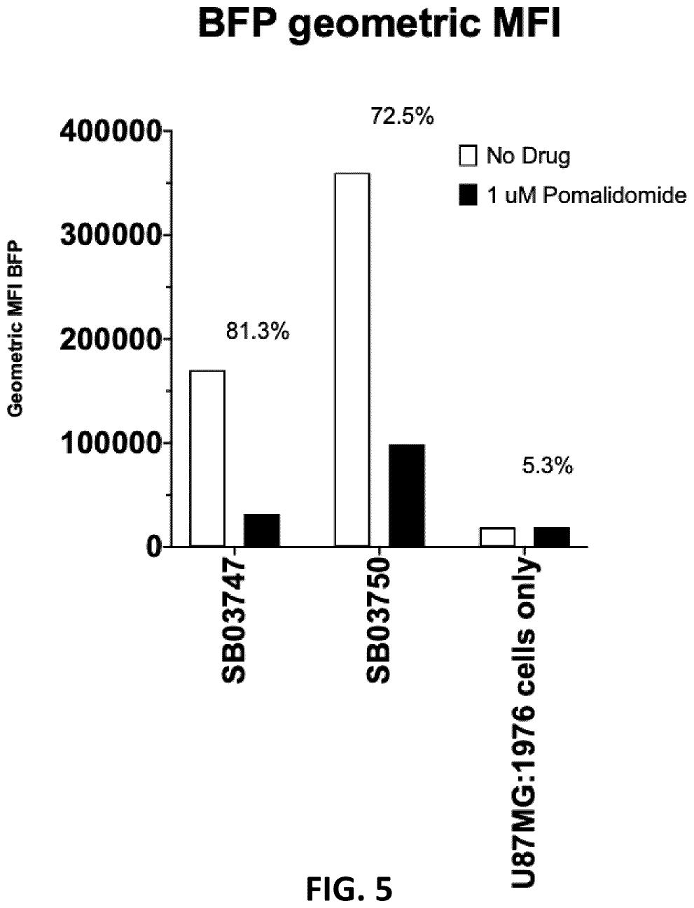

As shown in FIG. 5, constructs SB03747 and SB03750 exhibited 81.3% and 72.5% percent degradation, respectively, upon treatment with 1 μM pomalidomide. Thus, both constructs are efficiently degraded following pomalidomide treatment.

### Example 4: Degradation of Degron-Linked Repressors with Various Construct Orientations Induces Transcription

The ability of degron-linked repressors with various orientations to modulate reporter expression was assessed by transduction assay.

### Materials and Methods

A reporter cell line was produced as described in Example 2. Reporter cells were transduced (50,000 cells/transduction) with lentivirus (50,000 pg each, as titered by p24 assay) encoding constructs either having the degron N-terminal to the inducible transcription modulator (ITM) (Table 8) or having the degron C-terminal to the ITM (Table 9). Constructs SB03758 and SB03759 as described in Example 2 were also included in this experiment. Each construct format is described from 5′ to 3′ (ORF from N to C terminus).

TABLE 8
Constructs w/N-Term Degron

| Construct | |
|---|---|
| Name | Construct Description |
| SB03953 | degron-A(EAAAK)3A-SCX-ZF10 |
| SB03954 | degron-A(EAAAK)3A-ID1-ZF10 |
| SB03955 | degron-A(EAAAK)3A-HERC2-ZF10 |
| SB03956 | degron-A(EAAAK)3A-TWST1-ZF10 |
| SB03957 | degron-A(EAAAK)3A-NKX22-ZF10 |
| SB03958 | degron-A(EAAAK)3A-ID3-ZF10 |
| SB03959 | degron-A(EAAAK)3A-TWST2-ZF10 |
| SB03960 | degron-A(EAAAK)3A-KRAB_{Q354A/Q36A}-ZF10 |
| SB03961 | degron-A(EAAAK)3A-KRAB_{I62A}-ZF10 |
| SB03936 | degron-A(EAAAK)3A-HDAC4-ZF10 |

TABLE 9
Constructs w/C-Term Degron

| Construct | |
|---|---|
| Name | Construct Description |
| SB03944 | ZF10-SCX-ecpd-degron |
| SB03945 | ZF10-ID1-ecpd-degron |
| SB03946 | ZF10-HERC2-ecpd-degron |
| SB03947 | ZF10-TWST1-ecpd-degron |
| SB03948 | ZF10-NKX22-ecpd-degron |
| SB03949 | ZF10-ID3-ecpd-degron |
| SB03950 | ZF10-TWST2-ecpd-degron |
| SB03951 | ZF10-KRAB_{Q354A/Q36A}-ecpd-degron |
| SB03952 | ZF10-KRAB_{I62A}-ecpd-degron |
| SB03937 | ZF10-HDAC4-ecpd-degron |

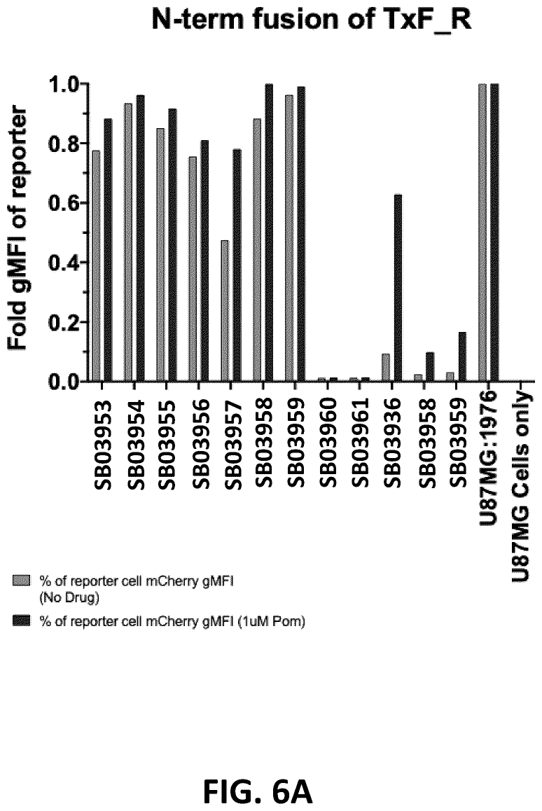

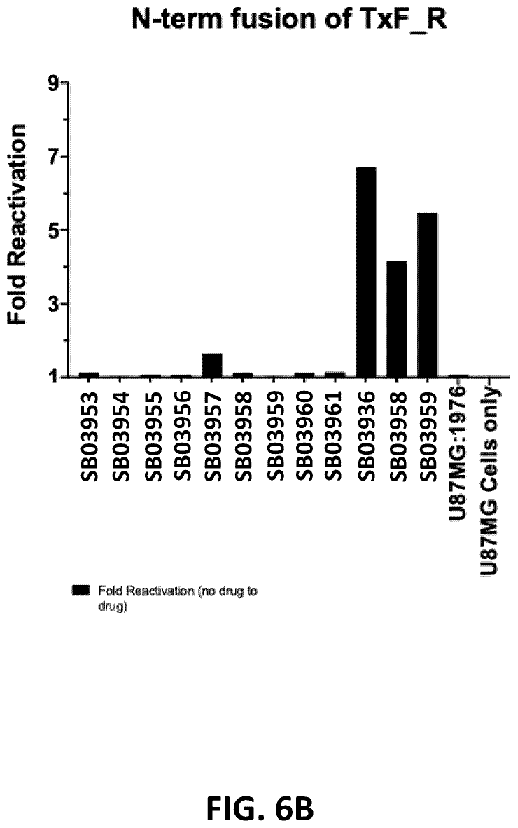

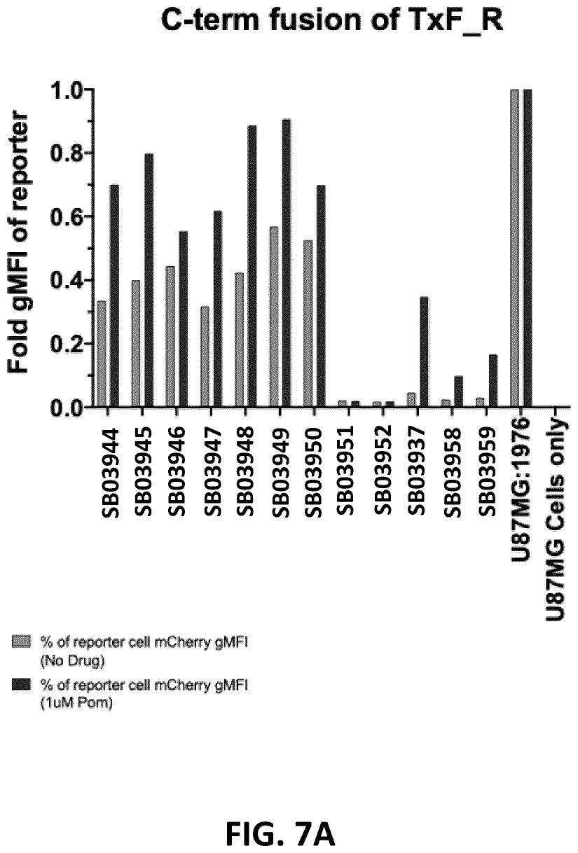

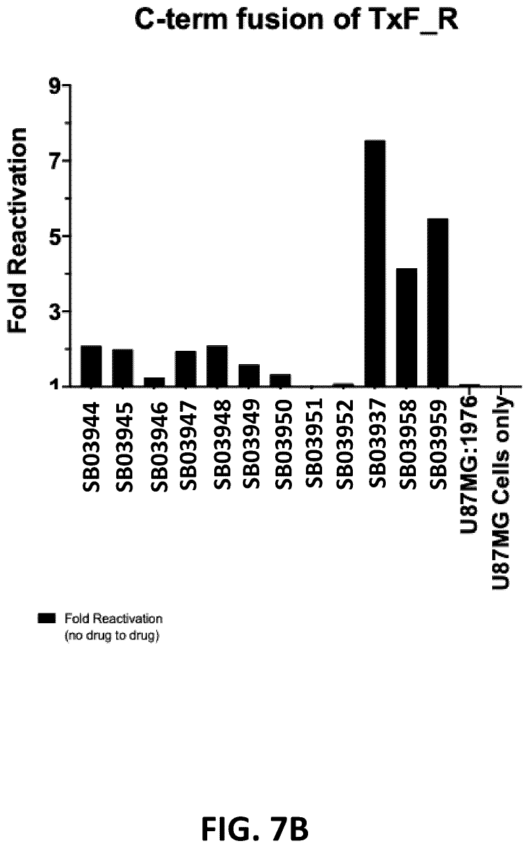

On day 2 following transduction, cells transduced with each construct were incubated either in the presence of 1 μM pomalidomide or in the absence of the drug. On day 5 of drug treatment, cells were assayed by Fluorescence-Activated Cell Sorting (FACS) for mCherry expression. The geometric mean fluorescent intensity (MFI) of mCherry for each construct having the degron at the N-terminus is shown in FIG. 6A. The fold difference between the geometric MFI of mCherry for the no drug condition versus the pomalidomide treatment condition for each construct having the degron at the N-terminus was calculated as shown in FIG. 6B. The geometric mean fluorescent intensity (MFI) of mCherry for each construct having the degron at the C-terminus is shown in FIG. 7A. The fold difference between the geometric MFI of mCherry for the no drug condition versus the pomalidomide treatment condition for each construct having the degron at the C-terminus was calculated as shown in FIG. 7B.

### Results

Degron-linked promoter regulation was assessed for constructs including a cereblon (CRBN) polypeptide substrate domain (d913) in various orientations and using various repression domains, linkers, and mRNA-destabilizing elements. As shown in FIG. 6A, the degron-linked repressor having an HDAC4 repressor domain and having the degron at the N-terminus was capable of repressing reporter expression in the absence of drug, and the repression was released as shown by the induced reporter expression in the presence of drug. Additionally, FIG. 6A confirms the reversibility of repression by constructs SB03758 and SB03759 as was demonstrated in Example 2. As shown in FIG. 6B, the degron-linked repressor having an HDAC4 repressor domain and having the degron at the N-terminus, and constructs SB03758 and SB03759 each demonstrate at least a 4-fold increase in reporter expression in the presence of drug as compared to the absence of drug. As shown in FIG. 7A, the degron-linked repressor having an HDAC4 repressor domain and having the degron at the C-terminus was capable of repressing reporter expression in the absence of drug, and the repression was reversible as shown by the induced reporter expression in the presence of drug. Additionally, FIG. 7A provides another confirmation that constructs SB03758 and SB03759 as shown in FIG. 6A and also demonstrated in Example 2. As shown in FIG. 7B, the degron-linked repressor having an HDAC4 repressor domain and having the degron at the C-terminus, and constructs SB03758 and SB03759 each demonstrate at least a 4-fold increase in reporter expression in the presence of drug as compared to the absence of drug. Accordingly, the data demonstrate constructs capable of repressing transcription in a drug-dependent, regulatable manner.

TABLE 10
Sequences

| | SEQ | |
|---|---|---|
| | ID | |
| Name | NO | Sequence |
| ZNF10 | 1 | MDAKSLTAWSRTLVTFKDVFVDFTR |
| | | EEWKLLDTAQQIVYRNVMLENYKNL |
| | | VSLGYQLTKPDVILRLEKGEEPWLV |
| | | EREIHQETHPDSETAFEIKSSVSSR |
| | | SIFKDKQSCDIKMEGMARNDLWYLS |
| | | LEEVWKCRDQLDKYQENPERHLRQV |
| | | AFTQKKVLTQERVSESGKYGGNCLL |
| | | PAQLVLREYFHKRDSHTKSLKHDLV |
| | | LNGHQDSCASNSNECGQTFCQNIHL |
| | | IQFARTHTGDKSYKCPDNDNSLTHG |
| | | SSLGISKGIHREKPYECKECGKFFS |
| | | WRSNLTRHQLIHTGEKPYECKECGK |
| | | SFSRSSHLIGHQKTHTGEEPYECKE |
| | | CGKSFSWFSHLVTHQRTHTGDKLYT |
| | | CNQCGKSFVHSSRLIRHQRTHTGEK |
| | | PYECPECGKSFRQSTHLILHQRTHV |
| | | RVRPYECNECGKSYSQRSHLVVHHR |
| | | IHTGLKPFECKDCGKCFSRSSHLYS |
| | | HQRTHTGEKPYECHDCGKSFSQSSA |
| | | LIVHQRIHTGEKPYECCQCGKAFIR |
| | | KNDLIKHQRIHVGEETYKCNQCGII |
| | | FSQNSPFIVHQIAHTGEQFLTCNQC |
| | | GTALVNTSNLIGYQTNHIRENAY |
| ZNF10 | 2 | DAKSLTAWSRTLVTFKDVFVDFTRE |
| KRAB | | EWKLLDTAQQIVYRNVMLENYKNLV |
| domain | | SLGYQLTKPDVILRLEKGEEPWLVE |
| | | REIHQE |
| minKRAB | 3 | RTLVTFKDVFVDFTREEWKLLDTAQ |
| | | QIVYRNVMLENYKNLVSLGY |
| HDAC4 | 4 | SSQSHPDGLSGRDQPVELLNPARVN |
| repressor | | HMPSTVDVATALPLQVAPSAVPMDL |
| domain | | RLDHQFSLPVAEPALREQQLQQELL |
| | | ALKQKQQIQRQILIAEFQRQHEQLS |
| | | RQHEAQLHEHIKQQQEMLAMKHQQE |
| | | LLEHQRKLERHRQEQELEKQHREQK |
| | | LQQLKNKEKGKESAVASTEVKMKLQ |
| | | EFVLNKKKALAHRNLNHCISSDPRY |
| | | WYGKTQHSSLDQSSPPQSGVSTSYN |
| | | HPVLGMYDAKDDFPLRKTASEPNLK |
| | | LRSRLKQKVAERRSSPLLRRKDGPV |
| | | VTALKKRPLDVTDSACSSAPGSGPS |
| | | SPNNSSGSVSAENGIAPAVPSIPAE |
| | | TSLAHRLVAREGSAAPLPLYTSPSL |
| | | PNITLGLPATGPSAGTAGQQDTERL |
| | | TLPALQQRLSLFPGTHLTPYLSTSP |
| | | LERDGGAAHSPLLQHMVLLEQPPAQ |
| | | APLVTGLGALPLHAQSLVGADRVSP |
| | | SIHKLRQHRPLGRTQSAPLPQNAQA |
| | | LQHLVIQQQHQQFLEKHKQQFQQQQ |
| | | LQMNKIIPKPSEPARQPESHPEETE |
| | | EELREHQALLDEPYLDRLPGQKEAH |
| | | AQAGVQVKQEPIESDEEEAEPPREV |
| | | EPGQRQPSEQELLFRQQALLLEQQR |
| | | IHQLRNYQASMEAAGIPVSFGGHRP |
| | | LSRAQSSPASATFPVSVQEPPTKPR |
| | | FTTGLVYDTLMLKHQCTCGSSSSHP |
| | | EHAGRIQSIWSRLQETGLRGKCECI |
| | | RGRKATLEELQTVHSEAHTLLYGTN |
| | | PLNRQKLDSKKLLGSLASVFVRLPC |
| | | GGVGVDSDTIWNEVHSAGAARLAVG |
| | | CVVELVFKVATGELKNGFAVVRPPG |
| | | HHAEESTPMGFCYFNSVAVAAKLLQ |
| | | QRLSVSKILIVDWDVHHGNGTQQAF |
| | | YSDPSVLYMSLHRYDDGNFFPGSGA |
| | | PDEVGTGPGVGFNVNMAFTGGLDPP |
| | | MGDAEYLAAFRTVVMPIASEFAPDV |
| | | VLVSSGFDAVEGHPTPLGGYNLSAR |
| | | CFGYLTKQLMGLAGGRIVLALEGGH |
| | | DLTAICDASEACVSALLGNELDPLP |
| | | EKVLQQRPNANAVRSMEKVMEIHSK |
| | | YWRCLQRTTSTAGRSLIEAQTCENE |
| | | EAETVTAMASLSVGVKPAEKRPDEE |
| | | PMEEEPPL |
| ZF Protein | 5 | SRPGERPFQCRICMRNFSRRHGLDR |
| Domain | | HTRTHTGEKPFQCRICMRNFSDHSS |
| (ZF10) | | LKRHLRTHTGSQKPFQCRICMRNFS |
| | | VRHNLTRHLRTHTGEKPFQCRICMR |
| | | NFSDHSNLSRHLKTHTGSQKPFQCR |
| | | ICMRNFSQRSSLVRHLRTHTGEKPF |
| | | QCRICMRNFSESGHLKRHLRTHLRG |
| | | S |
| CRBN | 6 | FNVLMVHKRSHTGERPLQCEICGFT |
| polypeptide | | CRQKGNLLRHIKLHTGEK |
| | | PFKCHLCNYACQRRDAL |
| substrate | | |
| domain | | |
| (degron) | | |
| Peptide | 7 | GSGSGSGS |
| Linker | | |
| Peptide | 8 | KEGS |
| Linker | | |
| Peptide | 9 | EAAAK |
| Linker | | |
| Peptide | 10 | AAPAKQE |
| Linker | | |
| Peptide | 11 | GSGSGSGSGGAEAAAKEAAAKEAAA |
| Linker | | KA |
| (ConMJ) | | |
| Peptide | 12 | AAPAKQEAAAPAKQEAAAPAKQEAA |
| Linker (ecpd) | | APAPAAKAEAPAAAPAAKA |
| Peptide | 13 | AEAAAKEAAAKEAAAKA |
| Linker | | |
| AU-rich motif | 14 | ATTTA |
| AU-rich | 15 | ATTTATTTATTTATTTATTTA |
| element | | |
| SLDE | 16 | CTGTTTAATATTTAAACAG |
| AuSLDE | 17 | ATTTATTTATTTATTTATTTAacat |
| | | cggttccCTGTTTAATATTTAAACA |
| | | G |
| 2X AuSLDE | 18 | ATTTATTTATTTATTTATTTAacat |
| | | cggttccCTGTTTAATATTTAAACA |
| | | GtgcggtaagcATTTATTTATTTAT |
| | | TTATTTAacatcggttccCTGTTTA |
| | | ATATTTAAACAG |
| minimal | 19 | AGAGGGTATATAATGGAAGCTCGAC |
| promoter; | | TTCCAG |
| minP | | |
| NFkB | 20 | GGGAATTTCCGGGGACTTTCCGGGA |
| response | | ATTTCCGGGGACTTTCCGGGAATTT |
| element | | CC |
| protein | | |
| promoter; 5x | | |
| NFkB-RE | | |
| CREB | 21 | CACCAGACAGTGACGTCAGCTGCCA |
| response | | GATCCCATGGCCGTCATACTGTGAC |
| element | | GTCTTTCAGACACCCCATTGACGTC |
| protein | | AATGGGAGAA |
| promoter; 4x | | |
| CRE | | |
| NFAT | 22 | GGAGGAAAAACTGTTTCATACAGAA |
| response | | GGCGTGGAGGAAAAACTGTTTCATA |
| element | | CAGAAGGCGTGGAGGAAAAACTGTT |
| protein | | TCATACAGAAGGCGT |
| promoter; 3x | | |
| NFAT | | |
| binding sites | | |
| SRF response | 23 | AGGATGTCCATATTAGGACATCTAG |
| element | | GATGTCCATATTAGGACATCTAGGA |
| | | TGTCCATATTAGGACATCTAGGATG |
| | | TCCATATT |
| protein | | AGGACATCTAGGATGTCCATATTAG |
| promoter; 5x | | GACATCT |
| SRE | | |
| SRF response | 24 | AGTATGTCCATATTAGGACATCTAC |
| element | | CATGTCCATATTAGGACATCTACTA |
| protein | | TGTCCATATTAGGACATCTTGTATG |
| promoter 2; | | TCCATATTAGGACATCTAAAATGTC |
| 5x SRF-RE | | CATATTAGGACATCT |
| API response | 25 | TGAGTCAGTGACTCAGTGAGTCAGT |
| element | | GACTCAGTGAGTCAGTGACTCAG |
| protein | | |
| promoter; 6x | | |
| AP1-RE | | |
| TCF-LEF | 26 | AGATCAAAGGGTTTAAGATCAAAGG |
| response | | GCTTAAGATCAAAGGGTATAAGATC |
| element | | AAAGGGCCTAAGATCAAAGGGACTA |
| promoter; 8x | | AGATCAAAGGGTTTAAGATCAAAGG |
| TCF-LEF-RE | | GCTTAAGATCAAAGGGCCTA |
| SBEx4 | 27 | GTCTAGACGTCTAGACGTCTAGACG |
| | | TCTAGAC |
| SMAD2/3- | 28 | CAGACACAGACACAGACACAGACA |
| CAGACA x4 | | |
| STAT3 | 29 | GGATCCGGTACTCGAGATCTGCGAT |
| binding site | | CTAAGTAAGCTTGGCATTCCGGTAC |
| | | TGTTGGTAAAGCCAC |
| CMV | 30 | GTTGACATTGATTATTGACTAGTTA |
| | | TTAATAGTAATCAATTACGGGGTCA |
| | | TTAGTTCATAGCCCATATATGGAGT |
| | | TCCGCGTTACATAACTTACGGTAAA |
| | | TGGCCCGCCTGGCTGACCGCCCAAC |
| | | GACCCCCGCCCATTGACGTCAATAA |
| | | TGACGTATGTTCCCATAGTAACGCC |
| | | AATAGGGACTTTCCATTGACGTCAA |
| | | TGGGTGGAGTATTTACGGTAAACTG |
| | | CCCACTTGGCAGTACATCAAGTGTA |
| | | TCATATGCCAAGTACGCCCCCTATT |
| | | GACGTCAATGACGGTAAATGGCCCG |
| | | CCTGGCATTATGCCCAGTACATGAC |
| | | CTTATGGGACTTTCCTACTTGGCAG |
| | | TACATCTACGTATTAGTCATCGCTA |
| | | TTACCATGGTGATGCGGTTTTGGCA |
| | | GTACATCAATGGGCGTGGATAGCGG |
| | | TTTGACTCACGGGGATTTCCAAGTC |
| | | TCCACCCCATTGACGTCAATGGGAG |
| | | TTTGTTTTGGCACCAAAATCAACGG |
| | | GACTTTCCAAAATGTCGTAACAACT |
| | | CCGCCCCATTGACGCAAATGGGCGG |
| | | TAGGCGTGTACGGTGGGAGGTCTAT |
| | | ATAAGCAGAGCTC |
| EF1a | 31 | GGCTCCGGTGCCCGTCAGTGGGCAG |
| | | AGCGCACATCGCCCACAGTCCCCGA |
| | | GAAGTTGGGGGGAGGGGTCGGCAAT |
| | | TGAACCGGTGCCTAGAGAAGGTGGC |
| | | GCGGGGTAAACTGGGAAAGTGATGC |
| | | CGTGTACTGGCTCCGCCTTTTTCCC |
| | | GAGGGTGGGGGAGAACCGTATATAA |
| | | GTGCAGTAGTCGCCGTGAACGTTCT |
| | | TTTTCGCAACGGGTTTGCCGCCAGA |
| | | ACACAGGTAAGTGCCGTGTGTGGTT |
| | | CCCGCGGGCCTGGCCTCTTTACGGG |
| | | TTATGGCCCTTGCGTGCCTTGAATT |
| | | ACTTCCACCTGGCTGCAGTACGTGA |
| | | TTCTTGATCCCGAGCTTCGGGTTGG |
| | | AAGTGGGTGGGAGAGTTCGAGGCCT |
| | | TGCGCTTAAGGAGCCCCTTCGCCTC |
| | | GTGCTTGAGTTGAGGCCTGGCCTGG |
| | | GCGCTGGGGCCGCCGCGTGCGAATC |
| | | TGGTGGCACCTTCGCGCCTGTCTCG |
| | | CTGCTTTCGATAAGTCTCTAGCCAT |
| | | TTAAAATTTTTGATGACCTGCTGCG |
| | | ACGCTTTTTTTCTGGCAAGATAGTC |
| | | TTGTAAATGCGGGCCAAGATCTGCA |
| | | CACTGG |
| | | TATTTCGGTTTTTGGGGCCGCGGGC |
| | | GGCGACGGGGCCCGTGCGTCCCAGC |
| | | GCACATGTTCGGCGAGGCGGGGCCT |
| | | GCGAGCGCGACCACCGAGAATCGGA |
| | | CGGGGGTAGTCTCAAGCTGGCCGGC |
| | | CTGCTCTGGTGCCTGTCCTCGCGCC |
| | | GCCGTGTATCGCCCCGCCCCGGGCG |
| | | GCAAGGCTGGCCCGGTCGGCACCAG |
| | | TTGCGTGAGCGGAAAGATGGCCGCT |
| | | TCCCGGTCCTGCTGCAGGGAGCTCA |
| | | AAATGGAGGACGCGGCGCTCGGGAG |
| | | AGCGGGCGGGTGAGTCACCCACACA |
| | | AAGGAAAAGGGCCTTTCCGTCCTCA |
| | | GCCGTCGCTTCATGTGACTCCACGG |
| | | AGTACCGGGCGCCGTCCAGGCACCT |
| | | CGATTAGTTCTCGAGCTTTTGGAGT |
| | | ACGTCGTCTTTAGGTTGGGGGGAGG |
| | | GGTTTTATGCGATGGAGTTTCCCCA |
| | | CACTGAGTGGGTGGAGACTGAAGTT |
| | | AGGCCAGCTTGGCACTTGATGTAAT |
| | | TCTCCTTGGAATTTGCCCTTTTTGA |
| | | GTTTGGATCTTGGTTCATTCTCAAG |
| | | CCTCAGACAGTGGTTCAAAGTTTTT |
| | | TTCTTCCATTTCAGGTGTCGTGA |
| EFS | 32 | GGATCTGCGATCGCTCCGGTGCCCG |
| | | TCAGTGGGCAGAGCGCACATCGCCC |
| | | ACAGTCCCCGAGAAGTTGGGGGGAG |
| | | GGGTCGGCAATTGAACCGGTGCCTA |
| | | GAGAAGGTGGCGCGGGGTAAACTGG |
| | | GAAAGTGATGTCGTGTACTGGCTCC |
| | | GCCTTTTTCCCGAGGGTGGGGGAGA |
| | | ACCGTATATAAGTGCAGTAGTCGCC |
| | | GTGAACGTTCTTTTTCGCAACGGGT |
| | | TTGCCGCCAGAACACAGCTGAAGCT |
| | | TCGAGGGGCTCGCATCTCTCCTTCA |
| | | CGCGCCCGCCGCCCTACCTGAGGCC |
| | | GCCATCCACGCCGGTTGAGTCGCGT |
| | | TCTGCCGCCTCCCGCCTGTGGTGCC |
| | | TCCTGAACTGCGTCCGCCGTCTAGG |
| | | TAAGTTTAAAGCTCAGGTCGAGACC |
| | | GGGCCTTTGTCCGGCGCTCCCTTGG |
| | | AGCCTACCTAGACTCAGCCGGCTCT |
| | | CCACGCTTTGCCTGACCCTGCTTGC |
| | | TCAACTCTACGTCTTTGTTTCGTTT |
| | | TCTGTTCTGCGCCGTTACAGATCCA |
| | | AGCTGTGACCGGCGCCTAC |
| MND | 33 | TTTATTTAGTCTCCAGAAAAAGGGG |
| | | GGAATGAAAGACCCCACCTGTAGGT |
| | | TTGGCAAGCTAGGATCAAGGTTAGG |
| | | AACAGAGAGACAGCAGAATATGGGC |
| | | CAAACAGGATATCTGTGGTAAGCAG |
| | | TTCCTGCCCCGGCTCAGGGCCAAGA |
| | | ACAGTTGGAACAGCAGAATATGGGC |
| | | CAAACAGGATATCTGTGGTAAGCAG |
| | | TTCCTGCCCCGGCTCAGGGCCAAGA |
| | | ACAGATGGTCCCCAGATGCGGTCCC |
| | | GCCCTCAGCAGTTTCTAGAGAACCA |
| | | TCAGATGTTTCCAGGGTGCCCCAAG |
| | | GACCTGAAATGACCCTGTGCCTTAT |
| | | TTGAACTAACCAATCAGTTCGCTTC |
| | | TCGCTTCTGTTCGCGCGCTTCTGCT |
| | | CCCCGAGCTCAATAAAAGAGCCCA |
| PGK | 34 | GGGGTTGGGGTTGCGCCTTTTCCAA |
| | | GGCAGCCCTGGGTTTGCGCAGGGAC |
| | | GCGGCTGCTCTGGGCGTGGTTCCGG |
| | | GAAACGCAGCGGCGCCGACCCTGGG |
| | | TCTCGCACATTCTTCACGTCCGTTC |
| | | GCAGCGTCACCCGGATCTTCGCCGC |
| | | TACCCTTGTGGGCCCCCCGGCGACG |
| | | CTTCCTGCTCCGCCCCTAAGTCGGG |
| | | AAGGTTCCTTGCGGTTCGCGGCGTG |
| | | CCGGACGTGACAAACGGAAGCCGCA |
| | | CGTCTCACTAGTACCCTCGCAGACG |
| | | GACAGCGCCAGGGAGCAATGGCAGC |
| | | GCGCCGACCGCGATGGGCTGTGGCC |
| | | AATAGCGGCTGCTCAGCGGGGCGCG |
| | | CCGAGAGCAGCGGCCGGGAAGGGGC |
| | | GGTGCGGGAGGCGGGGTGTGGGGCG |
| | | GTAGTGTGGGCCCTGTTCCTGCCCG |
| | | CGCGGTGTTCCGCATTCTGCAAGCC |
| | | TCCGGAGCGCACGTCGGCAGTCGGC |
| | | TCCCTCGTTGACCGAATCACCGACC |
| | | TCTCTCCCCAG |
| SFFV | 35 | GTAACGCCATTTTGCAAGGCATGGA |
| | | AAAATACCAAACCAAGAATAGAGAA |
| | | GTTCAGATCAAGGGCGGGTACATGA |
| | | AAATAGCTAACGTTGGGCCAAACAG |
| | | GATATCTGCGGTGAGCAGTTTCGGC |
| | | CCCGGCCCGGGGCCAAGAACAGATG |
| | | GTCACCGCAGTTTCGGCCCCGGCCC |
| | | GAGGCCAAGAACAGATGGTCCCCAG |
| | | ATATGGCCCAACCCTCAGCAGTTTC |
| | | TTAAGACCCATCAGATGTTTCCAGG |
| | | CTCCCCCAAGGACCTGAAATGACCC |
| | | TGCGCCTTATTTGAATTAACCAATC |
| | | AGCCTGCTTCTCGCTTCTGTTCGCG |
| | | CGCTTCTGCTTCCCGAGCTCTATAA |
| | | AAGAGCTCACAACCCCTCACTCGGC |
| | | GCGCCAGTCCTCCGACAGACTGAGT |
| | | CGCCCGGG |
| SV40 | 36 | CTGTGGAATGTGTGTCAGTTAGGGT |
| | | GTGGAAAGTCCCCAGGCTCCCCAGC |
| | | AGGCAGAAGTATGCAAAGCATGCAT |
| | | CTCAATTAGTCAGCAACCAGGTGTG |
| | | GAAAGTCCCCAGGCTCCCCAGCAGG |
| | | CAGAAGTATGCAAAGCATGCATCTC |
| | | AATTAGTCAGCAACCATAGTCCCGC |
| | | CCCTAACTCCGCCCATCCCGCCCCT |
| | | AACTCCGCCCAGTTCCGCCCATTCT |
| | | CCGCCCCATGGCTGACTAATTTTTT |
| | | TTATTTATGCAGAGGCCGAGGCCGC |
| | | CTCTGCCTCTGAGCTATTCCAGAAG |
| | | TAGTGAGGAGGCTTTTTTGGAGGCC |
| | | TAGGCTTTTGCAAAAAGCT |
| UbC | 37 | GCGCCGGGTTTTGGCGCCTCCCGCG |
| | | GGCGCCCCCCTCCTCACGGCGAGCG |
| | | CTGCCACGTCAGACGAAGGGCGCAG |
| | | GAGCGTTCCTGATCCTTCCGCCCGG |
| | | ACGCTCAGGACAGCGGCCCGCTGCT |
| | | CATAAGACTCGGCCTTAGAACCCCA |
| | | GTATCAGCAGAAGGACATTTTAGGA |
| | | CGGGACTTGGGTGACTCTAGGGCAC |
| | | TGGTTTTCTTTCCAGAGAGCGGAAC |
| | | AGGCGAGGAAAAGTAGTCCCTTCTC |
| | | GGCGATTCTGCGGAGGGATCTCCGT |
| | | GGGGCGGTGAACGCCGATGATTATA |
| | | TAAGGACGCGCCGGGTGTGGCACAG |
| | | CTAGTTCCGTCGCAGCCGGGATTTG |
| | | GGTCGCGGTTCTTGTTTGTGGATCG |
| | | CTGTGATCGTCACTTGGTGAGTTGC |
| | | GGGCTGCTGGGCTGGCCGGGGCTTT |
| | | CGTGGCCGCCGGGCCGCTCGGTGGG |
| | | ACGGAAGCGTGTGGAGAGACCGCCA |
| | | AGGGCTGTAGTCTGGGTCCGCGAGC |
| | | AAGGTTGCCCTGAACTGGGGGTTGG |
| | | GGGGAGCGCACAAAATGGCGGCTGT |
| | | TCCCGAGTCTTGAATGGAAGACGCT |
| | | TGTAAGGCGGGCTGTGAGGTCGTTG |
| | | AAACAAGGTGGGGGGCATGGTGGGC |
| | | GGCAAGAACCCAAGGTCTTGAGGCC |
| | | TTCGCTAATGCGGGAAAGCTCTTAT |
| | | TCGGGTGAGATGGGCTGGGGCACCA |
| | | TCTGGGGACCCTGACGTGAAGTTTG |
| | | TCACTGACTGGAGAACTCGGGTTTG |
| | | TCGTCTGGTTGCGGGGGCGGCAGTT |
| | | ATGCGGTGCCGTTGGGCAGTGCACC |
| | | CGTACCTTTGGGAGCGCGCGCCTCG |
| | | TCGTGTCGTGACGTCACCCGTTCTG |
| | | TTGGCTTATAATGCAGGGTGGGGCC |
| | | ACCTGCCGGTAGGTGTGCGGTAGGC |
| | | TTTTCTCCGTCGCAGGACGCAGGGT |
| | | TCGGGCCTAGGGTAGGCTCTCCTGA |
| | | ATCGACAGGCGCCGGACCTCTGGTG |
| | | AGGGGAGGGATAAGTGAGGCGTCAG |
| | | TTTCTTTGGTCGGTTTTATGTACCT |
| | | ATCTTCTTAAGTAGCTGAAGCTCCG |
| | | GTTTTGAACTATGCGCTCGGGGTTG |
| | | GCGAGTGTGTTTTGTGAAGTTTTTT |
| | | AGGCACCTTTTGAAATGTAATCATT |
| | | TGGGTCAATATGTAATTTTCAGTGT |
| | | TAGACTAGTAAAGCTTCTGCAGGTC |
| | | GACTCTAGAAAATTGTCCGCTAAAT |
| | | TCTGGCCGTTTTTGGCTTTTTTGTT |
| | | AGAC |
| hEF1aV1 | 38 | GGCTCCGGTGCCCGTCAGTGGGCAG |
| | | AGCGCACATCGCCCACAGTCCCCGA |
| | | GAAGTTGGGGGGAGGGGTCGGCAAT |
| | | TGAACCGGTGCCTAGAGAAGGTGGC |
| | | GCGGGGTAAACTGGGAAAGTGATGT |
| | | CGTGTACTGGCTCCGCCTTTTTCCC |
| | | GAGGGTGGGGGAGAACCGTATATAA |
| | | GTGCAGTAGTCGCCGTGAACGTTCT |
| | | TTTTCGCAACGGGTTTGCCGCCAGA |
| | | ACACAGGTAAGTGCCGTGTGTGGTT |
| | | CCCGCGGGCCTGGCCTCTTTACGGG |
| | | TTATGGCCCTTGCGTGCCTTGAATT |
| | | ACTTCCACCTGGCTGCAGTACGTGA |
| | | TTCTTGATCCCGAGCTTCGGGTTGG |
| | | AAGTGGGTGGGAGAGTTCGAGGCCT |
| | | TGCGCTTAAGGAGCCCCTTCGCCTC |
| | | GTGCTTGAGTTGAGGCCTGGCCTGG |
| | | GCGCTGGGGCCGCCGCGTGCGAATC |
| | | TGGTGGCACCTTCGCGCCTGTCTCG |
| | | CTGCTTTCGATAAGTCTCTAGCCAT |
| | | TTAAAATTTTTGATGACCTGCTGCG |
| | | ACGCTTTTTTTCTGGCAAGATAGTC |
| | | TTGTAAATGCGGGCCAAGATCTGCA |
| | | CACTGGTATTTCGGTTTTTGGGGCC |
| | | GCGGGCGGCGACGGGGCCCGTGCGT |
| | | CCCAGCGCACATGTTCGGCGAGGCG |
| | | GGGCCTGCGAGCGCGGCCACCGAGA |
| | | ATCGGACGGGGGTAGTCTCAAGCTG |
| | | GCCGGCCTGCTCTGGTGCCTGGTCT |
| | | CGCGCCGCCGTGTATCGCCCCGCCC |
| | | TGGGCGGCAAGGCTGGCCCGGTCGG |
| | | CACCAGTTGCGTGAGCGGAAAGATG |
| | | GCCGCTTCCCGGCCCTGCTGCAGGG |
| | | AGCTCAAAATGGAGGACGCGGCGCT |
| | | CGGGAGAGCGGGCGGGTGAGTCACC |
| | | CACACAAAGGAAAAGGGCCTTTCCG |
| | | TCCTCAGCCGTCGCTTCATGTGACT |
| | | CCACGGAGTACCGGGCGCCGTCCAG |
| | | GCACCTCGATTAGTTCTCGAGCTTT |
| | | TGGAGTACGTCGTCTTTAGGTTGGG |
| | | GGGAGGGGTTTTATGCGATGGAGTT |
| | | TCCCCACACTGAGTGGGTGGAGACT |
| | | GAAGTTAGGCCAGCTTGGCACTTGA |
| | | TGTAATTCTCCTTGGAATTTGCCCT |
| | | TTTTGAGTTTGGATCTTGGTTCATT |
| | | CTCAAGCCTCAGACAGTGGTTCAAA |
| | | GTTTTTTTCTTCCATTTCAGGTGTC |
| | | GTGA |
| hCAGG | 39 | ACTAGTTATTAATAGTAATCAATTA |
| | | CGGGGTCATTAGTTCATAGCCCATA |
| | | TATGGAGTTCCGCGTTACATAACTT |
| | | ACGGTAAATGGCCCGCCTGGCTGAC |
| | | CGCCCAACGACCCCCGCCCATTGAC |
| | | GTCAATAATGACGTATGTTCCCATA |
| | | GTAACGCCAATAGGGACTTTCCATT |
| | | GACGTCAATGGGTGGAGTATTTACG |
| | | GTAAACTGCCCACTTGGCAGTACAT |
| | | CAAGTGTATCATATGCCAAGTACGC |
| | | CCCCTATTGACGTCAATGACGGTAA |
| | | ATGGCCCGCCTGGCATTATGCCCAG |
| | | TACATGACCTTATGGGACTTTCCTA |
| | | CTTGGCAGTACATCTACGTATTAGT |
| | | CATCGCTATTACCATGGTCGAGGTG |
| | | AGCCCCACGTTCTGCTTCACTCTCC |
| | | CCATCTCCCCCCCCTCCCCACCCCC |
| | | AATTTTGTATTTATTTATTTTTTAA |
| | | TTATTTTGTGCAGCGATGGGGGCGG |
| | | GGGGGGGGGGGGGGCGCGCGCCAGG |
| | | CGGGGCGGGGGGGGCGAGGGGCGGG |
| | | GCGGGGCGAGGCGGAGAGGTGCGGC |
| | | GGCAGCCAATCAGAGCGGCGCGCTC |
| | | CGAAAGTTTCCTTTTATGGCGAGGC |
| | | GGCGGCGGCGGCGGCCCTATAAAAA |
| | | GCGAAGCGCGCGGCGGGGGGGAGTC |
| | | GCTGCGACGCTGCCTTCGCCCCGTG |
| | | CCCCGCTCCGCCGCCGCCTCGCGCC |
| | | GCCCGCCCCGGCTCTGACTGACCGC |
| | | GTTACTCCCACAGGTGAGCGGGCGG |
| | | GACGGCCCTTCTCCTCCGGGCTGTA |
| | | ATTAGCGCTTGGTTTAATGACGGCT |
| | | TGTTTCTTTTCTGTGGCTGCGTGAA |
| | | AGCCTTGAGGGGCTCCGGGAGGGCC |
| | | CTTTGTGCGGGGGGAGCGGCTCGGG |
| | | GGGTGCGTGCGTGTGTGTGTGCGTG |
| | | GGGAGCGCCGCGTGCGGCTCCGCGC |
| | | TGCCCGGCGGCTGTGAGCGCTGCGG |
| | | GCGCGGCGCGGGGCTTTGTGCGCTC |
| | | CGCAGTGTGCGCGAGGGGAGCGCGG |
| | | CCGGGGGCGGTGCCCCGCGGTGCGG |
| | | GGGGGGCTGCGAGGGGAACAAAGGC |
| | | TGCGTGCGGGGTGTGTGC |
| | | GTGGGGGGGTGAGCAGGGGGTGTGG |
| | | GCGCGTCGGTCGGGCTGCAACCCCC |
| | | CCTGCACCCCCCTCCCCGAGTTGCT |
| | | GAGCACGGCCCGGCTTCGGGTGCGG |
| | | GGCTCCGTACGGGGCGTGGCGCGGG |
| | | GCTCGCCGTGCCGGGCGGGGGGTGG |
| | | CGGCAGGTGGGGGTGCCGGGCGGGG |
| | | CGGGGCCGCCTCGGGCCGGGGAGGG |
| | | CTCGGGGGAGGGGCGCGGCGGCCCC |
| | | CGGAGCGCCGGCGGCTGTCGAGGCG |
| | | CGGCGAGCCGCAGCCATTGCCTTTT |
| | | ATGGTAATCGTGCGAGAGGGCGCAG |
| | | GGACTTCCTTTGTCCCAAATCTGTG |
| | | CGGAGCCGAAATCTGGGAGGCGCCG |
| | | CCGCACCCCCTCTAGCGGGCGCGGG |
| | | GCGAAGCGGTGCGGCGCCGGCAGGA |
| | | AGGAAATGGGCGGGGAGGGCCTTCG |
| | | TGCGTCGCCGCGCCGCCGTCCCCTT |
| | | CTCCCTCTCCAGCCTCGGGGCTGTC |
| | | CGCGGGGGGACGGCTGCCTTCGGGG |
| | | GGGACGGGGCAGGGCGGGGTTCGGC |
| | | TTCTGGCGTGTGACCGGCGGCTCTA |
| | | GAGCCTCTGCTAACCATGTTCATGC |
| | | CTTCTTCTTTTTCCTACAGCTCCTG |
| | | GGCAACGTGCTGGTTATTGTGCTGT |
| | | CTCATCATTTTGGCAAAGAATTC |
| hEFlaV2 | 40 | GGGCAGAGCGCACATCGCCCACAGT |
| | | CCCCGAGAAGTTGGGGGGAGGGGTC |
| | | GGCAATTGAACCGGTGCCTAGAGAA |
| | | GGTGGCGCGGGGTAAACTGGGAAAG |
| | | TGATGTCGTGTACTGGCTCCGCCTT |
| | | TTTCCCGAGGGTGGGGGAGAACCGT |
| | | ATATAAGTGCAGTAGTCGCCGTGAA |
| | | CGTTCTTTTTCGCAACGGGTTTGCC |
| | | GCCAGAACACAG |
| hACTb | 41 | CCACTAGTTCCATGTCCTTATATGG |
| | | ACTCATCTTTGCCTATTGCGACACA |
| | | CACTCAATGAACACCTACTACGCGC |
| | | TGCAAAGAGCCCCGCAGGCCTGAGG |
| | | TGCCCCCACCTCACCACTCTTCCTA |
| | | TTTTTGTGTAAAAATCCAGCTTCTT |
| | | GTCACCACCTCCAAGGAGGGGGAGG |
| | | AGGAGGAAGGCAGGTTCCTCTAGGC |
| | | TGAGCCGAATGCCCCTCTGTGGTCC |
| | | CACGCCACTGATCGCTGCATGCCCA |
| | | CCACCTGGGTACACACAGTCTGTGA |
| | | TTCCCGGAGCAGAACGGACCCTGCC |
| | | CACCCGGTCTTGTGTGCTACTCAGT |
| | | GGACAGACCCAAGGCAAGAAAGGGT |
| | | GACAAGGACAGGGTCTTCCCAGGCT |
| | | GGCTTTGAGTTCCTAGCACCGCCCC |
| | | GCCCCCAATCCTCTGTGGCACATGG |
| | | AGTCTTGGTCCCCAGAGTCCCCCAG |
| | | CGGCCTCCAGATGGTCTGGGAGGGC |
| | | AGTTCAGCTGTGGCTGCGCATAGCA |
| | | GACATACAACGGACGGTGGGCCCAG |
| | | ACCCAGGCTGTGTAGACCCAGCCCC |
| | | CCCGCCCCGCAGTGCCTAGGTCACC |
| | | CACTAACGCCCCAGGCCTGGTCTTG |
| | | GCTGGGCGTGACTGTTACCCTCAAA |
| | | AGCAGGCAGCTCCAGGGTAAAAGGT |
| | | GCCCTGCCCTGTAGAGCCCACCTTC |
| | | CTTCCCAGGGCTGCGGCTGGGTAGG |
| | | TTTGTAGCCTTCATCACGGGCCACC |
| | | TCCAGCCACTGGACCGCTGGCCCCT |
| | | GCCCTGTCCTGGGGAGTGTGGTCCT |
| | | GCGACTTCTAAGTGGCCGCAAGCCA |
| | | CCTGACTCCCCCAACACCACACTCT |
| | | ACCTCTCAAGCCCAGGTCTCTCCCT |
| | | AGTGACCCACCCAGCACATTTAGCT |
| | | AGCTGAGCCCCACAGCCAGAGGTCC |
| | | TCAGGCCCTGCTTTCAGGGCAGTTG |
| | | CTCTGAAGTCGGCAAGGGGGAGTGA |
| | | CTGCCTGGCCACTCCATGCCCTCCA |
| | | AGAGCTCCTTCTGCAGGAGCGTACA |
| | | GAACCCAGGGCCCTGGCACCCGTGC |
| | | AGACCCTGGCCCACCCCACCTGGGC |
| | | GCTCAGTGCCCAAGAGATGTCCACA |
| | | CCTAGGATGTCCCGCGGTGGGTGGG |
| | | GGGCCCGAGAGACGGGCAGGCCGGG |
| | | GGCAGGCCTGGCCATGCGGGGCCGA |
| | | ACCGGGCACTGCCCAGCGTGGGGCG |
| | | CGGGGGCCACGGCGCGCGCCCCCAG |
| | | CCCCCGGGCCCAGCACCCCAAGGCG |
| | | GCCAACGCCAAAACTCTCCCTCCTC |
| | | CTCTTCCTCAATCTCGCTCTCGCTC |
| | | TTTTTTTTTTTCGCAAAAGGAGGGG |
| | | AGAGGGGGTAAAAAAATGCTGCACT |
| | | GTGCGGCGAAGCCGGTGAGTGAGCG |
| | | GCGCGGGGCCAATCAGCGTGCGCCG |
| | | TTCCGAAAGTTGCCTTTTATGGCTC |
| | | GAGCGGCCGCGGCGGCGCCCTATAA |
| | | AACCCAGCGGCGCGACGCGCCACCA |
| | | CCGCCGAGACCGCGTCCGCCCCGCG |
| | | AGCACAGAGCCTCGCCTTTGCCGAT |
| | | CCGCCGCCCGTCCACACCCGCCGCC |
| | | AGGTAAGCCCGGCCAGCCGACCGGG |
| | | GCAGGCGGCTCACGGCCCGGCCGCA |
| | | GGCGGCCGCGGCCCCTTCGCCCGTG |
| | | CAGAGCCGCCGTCTGGGCCGCAGCG |
| | | GGGGGCGCATGGGGGGGGAACCGGA |
| | | CCGCCGTGGGGGGCGCGGGAGAAGC |
| | | CCCTGGGCCTCCGGAGATGGGGGAC |
| | | ACCCCACGCCAGTTCGGAGGCGCGA |
| | | GGCCGCGCTCGGGAGGCGCGCTCCG |
| | | GGGGTGCCGCTCTCGGGGCGGGGGC |
| | | AACCGGCGGGGTCTTTGTCTGAGCC |
| | | GGGCTCTTGCCAATGGGGATCGCAG |
| | | GGTGGGCGCGGCGGAGCCCCCGCCA |
| | | GGCCCGGTGGGGGCTGGGGCGCCAT |
| | | TGCGCGTGCGCGCTGGTCCTTTGGG |
| | | CGCTAACTGCGTGCGCGCTGGGAAT |
| | | TGGCGCTAATTGCGCGTGCGCGCTG |
| | | GGACTCAAGGCGCTAACTGCGCGTG |
| | | CGTTCTGGGGCCCGGGGTGCCGCGG |
| | | CCTGGGCTGGGGCGAAGGCGGGCTC |
| | | GGCCGGAAGGGGTGGGGTCGCCGCG |
| | | GCTCCCGGGCGCTTGCGCGCACTTC |
| | | CTGCCCGAGCCGCTGGCCGCCCGAG |
| | | GGTGTGGCCGCTGCGTGCGCGCGCG |
| | | CCGACCCGGCGCTGTTTGAACCGGG |
| | | CGGAGGCGGGGCTGGCGCCCGGTTG |
| | | GGAGGGGGTTGGGGCCTGGCTTCCT |
| | | GCCGCGCGCCGCGGGGACGCCTCCG |
| | | ACCAGTGTTTGCCTTTTATGGTAAT |
| | | AACGCGGCCGGCCCGGCTTCCTTTG |
| | | TCCCCAATCTGGGCGCGCGCCGGCG |
| | | CCCCCTGGCGGCCTAAGGACTCGGC |
| | | GCGCCGGAAGTGGCCAGGGCGGGGG |
| | | CGACCTCGGCTCACAGCGCGCCCGG |
| | | CTAT |
| heIF4A1 | 42 | GTTGATTTCCTTCATCCCTGGCACA |
| | | CGTCCAGGCAGTGTCGAATCCATCT |
| | | CTGCTACAGGGGAAAACAAATAACA |
| | | TTTGAGTCCAGTGGAGACCGGGAGC |
| | | AGAAGTAAAGGGAAGTGATAACCCC |
| | | CAGAGCCCGGAAGCCTCTGGAGGCT |
| | | GAGACCTCGCCCCCCTTGCGTGATA |
| | | GGGCCTACGGAGCCACATGACCAAG |
| | | GCACTGTCGCCTCCGCACGTGTGAG |
| | | AGTGCAGGGCCCCAAGATGGCTGCC |
| | | AGGCCTCGAGGCCTGACTCTTCTAT |
| | | GTCACTTCCGTACCGGCGAGAAAGG |
| | | CGGGCCCTCCAGCCAATGAGGCTGC |
| | | GGGGCGGGCCTTCACCTTGATAGGC |
| | | ACTCGAGTTATCCAATGGTGCCTGC |
| | | GGGCCGGAGCGACTAGGAACTAACG |
| | | TCATGCCGAGTTGCTGAGCGCCGGC |
| | | AGGCGGGGCCGGGGCGGCCAAACCA |
| | | ATGCGATGGCCGGGGCGGAGTCGGG |
| | | CGCTCTATAAGTTGTCGATAGGCGG |
| | | GCACTCCGCCCTAGTTTCTAAGGAC |
| | | CATG |
| hGAPDH | 43 | AGTTCCCCAACTTTCCCGCCTCTCA |
| | | GCCTTTGAAAGAAAGAAAGGGGAGG |
| | | GGGCAGGCCGCGTGCAGTCGCGAGC |
| | | GGTGCTGGGCTCCGGCTCCAATTCC |
| | | CCATCTCAGTCGCTCCCAAAGTCCT |
| | | TCTGTTTCATCCAAGCGTGTAAGGG |
| | | TCCCCGTCCTTGACTCCCTAGTGTC |
| | | CTGCTGCCCACAGTCCAGTCCTGGG |
| | | AACCAGCACCGATCACCTCCCATCG |
| | | GGCCAATCTCAGTCCCTTCCCCCCT |
| | | ACGTCGGGGCCCACACGCTCGGTGC |
| | | GTGCCCAGTTGAACCAGGCGGCTGC |
| | | GGAAAAAAAAAAGCGGGGAGAAAGT |
| | | AGGGCCCGGCTACTAGCGGTTTTAC |
| | | GGGCGCACGTAGCTCAGGCCTCAAG |
| | | ACCTTGGGCTGGGACTGGCTGAGCC |
| | | TGGCGGGAGGCGGGGTCCGAGTCAC |
| | | CGCCTGCCGCCGCGCCCCCGGTTTC |
| | | TATAAATTGAGCCCGCAGCCTCCCG |
| | | CTTCGCTCTCTGCTCCTCCTGTTCG |
| | | ACAGTCAGCCGCATCTTCTTTTGCG |
| | | TCGCCAGGTGAAGACGGGCGGAGAG |
| | | AAACCCGGGAGGCTAGGGACGGCCT |
| | | GAAGGCGGCAGGGGCGGGCGCAGGC |
| | | CGGATGTGTTCGCGCCGCTGCGGGG |
| | | TGGGCCCGGGCGGCCTCCGCATTGC |
| | | AGGGGGGGGCGGAGGACGTGATGCG |
| | | GCGCGGGCTGGGCATGGAGGCCTGG |
| | | TGGGGGAGGGGAGGGGAGGCGTGGG |
| | | TGTCGGCCGGGGCCACTAGGCGCTC |
| | | ACTGTTCTCTCCCTCCGCGCAGCCG |
| | | AGCCACATCGCTGAGACAC |
| hGRP78 | 44 | AGTGCGGTTACCAGCGGAAATGCCT |
| | | CGGGGTCAGAAGTCGCAGGAGAGAT |
| | | AGACAGCTGCTGAACCAATGGGACC |
| | | AGCGGATGGGGCGGATGTTATCTAC |
| | | CATTGGTGAACGTTAGAAACGAATA |
| | | GCAGCCAATGAATCAGCTGGGGGGG |
| | | CGGAGCAGTGACGTTTATTGCGGAG |
| | | GGGGCCGCTTCGAATCGGCGGCGGC |
| | | CAGCTTGGTGGCCTGGGCCAATGAA |
| | | CGGCCTCCAACGAGCAGGGCCTTCA |
| | | CCAATCGGCGGCCTCCACGACGGGG |
| | | CTGGGGGAGGGTATATAAGCCGAGT |
| | | AGGCGACGGTGAGGTCGACGCCGGC |
| | | CAAGACAGCACAGACAGATTGACCT |
| | | ATTGGGGTGTTTCGCGAGTGTGAGA |
| | | GGGAAGCGCCGCGGCCTGTATTTCT |
| | | AGACCTGCCCTTCGCCTGGTTCGTG |
| | | GCGCCTTGTGACCCCGGGCCCCTGC |
| | | CGCCTGCAAGTCGGAAATTGCGCTG |
| | | TGCTCCTGTGCTACGGCCTGTGGCT |
| | | GGACTGCCTGCTGCTGCCCAACTGG |
| | | CTGGCAC |
| hGRP94 | 45 | TAGTTTCATCACCACCGCCACCCCC |
| | | CCGCCCCCCCGCCATCTGAAAGGGT |
| | | TCTAGGGGATTTGCAACCTCTCTCG |
| | | TGTGTTTCTTCTTTCCGAGAAGCGC |
| | | CGCCACACGAGAAAGCTGGCCGCGA |
| | | AAGTCGTGCTGGAATCACTTCCAAC |
| | | GAAACCCCAGGCATAGATGGGAAAG |
| | | GGTGAAGAACACGTTGCCATGGCTA |
| | | CCGTTTCCCCGGTCACGGAATAAAC |
| | | GCTCTCTAGGATCCGGAAGTAGTTC |
| | | CGCCGCGACCTCTCTAAAAGGATGG |
| | | ATGTGTTCTCTGCTTACATTCATTG |
| | | GACGTTTTCCCTTAGAGGCCAAGGC |
| | | CGCCCAGGCAAAGGGGCGGTCCCAC |
| | | GCGTGAGGGGCCCGCGGAGCCATTT |
| | | GATTGGAGAAAAGCTGCAAACCCTG |
| | | ACCAATCGGAAGGAGCCACGCTTCG |
| | | GGCATCGGTCACCGCACCTGGACAG |
| | | CTCCGATTGGTGGACTTCCGCCCCC |
| | | CCTCACGAATCCTCATTGGGTGCCG |
| | | TGGGTGCGTGGTGCGGCGCGATTGG |
| | | TGGGTTCATGTTTCCCGTCCCCCGC |
| | | CCGCGAGAAGTGGGGGTGAAAAGCG |
| | | GCCCGACCTGCTTGGGGTGTAGTGG |
| | | GCGGACCGCGCGGCTGGAGGTGTGA |
| | | GGATCCGAACCCAGGGGTGGGGGGT |
| | | GGAGGCGGCTCCTGCGATCGAAGGG |
| | | GACTTGAGACTCACCGGCCGCACGT |
| | | C |
| hHSP70 | 46 | GGGCCGCCCACTCCCCCTTCCTCTC |
| | | AGGGTCCCTGTCCCCTCCAGTGAAT |
| | | CCCAGAAGACTCTGGAGAGTTCTGA |
| | | GCAGGGGGCGGCACTCTGGCCTCTG |
| | | ATTGGTCCAAGGAAGGCTGGGGGGC |
| | | AGGACGGGAGGCGAAAACCCTGGAA |
| | | TATTCCCGACCTGGCAGCCTCATCG |
| | | AGCTCGGTGATTGGCTCAGAAGGGA |
| | | AAAGGCGGGTCTCCGTGACGACTTA |
| | | TAAAAGCCCAGGGGCAAGCGGTCCG |
| | | GATAACGGCTAGCCTGAGGAGCTGC |
| | | TGCGACAGTCCACTACCTTTTTCGA |
| | | GAGTGACTCCCGTTGTCCCAAGGCT |
| | | TCCCAGAGCGAACCTGTGCGGCTGC |
| | | AGGCACCGGCGCGTCGAGTTTCCGG |
| | | CGTCCGGAAGGACCGAGCTCTTCTC |
| | | GCGGATCCAGTGTTCCGTTTCCAGC |
| | | CCCCAATCTCAGAGCGGAGCCGACA |
| | | GAGAGCAGGGAACCC |
| hKINb | 47 | GCCCCACCCCCGTCCGCGTTACAAC |
| | | CGGGAGGCCCGCTGGGTCCTGCACC |
| | | GTCACCCTCCTCCCTGTGACCGCCC |
| | | ACCTGATACCCAAACAACTTTCTCG |
| | | CCCCTCCAGTCCCCAGCTCGCCGAG |
| | | CGCTTGCGGGGAGCCACCCAGCCTC |
| | | AGTTTCCCCAGCCCCGGGCGGGGCG |
| | | AGGGGCGATGACGTCATGCCGGCGC |
| | | GCGGCATTGTGGGGCGGGGCGAGGC |
| | | GGGGCGCCGGGGGGAGCAACACTGA |
| | | GACGCCATTTTCGGCGGCGGGAGCG |
| | | GCGCAGGCGGCCGAGCGGGACTGGC |
| | | TGGGTCGGCTGGGCTGCTGGTGCGA |
| | | GGAGCCGCGGGGCTGTGCTCGGCGG |
| | | CCAAGGGGACAGCGCGTGGGTGGCC |
| | | GAGGATGCTGCGGGGCGGTAGCTCC |
| | | GGCGCCCCTCGCTGGTGACTGCTGC |
| | | GCCGTGCCTCACACAGCCGAGGCGG |
| | | GCTCGGCGCACAGTCGCTGCTCCGC |
| | | GCTCGCGCCCGGCGGCGCTCCAGGT |
| | | GCTGACAGCGCGAGAGAGCGCGGCC |
| | | TCAGGAGCAACAC |
| hUBIb | 48 | TTCCAGAGCTTTCGAGGAAGGTTTC |
| | | TTCAACTCAAATTCATCCGCCTGAT |
| | | AATTTTCTTATATTTTCCTAAAGAA |
| | | GGAAGAGAAGCGCATAGAGGAGAAG |
| | | GGAAATAATTTTTTAGGAGCCTTTC |
| | | TTACGGCTATGAGGAATTTGGGGCT |
| | | CAGTTGAAAAGCCTAAACTGCCTCT |
| | | CGGGAGGTTGGGCGCGGCGAACTAC |
| | | TTTCAGCGGCGCACGGAGACGGCGT |
| | | CTACGTGAGGGGTGATAAGTGACGC |
| | | AACACTCGTTGCATAAATTTGCGCT |
| | | CCGCCAGCCCGGAGCATTTAGGGGC |
| | | GGTTGGCTTTGTTGGGTGAGCTTGT |
| | | TTGTGTCCCTGTGGGTGGACGTGGT |
| | | TGGTGATTGGCAGGATCCTGGTATC |
| | | CGCTAACAGGTACTGGCCCACAGCC |
| | | GTAAAGACCTGCGGGGGCGTGAGAG |
| | | GGGGGAATGGGTGAGGTCAAGCTGG |
| | | AGGCTTCTTGGGGTTGGGTGGGCCG |
| | | CTGAGGGGAGGGGAGGGCGAGGTGA |
| | | CGCGACACCCGGCCTTTCTGGGAGA |
| | | GTGGGCCTTGTTGACCTAAGGGGGG |
| | | CGAGGGCAGTTGGCACGCGCACGCG |
| | | CCGACAGAAACTAACAGACATTAAC |
| | | CAACAGCGATTCCGTCGCGTTTACT |
| | | TGGGAGGAAGGCGGAAAAGAGGTAG |
| | | TTTGTGTGGCTTCTGGAAACCCTAA |
| | | ATTTGGAATCCCAGTATGAGAATGG |
| | | TGTCCCTTCTTGTGTTTCAATGGGA |
| | | TTTTTACTTCGCGAGTCTTGTGGGT |
| | | TTGGTTTTGTTTTCAGTTTGCCTAA |
| | | CACCGTGCTTAGGTTTGAGGCAGAT |
| | | TGGAGTTCGGTCGGGGGAGTTTGAA |
| | | TATCCGGAACAGTTAGTGGGGAAAG |
| | | CTGTGGACGCTTGGTAAGAGAGCGC |
| | | TCTGGATTTTCCGCTGTTGACGTTG |
| | | AAACCTTGAATGACGAATTTCGTAT |
| | | TAAGTGACTTAGCCTTGTAAAATTG |
| | | AGGGGAGGCTTGCGGAATATTAACG |
| | | TATTTAAGGCATTTTGAAGGAATAG |
| | | TTGCTAATTTTGAAGAATATTAGGT |
| | | GTAAAAGCAAGAAATACAATGATCC |
| | | TGAGGTGACACGCTTATGTTTTACT |
| | | TTTAAACTAGGTCACC |
| Caspase 9 | 49 | DEADRRLLRRCRLRLVEELQVDQLW |
| | | DVLLSRELFRPHMIEDIQRAGSGSR |
| | | RDQARQLIIDLETRGSQALPLFISC |
| | | LEDTGQDMLASFLRTNRQAAKLSKP |
| | | TLENLTPVVLRPEIRKPEVLRPETP |
| | | RPVDIGSGGFGDVGALESLRGNADL |
| | | AYILSMEPCGHCLIINNVNFCRESG |
| | | LRTRTGSNIDCEKLRRRFSSLHFMV |
| | | EVKGDLTAKKMVLALLELARQDHGA |
| | | LDCCVVVILSHGCQASHLQFPGAVY |
| | | GTDGCPVSVEKIVNIFNGTSCPSLG |
| | | GKPKLFFIQACGGEQKDHGFEVAST |
| | | SPEDESPGSNPEPDATPFQEGLRTF |
| | | DQLDAISSLPTPSDIFVSYSTFPGF |
| | | VSWRDPKSGSWYVETLDDIFEQWAH |
| | | SEDLQSLLLRVANAVSVKGIYKQMP |
| | | GCFNFLRKKLFFKTS |
| Diptheria | 50 | DPDDVVDSSKSFVMENFSSYHGTKP |
| Toxin A | | GYVDSIQKGIQKPKSGTQGNYDDDW |
| (DTA) | | KGFYSTDNKYDAAGYSVDNENPLSG |
| | | KAGGVVKVTYPGLTKVLALKVDNAE |
| | | TIKKELGLSLTEPLMEQVGTEEFIK |
| | | RFGDGASRVVLSLPFAEGSSSVEYI |
| | | NNWEQAKALSVELEINFETRGKRGQ |
| | | DAMYEYMAQACAGNRVRRSLCEGTL |
| | | LLWCDIIGQTTYRDLKL |
| Granzyme B | 51 | QPILLLLAFLLLPRADAGEIIGGHE |
| | | AKPHSRPYMAYLMIWDQKSLKRCGG |
| | | FLIQDDFVLTAAHCWGSSINVTLGA |
| | | HNIKEQEPTQQFIPVKRPIPHPAYN |
| | | PKNFSNDIMLLQLERKAKRTRAVQP |
| | | LRLPSNKAQVKPGQTCSVAGWGQTA |
| | | PLGKHSHTLQEVKMTVQEDRKCESD |
| | | LRHYYDSTIELCVGDPEIKKTSFKG |
| | | DSGGPLVCNKVAQGIVSYGRNNGMP |
| | | PRACTKVSSFVHWIKKTMKRH |
| Bax | 52 | DGSGEQPRGGGPTSSEQIMKTGALL |
| | | LQGFIQDRAGRMGGEAPELALDPVP |
| | | QDASTKKLSECLKRIGDELDSNMEL |
| | | QRMIAAVDTDSPREVFFRVAADMFS |
| | | DGNFNWGRVVALFYFASKLVLKALC |
| | | TKVPELIRTIMGWTLDFLRERLLGW |
| | | IQDQGGWDGLLSYFGTPTWQTVTIF |
| | | VAGVLTASLTIWKKMG |
| XIAP | 53 | SRGSEFMTFNSFEGSKTCVPADINK |
| | | EEEFVEEFNRLKTFANFPSGSPVSA |
| | | STLARAGFLYTGEGDTVRCFSCHAA |
| | | VDRWQYGDSAVGRHRKVSPNCRFIN |
| | | GFYLENSATQSTNSGIQNGQYKVEN |
| | | YLGSRDHFALDRPSETHADYLLRTG |
| | | QVVDISDTIYPRNPAMYSEEARLKS |
| | | FQNWPDYAHLTPRELASAGLYYTGI |
| | | GDQVQCFCCGGKLKNWEPCDRAWSE |
| | | HRRHFPNCFFVLGRNLNIRSESDAV |
| | | SSDRNFPNSTNLPRNPSMADYEARI |
| | | FTFGTWIYSVNKEQLARAGFYALGE |
| | | GDKVKCFHCGGGLTDWKPSEDPWEQ |
| | | HAKWYPGCKYLLEQKGQEYINNIHL |
| | | THSLEECLVRTTEKTPSLTRRIDD |
| | | TIFQNPMVQEAIRMGFSFKDIKKIM |
| | | EEKIQISGSNYKSLEVLVADLVNAQ |
| | | KDSMQDESSQTSLQKEISTEEQLRR |
| | | LQEEKLCKICMDRNIAIVFVPCGHL |
| | | VTCKQCAEAVDKCPMCYTVITFKQK |
| | | IFMS |
| ZF | 54 | GGCGTAGCCGATGTCGCG |
| binding | | |
| site | | |
| 4X ZF | 55 | cgggtttcgtaacaatcgcatgagg |
| binding site | | attcgcaacgccttcGGCGTAGCCG |
| | | ATGTCGCGctcccgtctcagtaaag |
| | | gtcGGCGTAGCCGATGTCGCGcaat |
| | | cggactgccttcgtacGGCGTAGCC |
| | | GATGTCGCGcgtatcagtcgcctcg |
| | | gaacGGCGTAGCCGATGTCGCG |
| Exemplary | 56 | TCTAGAGGGTATATAATGGGGGCCA |
| core promoter | | |
| sequence | | |
| Exemplary | 57 | cgggtttcgtaacaatcgcatgagg |
| ITM- | | attcgcaacgccttcGGCGTAGCCG |
| responsive | | ATGTCGCGctcccgtctcagtaaag |
| promoter | | gtcGGCGTAGCCGATGTCGCGcaat |
| | | cggactgccttcgtacGGCGTAGCC |
| | | GATGTCGCGcgtatcagtcgcctcg |
| | | gaacGGCGTAGCCGATGTCGCGcat |
| | | tcgtaagaggctcactctcccttac |
| | | acggagtggataACTAGTTCTAGAG |
| | | GGTATATAATGGGGGCCA |
| Exemplary | 58 | TCTGTTCCTGTTAATCAACCTCTGG |
| WPRE | | ATTACAAAATTTGTGAAAGATTGAC |
| | | TGATATTCTTAACTATGTTGCTCCT |
| | | TTTACGCTGTGTGGATATGCTGCTT |
| | | TAATGCCTCTGTATCATGCTATTGC |
| | | TTCCCGTACGGCTTTCGTTTTCTCC |
| | | TCCTTGTATAAATCCTGGTTGCTGT |
| | | CTCTTTATGAGGAGTTGTGGCCCGT |
| | | TGTCCGTCAACGTGGCGTGGTGTGC |
| | | TCTGTGTTTGCTGACGCAACCCCCA |
| | | CTGGCTGGGGCATTGCCACCACCTG |
| | | TCAACTCCTTTCTGGGACTTTCGCT |
| | | TTCCCCCTCCCGATCGCCACGGCAG |
| | | AACTCATCGCCGCCTGCCTTGCCCG |
| | | CTGCTGGACAGGGGCTAGGTTGCTG |
| | | GGCACTGATAATTCCGTGGTGTTGT |
| | | CGGGGAAGCTGACGTCCTTTCCATG |
| | | GCTGCTCGCCTGTGTTGCCAACTGG |
| | | ATCCTGCGCGGGACGTCCTTCTGCT |
| | | ACGTCCCTTCGGCTCTCAATCCAGC |
| | | GGACCTCCCTTCCCGAGGCCTTCTG |
| | | CCGGTTCTGCGGCCTCTCCCGCGTC |
| | | TTCGCTTTCGGCCTCCGACGAGTCG |
| | | GATCTCCCTTTGGGCCGCCTCCCCG |
| | | CCTGTTTCGCCTCGGCGTCCGGTCC |
| | | GTGTTGCTTGGTCGTCACCTGTGCA |
| | | GAATTGCGAACCATGGATTCCA |
| Exemplary | 59 | TCTGTTCCTGTTAATCAACCTCTGG |
| WPRE | | ATTACAAAATTTGTGAAAGATTGAC |
| | | TGATATTCTTAACTATGTTGCTCCT |
| | | TTTACGCTGTGTGGATATGCTGCTT |
| | | TAATGCCTCTGTATCATGCTATTGC |
| | | TTCCCGTACGGCTTTCGTTTTCTCC |
| | | TCCTTGTATAAATCCTGGTTGCTGT |
| | | CTCTTTATGAGGAGTTGTGGCCCGT |
| | | TGTCCGTCAACGTGGCGTGGTGTGC |
| | | TCTGTGTTTGCTGACGCAACCCCCA |
| | | CTGGCTGGGGCATTGCCACCACCTG |
| | | TCAACTCCTTTCTGGGACTTTCGCT |
| | | TTCCCCCTCCCGATCGCCACGGCAG |
| | | AACTCATCGCCGCCTGCCTTGCCCG |
| | | CTGCTGGACAGGGGCTAGGTTGCTG |
| | | GGCACTGATAATTCCGTGGTGTTGT |
| | | CGGGGAAATCATCGTCCTTTCCTTG |
| | | GCTGCTCGCCTGTGTTGCCAACTGG |
| | | ATCCTGCGCGGGACGTCCTTCTGCT |
| | | ACGTCCCTTCGGCTCTCAATCCAGC |
| | | GGACCTCCCTTCCCGAGGCCTTCTG |
| | | CCGGTTCTGCGGCCTCTCCCGCGTC |
| | | TTCGCTTTCGGCCTCCGACGAGTCG |
| | | GATCTCCCTTTGGGCCGCCTCCCCG |
| | | CCTGTTTCGCCTCGGCGTCCGGTCC |
| | | GTGTTGCTTGGTCGTCACCTGTGCA |
| | | GAATTGCGAACCATGGATTCCA |

### Example 5: IMiD Regulated Expression of IL-12 Payload in an IMiD-on System

Efficiency of IMiD regulated expression of IL-12 payload was assessed in a system where addition of an IMiD increases expression of IL-12 through degradation of a degron-linked repressor.

### Materials and Methods

50,000 U87MG cells were seeded in 24 w dish 18-14 hours before transduction then transduced with 25 k pg of degron-linked repressor (SB03759, SB03936, or SB04397) and 25 k pg of IL12 reporter construct (SB04640) (virus was quantified by p24 ELISA). A construct encoding IL-12 linked to an ITM-responsive promoter including an EFS promoter sequence with a zinc finger binding domain composed of four ZF10-1 binding sites upstream of the EFS promoter sequence (SEQ ID NO: 67) was used as a reporter.

For Pomalidomide titration experiments, two days post transduction, cells were split into drug free media or 1 uM pomalidomide conditions. Then 3 days later, 100 k cells of each condition were seeded into a 24 w plate and 24 hours later, supernatant was collected and assayed using IL12 p70 ELISA kit (#D1200 R&D Systems) to quantity IL-12.

For kinetic experiment studies, transduced cells were seeded 100 k cells per well in 24 w plate. Then 24 hours later, supernatant was replaced with drug-free or 1 uM Pomalidomide or Iberdomide. Supernatant was harvested at indicated elapsed times (3 hours, 6 hours, 12 hours, 16 hours and 24 hours) post drug treatment.

The constructs assessed are shown in Table 11. Each construct format is described from 5′ to 3′ (ORF from N to C terminus) with sequences described in Table 11. Sequences for constructs are shown in Table 12.

TABLE 11
Constructs tested for IMiD-ON regulated
expression of IL-12 payload

| Construct | |
|---|---|
| Name | Description |
| SB04397 | all E. coli cod opt: d913-A(EAAAK)3A-ZF10-1- |
| | minKrab-BFP-2x(AuSLDE) |
| SB03936 | d913-A(EAAAK)-HDAC4-ZF10 |
| SB03759 | d913-A(EAAAK)3A-ZF10-1-minKrab-AuSLDE |
| SB04640 | 4xZF10-1 binding site_pEF1alpha: IL12 |

### Results

Regulated IL-12 expression in response to immunomodulatory drug (IMiD) pomalidomide was assessed for constructs that include d913 degrons in various orientations and using various linkers for drug-regulatable systems where addition of an IMiD leads to degradation of degron-linked repressors resulting in increased expression of a payload (“IMiD ON”).

Degron-linked repressors were constructed to repress expression of the payload IL-12 in drug-free conditions through an inducible transcription modulator (ITM) featuring a transcriptional repressor and a DNA binding domain that in the absence of drug binds to the promoter operably linked to IL-12. The ITM is linked to a degron such that transcription repression is released when the degron-linked ITM is degraded upon addition of the IMiD Pomalidomide.

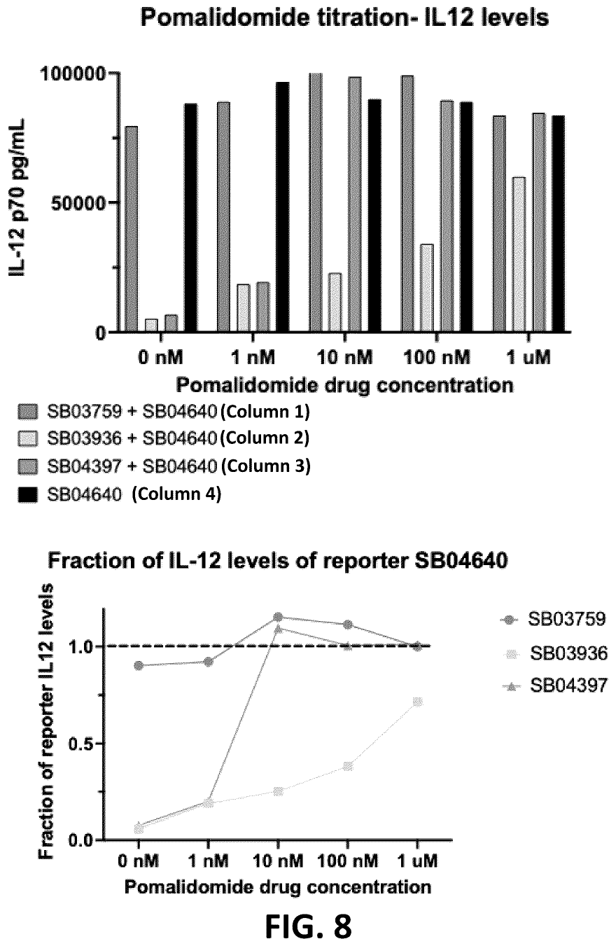

Three different degron-linked repressors were assessed for their ability to regulate IL-12 expression under drug-free conditions or increasing concentrations of Pomalidomide (1 nM, 10 nM, 100 nM, and 1 uM). As shown in FIG. 8, IL-12 expression was increased upon addition of the IMiD for constructs SB03936 and SB04397. IL-12 levels are shown in the top panel. Shown in the bottom panel is the quantification of IL-12 restoration expression to non-repressed levels when the degron-linked repressor is degraded with the addition of increasing concentrations of pomalidomide. IL-12 levels are displayed as a fraction of IL12 expression levels quantified in cells transduced with constitutive IL-12 reporter. IL-12 expression recovers to reporter only levels in cells regulated by SB04397 degron-linked repressor and treated with 10 nM or higher concentrations of Pomalidomide. The results demonstrate IMiD-dependent regulation of IL-12 production where addition of the IMiDs increased expression of IL-12 in an “IMiD-ON” system featuring degron-linked repressors.

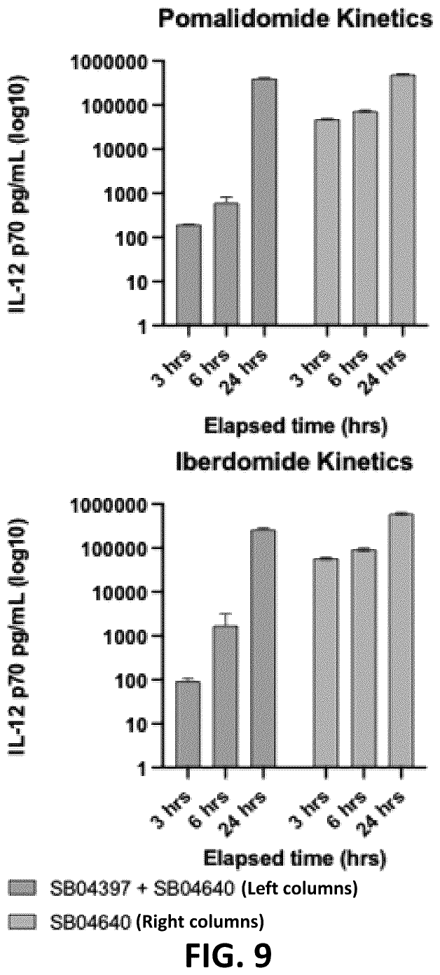

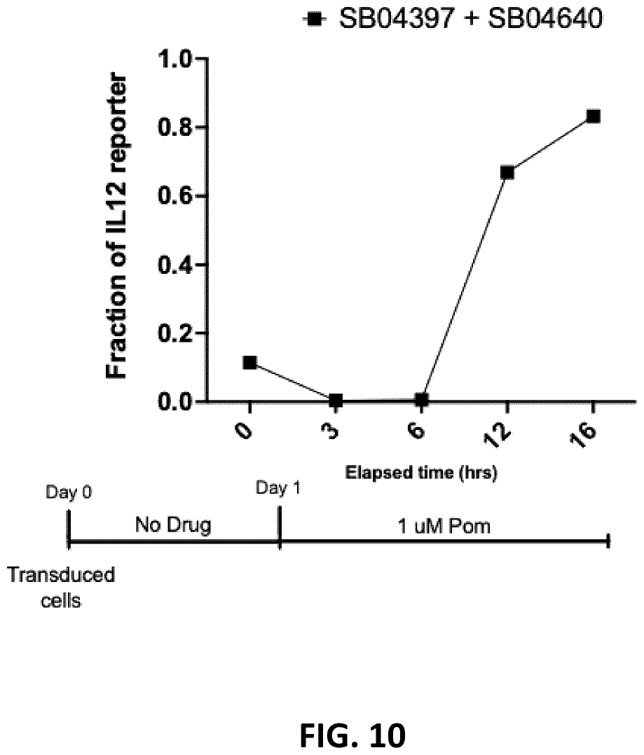

Kinetics were then assessed for the degron-linked repressor SB04397. The IMiDs Pomalidomide and Iberdomide were also compared. As shown in FIG. 9, IL-12 expression levels increased in supernatant gathered from cells over time for both 1 uM pomalidomide (top panel) and 1 uM Iberdomide (bottom panel) treatments for cells transfected with both the IL-12 reporter SB04640 and the degron-linked repressor SB04397 (left columns) compared to the reporter only control (right columns). Kinetics were further assessed at additional time points for the degron-linked repressor SB04397 and the IMiD Pomalidomide. As shown in FIG. 10, IL-12 expression levels (as assessed normalized to IL-12 expression levels in cells transduced with the SB04640 reporter only) demonstrated increased IL-12 expression at hours 12 and 16 upon addition of 1 uM Pomalidomide. These results demonstrate the rapid activation of the IMiD ON switch, with expression of IL-12 detected within 24 hours of adding IMiDs and initiating the degradation of the degron-linked repressor.

TABLE 12
Sequences

| | SEQ | |
|---|---|---|
| | ID | |
| Name | NO | Sequence |
| SB03759 | 61 | FNVLMVHKRSHTGERPLQCEICGFT |
| (AA) | | CRQKGNLLRHIKLHTGEKPFKCHLC |
| | | NYACQRRDALAEAAAKEAAAKEAAA |
| | | KADYKDHDGDYKDHDIDYKDDDDKM |
| | | APKKKRKVTCRSRPGERPFQCRICM |
| | | RNFSRRHGLDRHTRTHTGEKPFQCR |
| | | ICMRNFSDHSSLKRHLRTHTGSQKP |
| | | FQCRICMRNFSVRHNLTRHLRTHTG |
| | | EKPFQCRICMRNFSDHSNLSRHLKT |
| | | HTGSQKPFQCRICMRNFSQRSSLVR |
| | | HLRTHTGEKPFQCRICMRNFSESGH |
| | | LKRHLRTHLRGSGGGGSGGTRTLVT |
| | | FKDVFVDFTREEWKLLDTAQQIVYR |
| | | NVMLENYKNLVSLGY |
| SB03759 | 62 | TTCAACGTGCTGATGGTGCACAAGC |
| (Nucleic | | GGAGCCACACCGGCGAAAGACCTCT |
| Acid) | | GCAGTGTGAAATCTGCGGCTTCACC |
| | | TGTCGGCAGAAGGGCAACCTGCTGC |
| | | GGCACATCAAACTGCACACAGGCGA |
| | | GAAGCCCTTCAAGTGCCACCTGTGC |
| | | AATTACGCCTGCCAGAGAAGAGATG |
| | | CCCTGGCCGAAGCCGCCGCTAAAGA |
| | | AGCTGCTGCCAAAGAGGCCGCTGCC |
| | | AAGGCCGATTACAAGGACCACGATG |
| | | GCGACTATAAGGATCACGACATCGA |
| | | CTACAAGGACGATGACGACAAGATG |
| | | GCCCCTAAGAAAAAGCGGAAAGTGA |
| | | CCTGCAGAAGCAGACCCGGCGAGAG |
| | | GCCTTTCCAGTGCAGAATCTGCATG |
| | | CGGAACTTCAGCAGACGGCACGGCC |
| | | TGGACAGACACACCAGAACACATAC |
| | | CGGGGAGAAGCCATTTCAGTGCCGG |
| | | ATCTGTATGCGCAATTTCTCCGACC |
| | | ACAGCAGCCTGAAGCGGCACCTGAG |
| | | AACACACACAGGCAGCCAGAAACCA |
| | | TTCCAATGTCGGATCTGCATGAGAA |
| | | ATTTCAGCGTGCGGCACAACCTGAC |
| | | CAGACACCTGAGGACCCATACTGGC |
| | | GAAAAACCCTTCCAGTGTCGCATAT |
| | | GTATGAGGAACTTTAGCGACCACTC |
| | | CAACCTGAGCCGCCACCTGAAAACC |
| | | CACACCGGATCTCAGAAACCTTTTC |
| | | AGTGTAGGATATGCATGCGCAACTT |
| | | TAGCCAGCGGAGCAGCCTTGTGCGC |
| | | CATCTGAGAACTCACACTGGGGAGA |
| | | AACCCTTTCAATGCCGAATATGCAT |
| | | GCGAAATTTTTCCGAGAGCGGCCAC |
| | | CTCAAGCGGCATCTGCGTACACACC |
| | | TTAGAGGATCTGGCGGAGGTGGCAG |
| | | CGGAGGCACAAGAACCCTGGTCACC |
| | | TTCAAGGACGTGTTCGTGGACTTCA |
| | | CCCGGGAAGAGTGGAAGCTGCTGGA |
| | | TACAGCCCAGCAGATCGTGTACCGG |
| | | AACGTGATGCTGGAAAACTACAAGA |
| | | ATCTGGTGTCCCTGGGCTACTGACT |
| | | GCAGGCATGCGTGACTGACTGAGGC |
| | | CGCGACTCTAGTTTAAACATTTATT |
| | | TATTTATTTATTTAacatcggttcc |
| | | CTGTTTAATATTTAAACAGtgcggt |
| | | aagcATTTATTTATTTATTTATTTA |
| | | acatcggttccCTGTTTAATATTTA |
| | | AACAGa |
| SB03936 | 63 | FNVLMVHKRSHTGERPLQCEICGFT |
| (AA) | | CRQKGNLLRHIKLHTGEKPFKCHLC |
| | | NYACQRRDALAEAAAKEAAAKEAAA |
| | | KASSQSHPDGLSGRDQPVELLNPAR |
| | | VNHMPSTVDVATALPLQVAPSAVPM |
| | | DLRLDHQFSLPVAEPALREQQLQQE |
| | | LLALKQKQQIQRQILIAEFQRQHEQ |
| | | LSRQHEAQLHEHIKQQQEMLAMKHQ |
| | | QELLEHQRKLERHRQEQELEKQHRE |
| | | QKLQQLKNKEKGKESAVASTEVKMK |
| | | LQEFVLNKKKALAHRNLNHCISSDP |
| | | RYWYGKTQHSSLDQSSPPQSGVSTS |
| | | YNHPVLGMYDAKDDFPLRKTASEPN |
| | | LKLRSRLKQKVAERRSSPLLRRKDG |
| | | PVVTALKKRPLDVTDSACSSAPGSG |
| | | PSSPNNSSGSVSAENGIAPAVPSIP |
| | | AETSLAHRLVAREGSAAPLPLYTSP |
| | | SLPNITLGLPATGPSAGTAGQQDTE |
| | | RLTLPALQQRLSLFPGTHLTPYLST |
| | | SPLERDGGAAHSPLLQHMVLLEQPP |
| | | AQAPLVTGLGALPLHAQSLVGADRV |
| | | SPSIHKLRQHRPLGRTQSAPLPQNA |
| | | QALQHLVIQQQHQQFLEKHKQQFQQ |
| | | QQLQMNKIIPKPSEPARQPESHPEE |
| | | TEEELREHQALLDEPYLDRLPGQKE |
| | | AHAQAGVQVKQEPIESDEEEAEPPR |
| | | EVEPGQRQPSEQELLFRQQALLLEQ |
| | | QRIHQLRNYQASMEAAGIPVSFGGH |
| | | RPLSRAQSSPASATFPVSVQEPPTK |
| | | PRFTTGLVYDTLMLKHQCTCGSSSS |
| | | HPEHAGRIQSIWSRLQETGLRGKCE |
| | | CIRGRKATLEELQTVHSEAHTLLYG |
| | | TNPLNRQKLDSKKLLGSLASVFVRL |
| | | PCGGVGVDSDTIWNEVHSAGAARLA |
| | | VGCVVELVFKVATGELKNGFAVVRP |
| | | PGHHAEESTPMGFCYFNSVAVAAKL |
| | | LQQRLSVSKILIVDWDVHHGNGTQQ |
| | | AFYSDPSVLYMSLHRYDDGNFFPGS |
| | | GAPDEVGTGPGVGFNVNMAFTGGLD |
| | | PPMGDAEYLAAFRTVVMPIASEFAP |
| | | DVVLVSSGFDAVEGHPTPLGGYNLS |
| | | ARCFGYLTKQLMGLAGGRIVLALEG |
| | | GHDLTAICDASEACVSALLGNELDP |
| | | LPEKVLQQRPNANAVRSMEKVMEIH |
| | | SKYWRCLQRTTSTAGRSLIEAQTCE |
| | | NEEAETVTAMASLSVGVKPAEKRPD |
| | | EEPMEEEPPLGGGGSGGTDYKDHDG |
| | | DYKDHDIDYKDDDDKMAPKKKRKVT |
| | | CRSRPGERPFQCRICMRNFSRRHGL |
| | | DRHTRTHTGEKPFQCRICMRNFSDH |
| | | SSLKRHLRTHTGSQKPFQCRICMRN |
| | | FSVRHNLTRHLRTHTGEKPFQCRIC |
| | | MRNFSDHSNLSRHLKTHTGSQKPFQ |
| | | CRICMRNFSQRSSLVRHLRTHTGEK |
| | | PFQCRICMRNFSESGHLKRHLRTHL |
| | | RGS |
| SB03936 | 64 | TTCAACGTGCTGATGGTGCACAAGC |
| (Nucleic | | GGAGCCACACCGGCGAAAGACCTCT |
| Acid) | | GCAGTGTGAAATCTGCGGCTTCACC |
| | | TGTCGGCAGAAGGGCAACCTGCTGC |
| | | GGCACATCAAACTGCACACAGGCGA |
| | | GAAGCCCTTCAAGTGCCACCTGTGC |
| | | AATTACGCCTGCCAGAGAAGAGATG |
| | | CCCTGGCCGAAGCCGCCGCTAAAGA |
| | | AGCTGCTGCCAAAGAGGCCGCTGCC |
| | | AAGGCCagctcccaaagccatccag |
| | | atggactttctggccgagaccagcc |
| | | agtggagctgctgaatcctgcccgc |
| | | gtgaaccacatgcccagcacggtgg |
| | | atgtggccacggcgctgcctctgca |
| | | agtggccccctcggcagtgcccatg |
| | | gacctgcgcctggaccaccagttct |
| | | cactgcctgtggcagagccggccct |
| | | gcgggagcagcagctgcagcaggag |
| | | ctcctggcgctcaagcagaagcagc |
| | | agatccagaggcagatcctcatcgc |
| | | tgagttccagaggcagcacgagcag |
| | | ctctcccggcagcacgaggcgcagc |
| | | tccacgagcacatcaagcaacaaca |
| | | ggagatgctggccatgaagcaccag |
| | | caggagctgctggaacaccagcgga |
| | | agctggagaggcaccgccaggagca |
| | | ggagctggagaagcagcaccgggag |
| | | cagaagctgcagcagctcaagaaca |
| | | aggagaagggcaaagagagtgccgt |
| | | ggccagcacagaagtgaagatgaag |
| | | ttacaagaatttgtcctcaataaaa |
| | | agaaggcgctggcccaccggaatct |
| | | gaaccactgcatttccagcgaccct |
| | | cgctactggtacgggaaaacgcagc |
| | | acagttcccttgaccagagttctcc |
| | | accccagagcggagtgtcgacctcc |
| | | tataaccacccggtcctgggaatgt |
| | | acgacgccaaagatgacttccctct |
| | | taggaaaacagcttctgaaccgaat |
| | | ctgaaattacggtccaggctaaagc |
| | | agaaagtggccgaaagacggagcag |
| | | ccccctgttacgcaggaaagacggg |
| | | ccagtggtcactgctctaaaaaagc |
| | | gtccgttggatgtcacagactccgc |
| | | gtgcagcagcgccccaggctccgga |
| | | cccagctcacccaacaacagctccg |
| | | ggagcgtcagcgcggagaacggtat |
| | | cgcgcccgccgtccccagcatcccg |
| | | gcggagacgagtttggcgcacagac |
| | | ttgtggcacgagaaggctcggccgc |
| | | tccacttcccctctacacatcgcca |
| | | tccttgcccaacatcacgctgggcc |
| | | tgcctgccaccggcccctctgcggg |
| | | cacggcgggccagcaggacaccgag |
| | | agactcacccttcccgccctccagc |
| | | agaggctctcccttttccccggcac |
| | | ccacctcactccctacctgagcacc |
| | | tcgcccttggagcgggacggagggg |
| | | cagcgcacagccctcttctgcagca |
| | | catggtcttactggagcagccaccg |
| | | gcacaagcacccctcgtcacaggcc |
| | | tgggagcactgcccctccacgcaca |
| | | gtccttggttggtgcagaccgggtg |
| | | tccccctccatccacaagctgcggc |
| | | agcaccgcccactggggcggaccca |
| | | gtcggccccgctgccccagaacgcc |
| | | caggctctgcagcacctggtcatcc |
| | | agcagcagcatcagcagtttctgga |
| | | gaaacacaagcagcagttccagcag |
| | | cagcaactgcagatgaacaagatca |
| | | tccccaagccaagcgagccagcccg |
| | | gcagccggagagccacccggaggag |
| | | acggaggaggagctccgtgagcacc |
| | | aggctctgctggacgagccctacct |
| | | ggaccggctgccggggcagaaggag |
| | | gcgcacgcacaggccggcgtgcagg |
| | | tgaagcaggagcccattgagagcga |
| | | tgaggaagaggcagagcccccacgg |
| | | gaggtggagccgggccagcgccagc |
| | | ccagtgagcaggagctgctcttcag |
| | | acagcaagccctcctgctggagcag |
| | | cagcggatccaccagctgaggaact |
| | | accaggcgtccatggaggccgccgg |
| | | catccccgtgtccttcggcggccac |
| | | aggcctctgtcccgggcgcagtcct |
| | | cacccgcgtctgccaccttccccgt |
| | | gtctgtgcaggagccccccaccaag |
| | | ccgaggttcacgacaggcctcgtgt |
| | | atgacacgctgatgctgaagcacca |
| | | gtgcacctgcgggagtagcagcagc |
| | | caccccgagcacgccgggaggatcc |
| | | agagcatctggtcccgcctgcagga |
| | | gacgggcctccggggcaaatgcgag |
| | | tgcatccgcggacgcaaggccaccc |
| | | tggaggagctacagacggtgcactc |
| | | ggaagcccacaccctcctgtatggc |
| | | acgaaccccctcaaccggcagaaac |
| | | tggacagtaagaaacttctaggctc |
| | | gctcgcctccgtgttcgtccggctc |
| | | ccttgcggtggtgttggggggacag |
| | | tgacaccatatggaacgaggtgcac |
| | | tcggcgggggcagcccgcctggctg |
| | | tgggctgcgtggtagagctggtctt |
| | | caaggtggccacaggggagctgaag |
| | | aatggctttgctgtggtccgccccc |
| | | ctggacaccatgcggaggagagcac |
| | | gcccatgggcttttgctacttcaac |
| | | tccgtggccgtggcagccaagcttc |
| | | tgcagcagaggttgagcgtgagcaa |
| | | gatcctcatcgtggactgggacgtg |
| | | caccatggaaacgggacccagcagg |
| | | ctttctacagcgaccctagcgtcct |
| | | gtacatgtccctccaccgctacgac |
| | | gatgggaacttcttcccaggcagcg |
| | | gggctcctgatgaggtgggcacagg |
| | | gcccggcgtgggtttcaacgtcaac |
| | | atggctttcaccggcggcctggacc |
| | | cccccatgggagacgctgagtactt |
| | | ggcggccttcagaacggtggtcatg |
| | | ccgatcgccagcgagtttgccccgg |
| | | atgtggtgctggtgtcatcaggctt |
| | | cgatgccgtggagggccaccccacc |
| | | cctcttgggggctacaacctctccg |
| | | ccagatgcttcgggtacctgacgaa |
| | | gcagctgatgggcctggctggcggc |
| | | cggattgtcctggccctcgagggag |
| | | gccacgacctgaccgccatttgcga |
| | | cgcctcggaagcatgtgtttctgcc |
| | | ttgctgggaaacgagcttgatcctc |
| | | tcccagaaaaggttttacagcaaag |
| | | acccaatgcaaacgctgtccgttcc |
| | | atggagaaagtcatggagatccaca |
| | | gcaagtactggcgctgcctgcagcg |
| | | cacaacctccacagcggggcgttct |
| | | ctgatcgaggctcagacttgcgaga |
| | | acgaagaagccgagacggtcaccgc |
| | | catggcctcgctgtccgtgggcgtg |
| | | aagcccgccgaaaagagaccagatg |
| | | aggagcccatggaagaggagccgcc |
| | | cctgGGCGGCGGAGGATCTGGCGGC |
| | | ACAGATTACAAAGACCACGACGGCG |
| | | ACTACAAGGATCACGACATCGATTA |
| | | CAAGGACGACGATGACAAGATGGCC |
| | | CCTAAGAAAAAGCGGAAAGTGACCT |
| | | GCAGAAGCAGACCCGGCGAAAGACC |
| | | CTTCCAGTGCCGGATCTGCATGCGG |
| | | AACTTCAGCAGAAGGCACGGCCTGG |
| | | ACAGACACACCAGAACACACACAGG |
| | | CGAGAAGCCTTTCCAGTGTAGAATC |
| | | TGTATGCGCAATTTCAGCGACCACA |
| | | GCAGCCTGAAGCGGCACCTGAGAAC |
| | | ACATACCGGCAGCCAGAAACCATTT |
| | | CAATGCCGCATCTGTATGAGAAACT |
| | | TCTCCGTGCGGCACAACCTGACCAG |
| | | ACACCTGAGGACCCACACCGGGGAG |
| | | AAACCCTTCCAATGCAGAATATGCA |
| | | TGAGGAATTTCTCCGACCACTCCAA |
| | | CCTGAGCCGCCACCTGAAAACCCAT |
| | | ACAGGCTCTCAAAAGCCCTTTCAAT |
| | | GTCGGATATGTATGCGGAATTTTTC |
| | | CCAGCGGAGCAGCCTCGTGCGCCAT |
| | | CTGAGAACTCACACTGGGGAAAAGC |
| | | CATTTCAGTGCCGTATATGCATGCG |
| | | CAATTTCTCTGAGAGCGGCCACCTG |
| | | AAGAGACATCTGCGGACACACCTGA |
| | | GAGGCTCTTGA |
| SB04397 | 65 | FNVLMVHKRSHTGERPLQCEICGFT |
| (AA) | | CRQKGNLLRHIKLHTGEKPFKCHLC |
| | | NYACQRRDALAEAAAKEAAAKEAAA |
| | | KADYKDHDGDYKDHDIDYKDDDDKM |
| | | APKKKRKVTCRSRPGERPFQCRICM |
| | | RNFSRRHGLDRHTRTHTGEKPFQCR |
| | | ICMRNFSDHSSLKRHLRTHTGSQKP |
| | | FQCRICMRNFSVRHNLTRHLRTHTG |
| | | EKPFQCRICMRNFSDHSNLSRHLKT |
| | | HTGSQKPFQCRICMRNFSQRSSLVR |
| | | HLRTHTGEKPFQCRICMRNFSESGH |
| | | LKRHLRTHLRGSGGGGSGGTRTLVT |
| | | FKDVFVDFTREEWKLLDTAQQIVYR |
| | | NVMLENYKNLVSLGYGGGGSGGTMS |
| | | ELIKENMHMKLYMEGTVDNHHFKCT |
| | | SEGEGKPYEGTQTMRIKVVEGGPLP |
| | | FAFDILATSFLYGSKTFINHTQGIP |
| | | DFFKQSFPEGFTWERVTTYEDGGVL |
| | | TATQDTSLQDGCLIYNVKIRGVNFT |
| | | SNGPVMQKKTLGWEAFTETLYPADG |
| | | GLEGRNDMALKLVGGSHLIANIKTT |
| | | YRSKKPAKNLKMPGVYYVDYRLERI |
| | | KEANNETYVEQHEEVAVARYCDLPS |
| | | KLGHKLN |
| SB04397 | 66 | TTCAACGTATTGATGGTTCATAAAC |
| (Nucleic | | GTTCACACACAGGGGAACGTCCCCT |
| Acid) | | TCAGTGCGAAATCTGCGGGTTTACC |
| | | TGCCGCCAAAAAGGCAATTTATTGC |
| | | GTCACATTAAGCTTCATACTGGCGA |
| | | AAAACCCTTCAAGTGTCATCTGTGT |
| | | AACTATGCGTGCCAGCGTCGCGACG |
| | | CATTAGCTGAAGCCGCCGCCAAAGA |
| | | AGCTGCCGCCAAAGAGGCGGCCGCG |
| | | AAGGCGGACTACAAAGATCATGACG |
| | | GTGATTATAAAGATCACGATATTGA |
| | | CTATAAGGACGACGACGACAAAATG |
| | | GCACCAAAGAAGAAACGCAAAGTCA |
| | | CATGCCGCTCGCGCCCTGGGGAGCG |
| | | TCCCTTTCAGTGTCGTATTTGCATG |
| | | CGCAATTTTTCCCGCCGCCATGGAC |
| | | TGGATCGTCATACACGCACACATAC |
| | | AGGAGAAAAGCCATTCCAGTGTCGT |
| | | ATCTGTATGCGCAATTTCTCAGACC |
| | | ATTCCAGTCTTAAGCGTCATCTGCG |
| | | TACCCACACGGGATCTCAAAAACCG |
| | | TTCCAGTGCCGCATCTGTATGCGTA |
| | | ACTTTTCGGTACGCCATAACTTAAC |
| | | GCGTCATTTACGCACCCATACAGGG |
| | | GAGAAGCCGTTTCAGTGCCGCATTT |
| | | GCATGCGTAACTTCTCCGATCATAG |
| | | CAATCTGAGCCGCCACTTGAAAACA |
| | | CATACGGGTTCGCAGAAGCCGTTTC |
| | | AGTGTCGCATTTGTATGCGCAATTT |
| | | CTCCCAACGTTCCTCTTTGGTACGC |
| | | CACTTGCGTACACACACGGGGGAGA |
| | | AACCATTCCAATGTCGCATCTGTAT |
| | | GCGCAACTTTTCTGAGTCAGGGCAT |
| | | CTGAAGCGTCACCTGCGTACTCACC |
| | | TGCGCGGTTCGGGTGGAGGCGGCAG |
| | | TGGCGGTACGAGAACCTTGGTCACT |
| | | TTCAAAGATGTTTTCGTAGACTTTA |
| | | CACGCGAGGAGTGGAAGCTGCTGGA |
| | | TACCGCACAGCAAATCGTGTATCGT |
| | | AACGTCATGCTTGAGAACTATAAGA |
| | | ACCTTGTCAGCTTGGGGTACGGTGG |
| | | AGGCGGCAGTGGCGGTACGATGAGC |
| | | GAGCTTATTAAGGAGAACATGCATA |
| | | TGAAACTGTACATGGAGGGCACCGT |
| | | TGACAACCATCACTTTAAGTGCACA |
| | | TCAGAAGGCGAAGGTAAACCATATG |
| | | AGGGCACACAAACCATGCGCATCAA |
| | | GGTCGTGGAAGGAGGGCCTCTGCCT |
| | | TTCGCCTTCGACATTTTAGCCACAA |
| | | GCTTTCTGTACGGCAGCAAAACTTT |
| | | CATCAATCATACTCAGGGGATCCCT |
| | | GATTTCTTTAAACAGTCATTCCCCG |
| | | AGGGTTTCACTTGGGAGCGTGTCAC |
| | | TACCTACGAAGATGGTGGAGTGTTG |
| | | ACAGCGACTCAGGACACTAGTCTGC |
| | | AGGATGGGTGTTTGATCTACAATGT |
| | | CAAAATCCGTGGGGTTAACTTTACG |
| | | TCCAATGGCCCCGTGATGCAGAAAA |
| | | AGACGCTGGGGTGGGAGGCATTTAC |
| | | TGAGACGCTTTACCCGGCCGATGGA |
| | | GGACTTGAAGGACGTAACGATATGG |
| | | CACTGAAGTTAGTGGGTGGATCTCA |
| | | CCTTATTGCCAATATCAAGACAACG |
| | | TATCGTTCTAAAAAGCCAGCAAAGA |
| | | ACTTAAAGATGCCAGGGGTGTATTA |
| | | TGTGGACTACCGTTTAGAGCGTATT |
| | | AAGGAAGCGAATAACGAGACGTATG |
| | | TGGAACAACATGAGGTGGCCGTGGC |
| | | CCGTTACTGTGACCTGCCGTCGAAA |
| | | TTAGGGCACAAGCTTAATTGACTGC |
| | | AGGCATGCGTGACTGACTGAGGCCG |
| | | CGACTCTAGTTTAAACATTTATTTA |
| | | TTTATTTATTTAacatcggttccCT |
| | | GTTTAATATTTAAACAGtgcggtaa |
| | | gcATTTATTTATTTATTTATTTAac |
| | | atcggttccCTGTTTAATATTTAAA |
| | | CAGa |
| EFS | 67 | cgggtttcgtaacaatcgcatgagg |
| promoter | | attcgcaacgccttcGGCGTAGCCG |
| (bold) + 4 | | ATGTCGCGctcccgtctcagtaaag |
| upstream | | gtcGGCGTAGCCGATGTCGCGcaat |
| ZF10-1 | | cggactgccttcgtacGGCGTAGCC |
| binding | | GATGTCGCGcgtatcagtcgcctcg |
| sites | | gaacGGCGTAGCCGATGTCGCGcat |
| | | tcgtaagaggctcactctcccttac |
| | | acggagtggataACTAGTGGATCTG |
| | | CGATCGCTCCGGTGCCCGTCAGTGG |
| | | GCAGAGCGCACATCGCCCACAGTCC |
| | | CCGAGAAGTTGGGGGGAGGGGTCGG |
| | | CAATTGAACCGGTGCCTAGAGAAGG |
| | | TGGCGCGGGGTAAACTGGGAAAGTG |
| | | ATGTCGTGTACTGGCTCCGCCTTTT |
| | | TCCCGAGGGTGGGGGAGAACCGTAT |
| | | ATAAGTGCAGTAGTCGCCGTGAACG |
| | | TTCTTTTTCGCAACGGGTTTGCCGC |
| | | CAGAACACAGCTGAAGCTTCGAGGG |
| | | GCTCGCATCTCTCCTTCACGCGCCC |
| | | GCCGCCCTACCTGAGGCCGCCATCC |
| | | ACGCCGGTTGAGTCGCGTTCTGCCG |
| | | CCTCCCGCCTGTGGTGCCTCCTGAA |
| | | CTGCGTCCGCCGTCTAGGTAAGTTT |
| | | AAAGCTCAGGTCGAGACCGGGCCTT |
| | | TGTCCGGCGCTCCCTTGGAGCCTAC |
| | | CTAGACTCAGCCGGCTCTCCACGCT |
| | | TTGCCTGACCCTGCTTGCTCAACTC |
| | | TACGTCTTTGTTTCGTTTTCTGTTC |
| | | TGCGCCGTTACAGATCCAAGCTGTG |
| | | ACCGGCGCCTAC |

While the present disclosure has been particularly shown and described with reference to a preferred embodiment and various alternate embodiments, it will be understood by persons skilled in the relevant art that various changes in form and details can be made therein without departing from the spirit and scope of the present disclosure and appended claims.

All references, issued patents and patent applications cited within the body of the instant specification are hereby incorporated by reference in their entirety, for all purposes.

1. A chimeric polypeptide comprising:
(a) an inducible transcription modulator (ITM), wherein the ITM comprises a transcriptional repressor domain and a DNA binding domain; and
(b) a degron,
wherein the degron is operably linked to the ITM, and
wherein the transcriptional repressor domain is selected from the group consisting of: a KRAB repression domain, an HDAC4 domain, a SCX HLH domain, a ID1 HLH domain, a HERC2 Cyt-b5 domain, a TWST1 HLH domain, an NKX22 homeodomain, an ID3 HLH domain, and a TWST2 HLH domain.

2. The chimeric polypeptide of claim 1, wherein the transcriptional repressor domain comprises the KRAB repression domain
optionally wherein the KRAB repression domain comprises minKRAB,
optionally wherein the KRAB repression domain comprises the amino acid sequence of SEQ ID NO: 2,
optionally wherein the KRAB repression domain comprises a KRAB repressor domain variant of SEQ ID NO: 2 and comprises one or more amino acid substitutions selected from the group consisting of: W27L, K28L, D31A, T32A, Q34A, Q35A, R39E, L43S, T57C, K58C, P59C, V61Y, I62Y, I62A, L63W, L63Y, L63E, R64F, R64W, R64E, L65F, L65E, L65W, E66V, K67F, G68F, and E69F,
optionally wherein the KRAB repression domain comprises a KRAB repressor domain variant of SEQ ID NO: 2 and comprises one or more amino acid substitutions selected from: Q34A/Q35A, I62A, T57C/K58C/P59C, D31A/T32A, L63W/R64W/L65W, E66V, L63E/R64E/L65E, R64F/L65F, W27L/K28L KRAB, R39E, K67F/G68F/E69F, and V61Y/I62Y/L63Y,
optionally wherein the KRAB repression domain comprises the amino acid sequence of SEQ ID NO: 3.

3. The chimeric polypeptide of claim 1, wherein the transcriptional repressor domain comprises the HDAC4 repression domain,
optionally wherein the HDAC4 repression domain comprises the amino acid sequence of SEQ ID NO: 4.

4. The chimeric polypeptide of claim 1,
wherein the DNA binding domain comprises a zinc finger (ZF) protein domain, optionally wherein the ZF protein domain is modular in design and is composed of a zinc finger array (ZFA) of zinc finger motifs,
optionally wherein the ZF protein domain comprises one to ten zinc finger motifs,
optionally wherein the ZF protein domain comprises six zinc finger motifs,
optionally wherein the ZF protein domain comprises SEQ ID NO: 5.

5. The chimeric polypeptide of claim 1,
wherein:
a. the transcriptional repressor domain is N-terminal to the DNA binding domain or C-terminal to the DNA binding domain; and/or
b. the transcriptional repressor domain and the DNA binding domain are separated by a first peptide linker,
optionally wherein the first peptide linker comprises an amino acid sequence selected from the group consisting of: GGGGSGGT (SEQ ID NO: 60); and/or
c. the ITM is a synthetic transcription modulator.

6. The chimeric polypeptide of claim 1 wherein:
(a) the degron is selected from the group consisting of: HCV NS4 degron, PEST (two copies of residues 277-307 of human IκBα), GRR (residues 352-408 of human p105), DRR (residues 210-295 of yeast Cdc34), SNS (tandem repeat of SP2 and NB (SP2-NB-SP2 of influenza A or influenza B), RPB (four copies of residues 1688-1702 of yeast RPB), SPmix (tandem repeat of SP1 and SP2 (SP2-SP1-SP2-SP1-SP2 of influenza A virus M2 protein), NS2 (three copies of residues 79-93 of influenza A virus NS protein), ODC (residues 106-142 of ornithine decarboxylase), Nek2A, mouse ODC (residues 422-461), mouse ODC_DA (residues 422-461 of mODC including D433A and D434A point mutations), an APC/C degron, a COP1 E3 ligase binding degron motif, a CRL4-Cdt2 binding PIP degron, an actinfilin-binding degron, a KEAP1 binding degron, a KLHL2 and KLHL3 binding degron, an MDM2 binding motif, an N-degron, a hydroxyproline modification in hypoxia signaling, a phytohormone-dependent SCF-LRR-binding degron, an SCF ubiquitin ligase binding phosphodegron, a phytohormone-dependent SCF-LRR-binding degron, a DSGxxS phospho-dependent degron (SEQ ID NO: 68), an Siah binding motif, an SPOP SBC docking motif, and a PCNA binding PIP box; and/or
(b) the degron comprises a cereblon (CRBN) polypeptide substrate domain capable of binding CRBN in response to an immunomodulatory drug (IMiD),
optionally wherein the CRBN polypeptide substrate domain is selected from the group consisting of: IKZF1, IKZF3, CKla, ZFP91, GSPT1, MEIS2, GSS E4F1, ZN276, ZN517, ZN582, ZN653, ZN654, ZN692, ZN787, and ZN827, or a fragment thereof that is capable of drug-inducible binding of CRBN,
optionally, wherein the CRBN polypeptide substrate domain comprises a chimeric fusion product of native CRBN polypeptide sequences,
optionally wherein the CRBN polypeptide substrate domain comprises a IKZF3/ZFP91/IKZF3 chimeric fusion product having the amino acid sequence of FNVLMVHKRSHTGERPLQCEICGF TCRQKGNLLRHIKLHTGEKPFKCHLCNYAC QRRDAL (SEQ ID NO: 6); and/or
(c) the IMiD is an FDA-approved drug,
optionally wherein the IMiD is selected from the group consisting of: thalidomide, lenalidomide, and pomalidomide,
optionally wherein the ITM is N-terminal to the degron or C-terminal to the degron, optionally wherein the ITM is separated from the degron by a second peptide linker, optionally wherein the second peptide linker comprises an amino acid sequence selected from the group consisting of: GSGSGSGS (SEQ ID NO: 7), KEGS (SEQ ID NO: 8), EGK, EAAAK (SEQ ID NO: 9), and AAPAKQE (SEQ ID NO: 10) or the second peptide linker comprises an amino acid sequence selected from the group consisting of:
(SEQ ID NO: 12)
AAPAKQEAAAPAKQEAAAPAKQ
EAAAPAPAAKAEAPAAAPAAKA
and
(SEQ ID NO: 13)
AEAAAKEAAAKEAAAKA

7. An expression cassette comprising a promoter operably linked to a polynucleotide sequence encoding the chimeric polypeptide of claim 1,
optionally wherein the promoter comprises a constitutive promoter selected from the group consisting of: CMV, EFS, SFFV, SV40, MND, PGK, UbC, hEF1a, hCAGG, hACTb, heIF4A1, hGAPDH, hGRP78, hGRP94, hHSP70, hKINb, and hUBIb or the promoter comprises an inducible promoter, optionally wherein the inducible promoter comprises a minimal promoter and a response element selected from the group consisting of: NFkB response element, CREB response element, NFAT response element, SRF response element 1, SRF response element 2, AP1 response element, TCF-LEF response element promoter fusion, Hypoxia responsive element, SMAD binding element, STAT3 binding site, inducer molecule responsive promoters, and tandem repeats thereof,
optionally wherein the promoter comprises a synthetic promoter,
optionally wherein the polynucleotide sequence encoding the chimeric polypeptide further encodes a 3′untranslated region (UTR) comprising an mRNA-destabilizing element,
optionally wherein the mRNA-destabilizing element is selected from the group consisting of: an AU-rich element and a stem-loop destabilizing element.

8. An expression system comprising the expression cassette of claim 7, and a target expression cassette comprising an ITM-responsive promoter operably linked to a gene of interest,
optionally wherein the ITM-responsive promoter comprises a promoter sequence and a sequence that binds to the DNA binding domain of the ITM,
optionally wherein the sequence that binds to the DNA binding domain comprises one or more zinc finger binding sites,
optionally wherein the sequence that binds to the DNA binding domain comprises one or more zinc finger binding sites having the amino acid sequence of SEQ ID NO: 54, optionally wherein the sequence that binds to the DNA binding domain comprises four of more zinc finger binding sites,
optionally wherein the sequence that binds to the DNA binding domain comprises the amino acid sequence of SEQ ID NO: 55,
optionally wherein the promoter sequence of the ITM-responsive promoter comprises a constitutive promoter sequence selected from the group consisting of: CMV, EFS, SFFV, SV40, MND, PGK, UbC, EF1a, hCAGG, hACTb, heIF4A1, hGAPDH,
hGRP78, hGRP94, hHSP70, hKINb, and hUBIb,
optionally wherein the promoter sequence of the ITM-responsive promoter comprises a minimal promoter,
optionally wherein the ITM-responsive promoter comprises a synthetic promoter, optionally wherein the gene of interest encodes a therapeutic polypeptide selected from the group consisting of: a cytokine, a chemokine, a homing molecule, a growth factor, a cell death regulator, a co-activation molecule, a tumor microenvironment modifier a, a receptor, a ligand, an antibody, a polynucleotide, a peptide, and an enzyme,
optionally wherein the expression system comprises a heterologous construct comprising both of: (i) the expression cassette of claim 7 and (ii) the target expression cassette,
optionally wherein the expression system comprises a first heterologous construct comprising the expression cassette of claim 7 and a second heterologous construct comprising the target expression cassette.

9. An isolated cell comprising the expression cassette of claim 7.

10. The isolated cell of claim 9, wherein:
1 the cell is a human cell; and/or
2 the cell is a stem cell; and/or
3 the cell is an immune cell; and/or
4 the cell is selected from the group consisting of: a T cell, a CD8+ T cell, a CD4+ T cell, a gamma-delta T cell, a cytotoxic T lymphocyte (CTL), a regulatory T cell, a viral-specific T cell, a Natural Killer T (NKT) cell, a Natural Killer (NK) cell, a B cell, a tumor-infiltrating lymphocyte (TIL), an innate lymphoid cell, a mast cell, an eosinophil, a basophil, a neutrophil, a myeloid cell, a macrophage, a monocyte, a dendritic cell, an erythrocyte, a platelet cell, a human embryonic stem cell (ESC), an ESC-derived cell, a pluripotent stem cell, a mesenchymal stromal cell (MSC), an induced pluripotent stem cell (iPSC), and an iPSC-derived cell.

11. A genetic switch for inhibiting repression of a gene of interest, comprising: the chimeric polypeptide of claim 1 and a ligand, wherein binding of the ligand to the degron induces degradation of the chimeric polypeptide, thereby inhibiting repression of the gene of interest, wherein the gene of interest is operably linked to an ITM-responsive promoter,
optionally wherein the ligand comprises an immunomodulatory drug (IMiD) that promotes ubiquitin pathway-mediated degradation of the chimeric polypeptide,
optionally wherein the IMiD is an FDA-approved drug,
optionally wherein the IMiD is selected from the group consisting of: thalidomide, lenalidomide, and pomalidomide.

12. A method of inhibiting repression of a gene of interest, comprising:
a. providing a cell comprising an expression system comprising (i) an expression cassette encoding the chimeric polypeptide of claim 1, and (ii) a target expression cassette comprising an ITM-responsive promoter operably linked to a gene of interest; and
b. inducing degradation of the chimeric polypeptide by contacting the transformed cell with a ligand that promotes degradation of the chimeric polypeptide.

13. The method of claim 12, wherein
the method further comprises culturing the cell under conditions suitable for expression of the chimeric polypeptide.

14. A method of producing a cell that is capable of drug-regulated transcriptional repression, the method comprising transforming the cell with the expression cassette claim 7.
